(19) United States
(12) Patent Application Publication
(10) Pub. No.: US 2009/0054623 A1
(43) Pub. Date: Feb. 26, 2009

(54) Lipo-Conjugation of Peptides

(75) Inventor: Shawn DeFrees, North Wales, PA (US)

Correspondence Address: MORGAN, LEWIS & BOCKIUS LLP (SF), One Market, Spear Street Tower, Suite 2800, San Francisco, CA, 94105, US

(73) Assignee: Neose Technologies, Inc.

(21) Appl. No.: 11/792,610

(22) PCT Filed: Dec. 19, 2005
(86) PCT No.: PCT/US2005/046198

§ 371 Date: Apr. 21, 2008

Related U.S. Application Data
(60) Provisional application No. 60/637,179, filed on Dec. 17, 2004.

(51) Int. Cl. C07K 2/00

(52) U.S. Cl. 530/345

(57) ABSTRACT

The present invention provides peptide conjugates that are formed between a modified lipid and a glycosyl residue and/or an amino acid residue on a peptide. The modified lipid includes a modifying group and a lipid linking group. Exemplary lipid linking groups include myristoyl, palmitoyl, and isoprenyl moieties.

### CROSS-REFERENCES TO RELATED APPLICATIONS

The present application is a U.S. national phase application of PCT Application No. PCT/US2005/046198, filed Dec. 19, 2005, which claims priority to U.S. Provisional Patent Application No. 60/637,179, filed on Dec. 17, 2004, each of which is incorporated herein by reference in their entirety for all purposes.

### BACKGROUND OF THE INVENTION

The administration of modified peptides for improving the pharmacokinetics of peptides and engendering a particular physiological response is well known in the medicinal arts. Unfortunately, a principal factor limiting the use of modified therapeutic peptides is the difficulty inherent in engineering an expression system to express a peptide having a precisely defined and controlled modification pattern.

Improperly or incompletely modified peptides can be toxic, immunogenic, or may provide only suboptimal potency and rapid clearance rates. Indeed, one of the most important problems in the production of modified peptide therapeutics is the loss of peptide activity that is directly attributable to the non-selective nature of the chemistries utilized to conjugate a water-soluble polymer.

Polyethylene glycol is an exemplary water soluble polymer that is well known in the art and frequently employed as a peptide conjugate. The principal mode of attachment of PEG, and its derivatives, to peptides is a non-specific bonding through a peptide amino acid residue. For example, U.S. Pat. No. 4,088,538 discloses an enzymatically active polymer-enzyme conjugate of an enzyme covalently bound to PEG. Similarly, U.S. Pat. No. 4,496,689 discloses a covalently attached complex of α-1 proteinase inhibitor with a polymer such as PEG or methoxypoly(ethyleneglycol) (“(m-) PEG”). Abuchowski et al. (J. Biol. Chem. 252: 3578 (1977)) discloses the covalent attachment of (m-) PEG to an amine group of bovine serum albumin. U.S. Pat. No. 4,414,147 discloses a method of rendering interferon less hydrophobic by conjugating it to an anhydride of a dicarboxylic acid, such as poly(ethylene succinic anhydride). PCT WO 87/00056 discloses conjugation of PEG and poly(oxyethylated) polyols to such proteins as interferon-β, interleukin-2 and immunotoxins. EP 154,316 discloses and claims chemically modified lymphokines, such as IL-2 containing PEG bonded directly to at least one primary amino group of the lymphokine. U.S. Pat. No. 4,055,635 discloses pharmaceutical compositions of a water-soluble complex of a proteolytic enzyme linked covalently to a polymeric substance such as a polysaccharide.

In each of the methods described above, poly(ethyleneglycol) is added in a random, non-specific manner to reactive residues on a peptide backbone. However, for the production of therapeutic peptides, it is clearly preferable to utilize a derivatization strategy that is highly predictable and which results in the formation of a specifically labeled, readily characterizable, essentially homogeneous product. A promising alternative route to preparing specifically labeled peptides is through the use of enzymes.

Post-expression in vitro enzymatic modification of peptides is an attractive strategy for the preparation of modified proteins. Enzyme-based syntheses have the advantages of regioselectivity and stereoselectivity such that proteins with custom designed modification patterns can be produced. Additional benefits of enzymatic syntheses include the ability to perform syntheses using unprotected substrates. The use of unprotected substrates would require fewer steps for in vitro modification of peptides than do the currently practiced random addition methods, and also would reduce the toxicity of the production process.

An example of successful enzymatic post-expression in vitro modification of peptides has been achieved for the in vitro glyco-modification of glycotherapeutics, e.g., glycopeptides. A comprehensive toolbox of recombinant eukaryotic glycosyltransferases has become available, making in vitro enzymatic synthesis of mammalian glycoconjugates with custom designed glycosylation patterns and glycosyl structures possible. See, for example, U.S. Pat. Nos. 5,876,980; 6,030,815; 5,728,554; 5,922,577; and WO/9831826; US2003180835; and WO 03/031464.

However, glycoproteins are not the only therapeutic proteins for which modified derivatives could be useful for therapeutic purposes. Indeed, many lipid containing and membrane proteins are also important in disease processes, and thus, their modified derivatives are likely to prove useful as therapeutics.

Thus, what is clearly needed in the art is an industrially relevant method that utilizes enzymatic conjugation to specifically conjugate a modified lipid to a peptide, thereby providing a method for controlling and manipulating the specific position of modification of certain therapeutic lipopeptides.

The present invention answers this need. The invention provides modified therapeutic peptides in which a modified lipid moiety is conjugated onto the peptides. The invention thus provides a route to new therapeutic conjugates and addresses the need for more stable and therapeutically effective therapeutic species.

### SUMMARY OF THE INVENTION

Incorrect modification of therapeutic peptides can produce a peptide that is inactive, antigenic and/or has unfavorable pharmacokinetics. Accordingly, considerable efforts are expended to develop recombinant expression systems capable of producing therapeutic proteins that are modified in a biologically appropriate manner, such that they retain the correct, and/or possibly enhanced, biological activity. Until now, this approach has been hampered by numerous shortcomings, including cost, and heterogeneity of the resulting products.

Bacterial expression of peptide therapeutics combined with post-expression in vitro enzymatic modification of therapeutic peptides offers a number of advantages compared to traditional chemical modification methods. Advantages of enzymatic modification methods include reduced potential exposure to adventitious agents, increased homogeneity of product, and cost reduction.

The present invention provides peptide conjugates which include a peptide and a modified lipid. In these peptide conjugates, the modified lipid includes at least one lipid linking group and at least one modifying group, and the modifying group is covalently attached to the peptide at a preselected glycosyl and/or amino acid residue of said peptide via a lipid linking group.

In some embodiments of the invention, the modified lipid is conjugated to the peptide through a sulfur, nitrogen, or oxygen atom on the peptide. In an exemplary embodiment, the atom is a sulfur or a nitrogen atom. In an exemplary embodiment, the lipid linking group can be

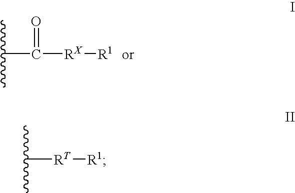

in which R^{X} is a member selected from substituted or unsubstituted, saturated or unsaturated C₁-C₄₀ alkyl and R¹ is a member selected from a water soluble polymer, a water insoluble polymer, a therapeutic moiety, and a diagnostic moiety. R^{T} includes at least one moiety which has a structure according to the formula:

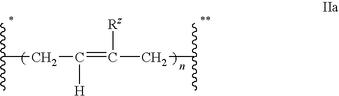

in which R^{z} is a member selected from H and substituted or unsubstituted methyl, and [symbol] and [symbol] describe the points of attachment between said moiety and the remainder of the main chain of the modified lipid. The index n is an integer from 1 to 20.

In one aspect the invention exploits the natural recognition mechanisms of lipid transferase enzymes. In another aspect, invention exploits the recognition that certain classes of enzymes, which are typically degradative, can be made to run in a synthetic, rather than a degradative mode. Exemplary enzymes are those that are involved in the cleavage of bonds that include an acyl-containing component, such as an ester or an amide. Thus, enzymes of use in the present invention include, but are not limited to, proteases, lipases, acylases, acyltransferases, and esterases.

The invention also provides methods of improving pharmacological parameters of peptide therapeutics. For example, the invention provides a means for altering the pharmacokinetics, pharmacodynamics and bioavailability of peptide therapeutics, e.g., cytokines, antibodies, growth hormones, enzymes, and lipoproteins. In particular, the invention provides a method for lengthening the in vivo half-life of a peptide therapeutic by conjugating a water-soluble polymer to the therapeutic moiety through a lipid linking group. In an exemplary embodiment, covalent attachment of polymers, such as polyethylene glycol (PEG), e.g, m-PEG, to a therapeutic moiety affords conjugates having in vivo residence times, and pharmacokinetic and pharmacodynamic properties that are enhanced relative to the unconjugated therapeutic.

As discussed in the preceding section, art-recognized methods of covalent PEGylation rely on chemical conjugation through reactive groups, typically amines, on amino acids or carbohydrates. A major shortcoming of chemical conjugation of PEG to proteins or lipoproteins is lack of selectivity, which often results in attachment of PEG at sites implicated in protein bioactivity.

In contrast to art-recognized chemical conjugation methods, the present invention provides a novel, enzymatically-mediated strategy for highly selective conjugation, e.g., PEGylation, directed to one or more specific locations on an amino acid residue of a peptide. In an exemplary embodiment of the invention, site directed attachment of PEG is provided by in vitro enzymatic acylation of specific residues comprising an activated PEG substituted lipid compound.

In an exemplary embodiment, the present invention provides a peptide conjugate in which the modified lipid has a structure which is a member selected from the formulas:

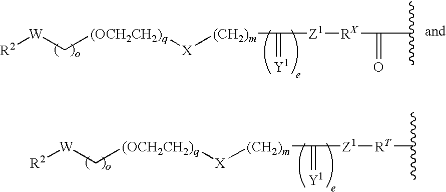

in which R² is a member selected from H, substituted or unsubstituted alkyl, substituted or unsubstituted aryl, substituted or unsubstituted heteroaryl, substituted or unsubstituted heterocycloalkyl, substituted or unsubstituted heteroalkyl, e.g., acetal, OHC—, H₂N—CH₂CH₂—, HS—CH₂CH₂—, and —(CH₂)_{q}C(Y¹)Z¹; -sugar-nucleotide, or protein. R^{T} includes at least one moiety which has a structure according to the formula:

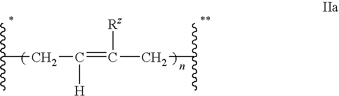

in which R^{z} is a member selected from H and substituted or unsubstituted methyl, and [symbol] and [symbol] describe the points of attachment between said moiety and the remainder of the main chain of the modified lipid. The index n is an integer selected from 1 to 20. The index q is an integer selected from 1 to 2500. R^{X} is a member selected from substituted or unsubstituted, saturated or unsaturated C₁-C₄₀ alkyl. The index e is an integer selected from 0 and 1. The index m and o are integers independently selected from 0 to 20. Z¹ is a member selected from a bond, O, S, N—R⁴, —(CH₂)ₚC(Y²)V, —(CH₂)ₚU(CH₂)ₛC(Y²)ᵥ. X, Y¹, Y², W and U are independently selected from O, S, N—R⁴. V is a member selected from OH, NH₂, halogen, S—R⁵, the alcohol component of activated esters, the amine component of activated amides, sugar-nucleotides, and proteins. The indices p, s and v are integers independently selected from 0 to 20. R³, R⁴ and R⁵ are independently selected from H, substituted or unsubstituted alkyl, substituted or unsubstituted heteroalkyl, substituted or unsubstituted aryl, substituted or unsubstituted heterocycloalkyl and substituted or unsubstituted heteroaryl.

In another exemplary embodiment, the present invention provides a peptide conjugate in which the modified lipid has a structure which is a member selected from the formulas:

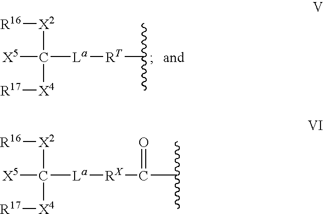

in which L^{a} is a linker selected from a bond, substituted or unsubstituted alkyl and substituted or unsubstituted heteroalkyl. R^{T} includes at least one moiety which has a structure according to the formula:

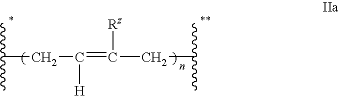

in which R^{z} is a member selected from H and substituted or unsubstituted methyl, and [symbol] and [symbol] describe the points of attachment between said moiety and the remainder of the main chain of the modified lipid. The index n is an integer selected from 1 to 20. R^{X} is a member selected from substituted or unsubstituted, saturated or unsaturated C₁-C₄₀ alkyl. The indices R¹⁶ and R¹⁷ are independently selected polymeric arms. The indices X² and X⁴ are independently selected linkage fragments joining polymeric moieties R¹⁶ and R¹⁷ to C. X⁵ is a non-reactive group.

In another exemplary embodiment, the present invention provides a peptide conjugate in which the modified lipid has a structure which is a member selected from the formulas:

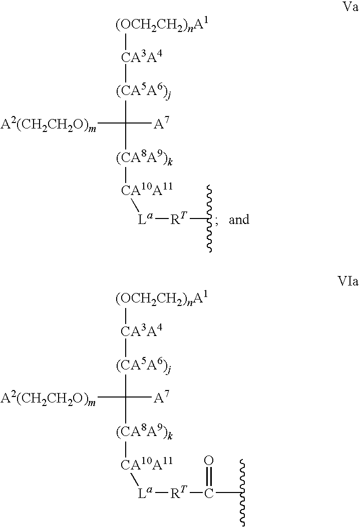

in which A¹, A², A³, A⁴, A⁵, A⁶, A⁷, A⁸, A⁹, A¹⁰ and A¹¹ are members independently selected from H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —NA¹²A¹³, —OA¹² and —SiA¹²A¹³. A¹² and A¹³ are members independently selected from substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

The invention also provides methods of making the peptide conjugates, as well as pharmaceutical formulations which include a peptide conjugate along with a pharmaceutically acceptable excipient.

Additional aspects, advantages and objects of the present invention will be apparent from the detailed description that follows.

### BRIEF DESCRIPTION OF THE DRAWINGS

FIG. 1 is a table of the peptides to which one or more lipid linking groups can be attached to order to provide the peptide conjugates of the invention.

FIG. 2 is a table of palmitoylation consensus sequences.

### DETAILED DESCRIPTION OF THE INVENTION

### I. Abbreviations

PEG, poly(ethyleneglycol); m-PEG, methoxy-poly(ethylene glycol); PPG, poly(propyleneglycol); m-PPG, methoxy-poly(propylene glycol); Fuc, fucosyl; Gal, galactosyl; GalNAc, N-acetylgalactosaminyl; Glc, glucosyl; GlcNAc, N-acetylglucosaminyl; Man, mannosyl; ManAc, mannosaminyl acetate; Sia, sialic acid; and NeuAc, N-acetylneuraminyl.

### II. Definitions

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization are those well known and commonly employed in the art. Standard techniques are used for nucleic acid and peptide synthesis. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document. The nomenclature used herein and the laboratory procedures in analytical chemistry, and organic synthetic described below are those well known and commonly employed in the art. Standard techniques, or modifications thereof, are used for chemical syntheses and chemical analyses.

“Peptide” refers to a polymer in which the monomers are amino acids and are joined together through amide bonds, alternatively referred to as a polypeptide. Additionally, unnatural amino acids, for example, β-alanine, phenylglycine and homoarginine are also included. Amino acids that are not gene-encoded may also be used in the present invention. Furthermore, amino acids that have been modified to include reactive groups, glycosylation sites, polymers, therapeutic moieties, biomolecules and the like may also be used in the invention. All of the amino acids used in the present invention may be either the D- or L-isomer. The L-isomer is generally preferred. In addition, other peptidomimetics are also useful in the present invention. As used herein, “peptide” refers to both glycosylated and unglycosylated peptides. Also included are peptides that are incompletely glycosylated by a system that expresses the peptide. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983).

The term “peptide conjugate,” refers to species of the invention in which a peptide is conjugated with a modified lipid as set forth herein.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M)

(see, e.g., Creighton, Proteins (1984)).

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “mutating” or “mutation,” as used in the context of altering the structure or enzymatic activity of a wild-type enzyme, refers to the deletion, insertion, or substitution of any nucleotide or amino acid residue, by chemical, enzymatic, or any other means, in a polynucleotide sequence encoding a that enzyme or the amino acid sequence of a wild-type enzyme, respectively, such that the amino acid sequence of the resulting enzyme is altered at one or more amino acid residues. The site for such an activity-altering mutation may be located anywhere in the enzyme, but is preferably within the active site of the enzyme.

The term “lipid”, as used here, refers to naturally occurring and synthetic hydrophobic species that include an isoprene moiety, a fatty acid moiety (carboxylic acid covalently attached to a substituted or unsubstituted C₂ to C₆₀ alkyl moiety), and combinations thereof. Examples of lipids include e.g., farnesyl moieties, geranylgeranyl moieties, lauric acid (CH₃(CH₂)₁₀COOH, n-dodecanoic acid), myristic acid (CH₃(CH₂)₁₂COOH, n-tetradecanoic acid), palmitic acid (CH₃(CH₂)₁₄COOH, n-hexadecanoic acid), stearic acid, (CH₃(CH₂)₁₆COOH, n-octadecanoic acid), arachidic acid (CH₃(CH₂)₁₈COOH, n-eicosanoic acid), lignoceric acid (CH₃(CH₂)₂₂COOH, n-tetracosanoic acid), palmitoleic acid (CH₃(CH₂)₅CH═CH(CH₂)₇COOH, cis-9-hexadecenoic acid), oleic acid (CH₃(CH₂)₇CH═CH(CH₂)₇COOH, cis-9-octadecenoic acid), linoleic acid, α-linoleic acid, arachidonic acid, triacylaglycerols, phospholipids, glycerophospholipids, glycolipids such as galactolipids, glycerophospholipids (also known as phosphoglycerides), sphingolipids such as ceramide, sphingomyelin, dolichol, glucocerebrosides, globosides and gangliosides.

As used herein, the term “modified lipid,” refers to a naturally- or non-naturally-occurring lipid that is enzymatically added onto an amino acid or a glycosyl residue of a peptide in a process of the invention. The “modified lipid” is covalently functionalized with a “modifying group.” Useful modifying groups include, but are not limited to, PEG moieties, therapeutic moieties, diagnostic moieties, biomolecules and the like. The modifying group is preferably not a naturally occurring, or an unmodified lipid. The locus of functionalization with the modifying group is selected such that it does not prevent the “modified lipid” from being added enzymatically to a peptide.

The term “water-soluble” refers to moieties that have some detectable degree of solubility in water. Methods to detect and/or quantify water solubility are well known in the art. Exemplary water-soluble polymers include peptides, saccharides, poly(ethers), poly(amines), poly(carboxylic acids) and the like. Peptides can have mixed sequences of be composed of a single amino acid, e.g., poly(lysine). An exemplary polysaccharide is poly(sialic acid). An exemplary poly(ether) is poly(ethylene glycol). Poly(ethylene imine) is an exemplary polyamine, and poly(acrylic) acid is a representative poly(carboxylic acid).

The polymer backbone of the water-soluble polymer can be poly(ethylene glycol) (i.e. PEG). However, it should be understood that other related polymers are also suitable for use in the practice of this invention and that the use of the term PEG or poly(ethylene glycol) is intended to be inclusive and not exclusive in this respect. The term PEG includes poly(ethylene glycol) in any of its forms, including alkoxy PEG, difunctional PEG, multiarmed PEG, forked PEG, branched PEG, pendent PEG (i.e. PEG or related polymers having one or more functional groups pendent to the polymer backbone), or PEG with degradable linkages therein.

The polymer backbone can be linear or branched. Branched polymer backbones are generally known in the art. Typically, a branched polymer has a central branch core moiety and a plurality of linear polymer chains linked to the central branch core. PEG is commonly used in branched forms that can be prepared by addition of ethylene oxide to various polyols, such as glycerol, pentaerythritol and sorbitol. The central branch moiety can also be derived from several amino acids, such as lysine. The branched poly(ethylene glycol) can be represented in general form as R(—PEG-OH)ₘ in which R represents the core moiety, such as glycerol or pentaerythritol, and m represents the number of arms. Multi-armed PEG molecules, such as those described in U.S. Pat. No. 5,932,462, which is incorporated by reference herein in its entirety, can also be used as the polymer backbone.

Many other polymers are also suitable for the invention. Polymer backbones that are non-peptidic and water-soluble, with from 2 to about 300 termini, are particularly useful in the invention. Examples of suitable polymers include, but are not limited to, other poly(alkylene glycols), such as poly(propylene glycol) (“PPG”), copolymers of ethylene glycol and propylene glycol and the like, poly(oxyethylated polyol), poly(olefinic alcohol), poly(vinylpyrrolidone), poly(hydroxypropylmethacrylamide), poly(α-hydroxy acid), poly(vinyl alcohol), polyphosphazene, polyoxazoline, poly(N-acryloylmorpholine), such as described in U.S. Pat. No. 5,629,384, which is incorporated by reference herein in its entirety, and copolymers, terpolymers, and mixtures thereof. Although the molecular weight of each chain of the polymer backbone can vary, it is typically in the range of from about 100 Da to about 100,000 Da, often from about 6,000 Da to about 80,000 Da.

The “area under the curve” or “AUC”, as used herein in the context of administering a peptide drug to a patient, is defined as total area under the curve that describes the concentration of drug in systemic circulation in the patient as a function of time from zero to infinity.

The term “half-life” or “t½”, as used herein in the context of administering a peptide drug to a patient, is defined as the time required for plasma concentration of a drug in a patient to be reduced by one half. There may be more than one half-life associated with the peptide drug depending on multiple clearance mechanisms, redistribution, and other mechanisms well known in the art. Usually, alpha and beta half-lives are defined such that the alpha phase is associated with redistribution, and the beta phase is associated with clearance. However, with protein drugs that are, for the most part, confined to the bloodstream, there can be at least two clearance half-lives. Further explanation of “half-life” is found in Pharmaceutical Biotechnology (1997, DFA Crommelin and R D Sindelar, eds., Harwood Publishers, Amsterdam, pp 101-120).

The term “glycoconjugation,” as used herein, refers to the enzymatically mediated conjugation of a modified sugar species to an amino acid or glycosyl residue of a polypeptide. A subgenus of “glycoconjugation” is “glycoPEGylation,” in which the modifying group of the modified sugar is poly(ethylene glycol), or an alkyl (e.g., m-PEG) or reactive (e.g., H₂N-PEG, HOOC-PEG) derivative thereof.

The term “lipoconjugation,” as used herein, refers to the enzymatically mediated conjugation of a modified lipid to an amino acid or glycosyl residue of a polypeptide. A subgenus of “lipoconjugation” is “lipoPEGylation,” in which the modifying group of the modified lipid is poly(ethylene glycol), or an alkyl (e.g., m-PEG) or reactive (e.g., H₂N-PEG, HOOC-PEG) derivative thereof.

The terms “large-scale” and “industrial-scale” are used interchangeably and refer to a reaction cycle that produces at least about 250 mg, preferably at least about 500 mg, and more preferably at least about 1 gram of lipoconjugate at the completion of a single reaction cycle.

The term, “lipid linking group,” as used herein refers to a lipid residue to which a modifying group (e.g., PEG moiety, therapeutic moiety, biomolecule) is covalently attached; the glycosyl linking group joins the modifying group to the remainder of the conjugate. In the methods of the invention, the “lipid linking group” becomes covalently attached to a glycosylated or unglycosylated peptide, thereby linking the modifying group to an amino acid and/or glycosyl residue on the peptide. A “lipid linking group” is generally derived from a “modified lipid” by the enzymatic attachment of the “modified lipid” to an amino acid and/or glycosyl residue of the peptide.

The term, “lipid transfer enzyme,” as used herein refers to an enzyme that is capable of covalently attaching a lipid residue to an amino acid residue or a glycosyl residue. Examples of lipid transfer enzymes useful in the practice of the invention include but are not limited to, wild-type and mutant proteases, lipases, esterases, acylases and acyltransferases. In some exemplary embodiments, the enzymes may be wild-type or mutant prenyltransferases (e.g., farnesyltransferases, and geranylgeranyl transferases); N-myristoyltransferases or palmitoyltransferases.

The term “targeting moiety,” as used herein, refers to species that will selectively localize in a particular tissue or region of the body. The localization is mediated by specific recognition of molecular determinants, molecular size of the targeting agent or conjugate, ionic interactions, hydrophobic interactions and the like. Other mechanisms of targeting an agent to a particular tissue or region are known to those of skill in the art. Exemplary targeting moieties include antibodies, antibody fragments, transferrin, HS-glycoprotein, coagulation factors, serum proteins, β-glycoprotein, G-CSF, GM-CSF, M-CSF, EPO and the like.

As used herein, “therapeutic moiety” means any agent useful for therapy including, but not limited to, antibiotics, anti-inflammatory agents, anti-tumor drugs, cytotoxins, and radioactive agents. “Therapeutic moiety” includes prodrugs of bioactive agents, constructs in which more than one therapeutic moiety is bound to a carrier, e.g, multivalent agents. Therapeutic moiety also includes proteins and constructs that include proteins. Exemplary proteins include, but are not limited to, Granulocyte Colony Stimulating Factor (GCSF), Granulocyte Macrophage Colony Stimulating Factor (GMCSF), Interferon (e.g., Interferon-α, -β, -γ), Interleukin (e.g., Interleukin II), serum proteins (e.g., Factors VII, VIIa, VIII, IX, and X), Human Chorionic Gonadotropin (HCG), Follicle Stimulating Hormone (FSH) and Lutenizing Hormone (LH) and antibody fusion proteins (e.g. Tumor Necrosis Factor Receptor ((TNFR)/Fc domain fusion protein)).

As used herein, “pharmaceutically acceptable carrier” includes any material, which when combined with the conjugate retains the conjugates' activity and is non-reactive with the subject's immune systems. Examples include, but are not limited to, any of the standard pharmaceutical carriers such as a phosphate buffered saline solution, water, emulsions such as oil/water emulsion, and various types of wetting agents. Other carriers may also include sterile solutions, tablets including coated tablets and capsules. Typically such carriers contain excipients such as starch, milk, sugar, certain types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well known conventional methods.

As used herein, “administering,” means oral administration, administration as a suppository, topical contact, intravenous, intraperitoneal, intramuscular, intralesional, intranasal or subcutaneous administration, or the implantation of a slow-release device e.g., a mini-osmotic pump, to the subject. Administration is by any route including parenteral, and transmucosal (e.g., oral, nasal, vaginal, rectal, or transdermal). Parenteral administration includes, e.g., intravenous, intramuscular, intra-arteriole, intradermal, subcutaneous, intraperitoneal, intraventricular, and intracranial. Moreover, where injection is to treat a tumor, e.g., induce apoptosis, administration may be directly to the tumor and/or into tissues surrounding the tumor. Other modes of delivery include, but are not limited to, the use of liposomal formulations, intravenous infusion, transdermal patches, etc.

The term “ameliorating” or “ameliorate” refers to any indicia of success in the treatment of a pathology or condition, including any objective or subjective parameter such as abatement, remission or diminishing of symptoms or an improvement in a patient's physical or mental well-being. Amelioration of symptoms can be based on objective or subjective parameters; including the results of a physical examination and/or a psychiatric evaluation.

The term “therapy” refers to “treating” or “treatment” of a disease or condition including preventing the disease or condition from occurring in an animal that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease).

The term “effective amount” or “an amount effective to” or a “therapeutically effective amount” or any grammatically equivalent term means the amount that, when administered to an animal for treating a disease, is sufficient to effect treatment for that disease.

The term “isolated” refers to a material that is substantially or essentially free from components, which are used to produce the material. For peptide conjugates of the invention, the term “isolated” refers to material that is substantially or essentially free from components which normally accompany the material in the mixture used to prepare the peptide conjugate. “Isolated” and “pure” are used interchangeably. Typically, isolated peptide conjugates of the invention have a level of purity preferably expressed as a range. The lower end of the range of purity for the peptide conjugates is about 60%, about 70% or about 80% and the upper end of the range of purity is about 70%, about 80%, about 90% or more than about 90%.

When the peptide conjugates are more than about 90% pure, their purities are also preferably expressed as a range. The lower end of the range of purity is about 90%, about 92%, about 94%, about 96% or about 98%. The upper end of the range of purity is about 92%, about 94%, about 96%, about 98% or about 100% purity.

Purity is determined by any art-recognized method of analysis (e.g., band intensity on a silver stained gel, polyacrylamide gel electrophoresis, HPLC, or a similar means).

“Essentially each member of the population,” as used herein, describes a characteristic of a population of peptide conjugates of the invention in which a selected percentage of the modified lipids added to a peptide are added to multiple, identical acceptor sites on the peptide. “Essentially each member of the population” speaks to the “homogeneity” of the sites on the peptide conjugated to a modified lipid and refers to conjugates of the invention, which are at least about 80%, preferably at least about 90% and more preferably at least about 95% homogenous.

“Homogeneity,” refers to the structural consistency across a population of acceptor moieties to which the modified lipids are conjugated. Thus, in a peptide conjugate of the invention in which each modified lipid moiety is conjugated to an acceptor site having the same structure as the acceptor site to which every other modified lipid is conjugated, the peptide conjugate is said to be about 100% homogeneous. Homogeneity is typically expressed as a range. The lower end of the range of homogeneity for the peptide conjugates is about 60%, about 70% or about 80% and the upper end of the range of purity is about 70%, about 80%, about 90% or more than about 90%.

When the peptide conjugates are more than or equal to about 90% homogeneous, their homogeneity is also preferably expressed as a range. The lower end of the range of homogeneity is about 90%, about 92%, about 94%, about 96% or about 98%. The upper end of the range of purity is about 92%, about 94%, about 96%, about 98% or about 100% homogeneity. The purity of the peptide conjugates is typically determined by one or more methods known to those of skill in the art, e.g., liquid chromatography-mass spectrometry (LC-MS), matrix assisted laser desorption mass time of flight spectrometry (MALDITOF), capillary electrophoresis, and the like.

“Substantially uniform lipoform” or a “substantially uniform lipid pattern,” when referring to a lipopeptide species, refers to the percentage of lipid acceptor moieties to which a modified lipid is attached by the lipid transfer enzyme of interest (e.g., palmitoyltransferase). It will be understood by one of skill in the art, that the starting material may contain lipid linking groups. Thus, the calculated percent of lipids on the peptide will include acceptor moieties to which modified lipids are attached by the methods of the invention, as well as those acceptor moieties to which modified lipids are attached already in the starting material.

The term “substantially” in the above definitions of “substantially uniform” generally means at least about 40%, at least about 70%, at least about 80%, or more preferably at least about 90%, and still more preferably at least about 95% of the acceptor moieties for a particular lipid transfer enzyme are attached to a modified lipid.

All oligosaccharides described herein are described with the name or abbreviation for the non-reducing saccharide (i.e., Gal), followed by the configuration of the glycosidic bond (α or β), the ring bond (1 or 2), the ring position of the reducing saccharide involved in the bond (2, 3, 4, 6 or 8), and then the name or abbreviation of the reducing saccharide (i.e., GlcNAc). Each saccharide is preferably a pyranose. For a review of standard glycobiology nomenclature, see, Essentials of Glycobiology Varki et al. eds. CSHL Press (1999).

Oligosaccharides are considered to have a reducing end and a non-reducing end, whether or not the saccharide at the reducing end is in fact a reducing sugar. In accordance with accepted nomenclature, oligosaccharides are depicted herein with the non-reducing end on the left and the reducing end on the right.

The term “sialic acid” refers to any member of a family of nine-carbon carboxylated sugars. The most common member of the sialic acid family is N-acetyl-neuraminic acid (2-keto-5-acetamido-3,5-dideoxy-D-glycero-D-galactononulopyranos-1-onic acid (often abbreviated as Neu5Ac, NeuAc, or NANA). A second member of the family is N-glycolyl-neuraminic acid (Neu5Gc or NeuGc), in which the N-acetyl group of NeuAc is hydroxylated. A third sialic acid family member is 2-keto-3-deoxy-nonulosonic acid (KDN) (Nadano et al. (1986) J. Biol. Chem. 261: 11550-11557; Kanamori et al., J. Biol. Chem. 265: 21811-21819 (1990)). Also included are 9-substituted sialic acids such as a 9-O—C₁-C₆ acyl-Neu5Ac like 9-O-lactyl-Neu5Ac or 9-O-acetyl-Neu5Ac, 9-deoxy-9-fluoro-Neu5Ac and 9-azido-9-deoxy-Neu5Ac. For review of the sialic acid family, see, e.g., Varki, Glycobiology 2: 25-40 (1992); Sialic Acids: Chemistry, Metabolism and Function, R. Schauer, Ed. (Springer-Verlag, New York (1992)). The synthesis and use of sialic acid compounds in a sialylation procedure is disclosed in international application WO 92/16640, published Oct. 1, 1992.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain. It may include regions preceding and following the coding region (leader and trailer) as well as intervening sequences (introns) between individual coding segments (exons).

Where substituent groups are specified by their conventional chemical formulae, written from left to right, they equally encompass the chemically identical substituents, which would result from writing the structure from right to left, e.g., —CH₂O— is intended to also recite —OCH₂—.

The term “alkyl,” by itself or as part of another substituent means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups that are limited to hydrocarbon groups are termed “homoalkyl”.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, substituent that can be a single ring or multiple rings (preferably from 1 to 3 rings), which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, tetrazolyl, benzo[b]furanyl, benzo[b]thienyl, 2,3-dihydrobenzo[1,4]dioxin-6-yl, benzo[1,3]dioxol-5-yl and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) is meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′ R″, —OC(O)NR′R″, —NR″C(O)R′, —NR—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are generically referred to as “aryl group substituents.” The substituents are selected from, for example: halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′ R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, substituted or unsubstituted alkyl, substituted or unsubstituted heteroalkyl, substituted or unsubstituted aryl and substituted or unsubstituted heteroaryl. When a compound of the invention includes more than one R group, for example, each of the R groups is independently selected as are each R′, R″, R′″ and R″″ groups when more than one of these groups is present. In the schemes that follow, the symbol X represents “R” as described above.

Two of the substituents on adjacent atoms of the aryl or heteroaryl ring may optionally be replaced with a substituent of the formula -T-C(O)—(CRR′)ᵤ—U—, wherein T and U are independently —NR—, —O—, —CRR′— or a single bond, and u is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)_{z}—X—(CR″R′″)_{d}—, where z and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

As used herein, the term “heteroatom” is meant to include oxygen (O), nitrogen (N), sulfur (S), silicon (Si) and phosphorus (P).

### III. Introduction

The present invention provides conjugates between peptides and modifying groups attached through a lipid-based linker moiety. The lipids may be attached to a glycosyl residue and/or an amino acid residue of a peptide. Also provided are enzymatically-mediated methods for producing the peptide conjugates of the invention. The invention also provides pharmaceutical formulations that include a peptide conjugate formed by a method of the invention.

The therapeutic peptide conjugates of the invention are formed between a therapeutic core molecule, e.g., a peptide, and diverse species such as water-soluble polymers, therapeutic moieties, diagnostic moieties, targeting moieties and the like. Also provided are conjugates that include two or more peptides linked together through a linker arm, i.e., multifunctional conjugates. The multi-functional conjugates of the invention can include two or more copies of the same peptide or a collection of diverse peptides with different structures and/or properties. In exemplary conjugates according to this embodiment, the linker between the two peptides is attached to at least one of the peptides through a lipid linking group.

The conjugates of the invention are prepared by the enzymatic conjugation of a modifying group to a lipid moiety, forming a ‘modified lipid’. When the conjugate of the invention is a peptide conjugate, the modified lipid is attached directly to an amino acid of a peptide comprising the lipid modification recognition site of that peptide.

The modified lipid, when interposed between the peptide and the modifying group becomes what is referred to herein as a “lipid linking group”. Using the exquisite selectivity of enzymes, such as prenyltransferases, farnesyltransferases, myristoyltransferases, and palmitoyltransferases the present method provides peptides that bear a desired group at one or more specific locations. Thus, in exemplary conjugates according to the present invention, a modified lipid is attached directly to a selected locus on the peptide chain.

The methods of the invention make it possible to assemble modified peptides that have a substantially homogeneous derivatization pattern; the enzymes used in the invention are generally selective for a particular glycosyl residue, amino acid residue or for particular substituents, or substituent patterns, on an amino acid residue. The methods are also practical for large-scale production of modified lipopeptide conjugates. In one embodiment, the methods of the invention provide a practical means for large-scale preparation of lipopeptide conjugates having preselected uniform derivatization patterns. The methods are particularly well suited for modification of therapeutic peptides.

The methods of the invention also provide therapeutic peptide conjugates with increased therapeutic half-life due to, for example, reduced clearance rate, or reduced rate of uptake by the immune or reticuloendothelial system (RES). Selective attachment of targeting agents to a peptide using an appropriate modified lipid can be used to target the peptide or to a particular tissue or cell surface receptor that is specific for the particular targeting agent. Finally, there is provided a class of peptides that are specifically modified with a therapeutic moiety conjugated through a lipid linking group.

### IV. The Embodiments

The peptide conjugates of the invention will typically correspond to the following general structure:

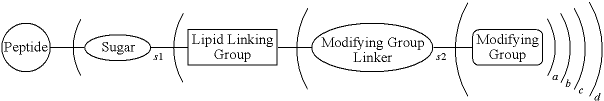

in which the symbols a, b, c and d represent a positive, non-zero integer; and s1 and s2 are either 0 or a positive integer. The “modifying group” is a therapeutic agent, a bioactive agent, a detectable label, water-soluble polymer (e.g., PEG, m-PEG, PPG, and m-PPG), water-insoluble polymer or the like. The “modifying group” can be a peptide, e.g., enzyme, antibody, antigen, etc. The modifying group linker can be any of a wide array of linking groups, infra. Alternatively, the modifying group linker may be a single bond or a “zero order linker.”

### IV a) The Compositions of Matter/Peptide Conjugates

The present invention provides peptide conjugates in which the peptide is conjugated to a modifying group through a lipid linking group and optionally through a sugar and/or modifying group linker. In one aspect, the invention provides a peptide conjugate including a peptide and a modified lipid, in which the modified lipid comprises at least one lipid linking group and at least one modifying group. The modifying group is covalently attached to the peptide at a preselected glycosyl and/or amino acid residue of the peptide via a lipid linking group. In an exemplary embodiment, the peptide and the modified lipid are linked through an atom on the side chain of an amino acid residue of the peptide, and this atom is a member selected from oxygen, sulfur and nitrogen. In another exemplary embodiment, the peptide and the modified lipid are linked through an atom at a terminus of the peptide, and this atom is a member selected from oxygen and nitrogen.

### IV. b) Peptide

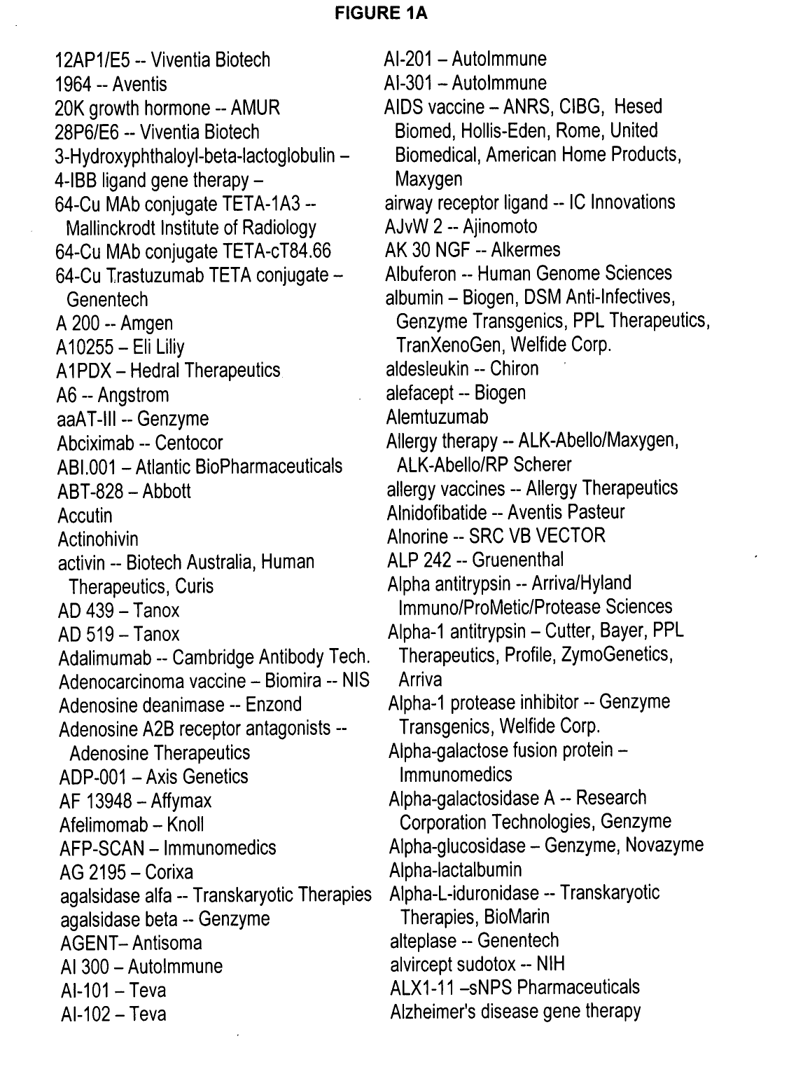

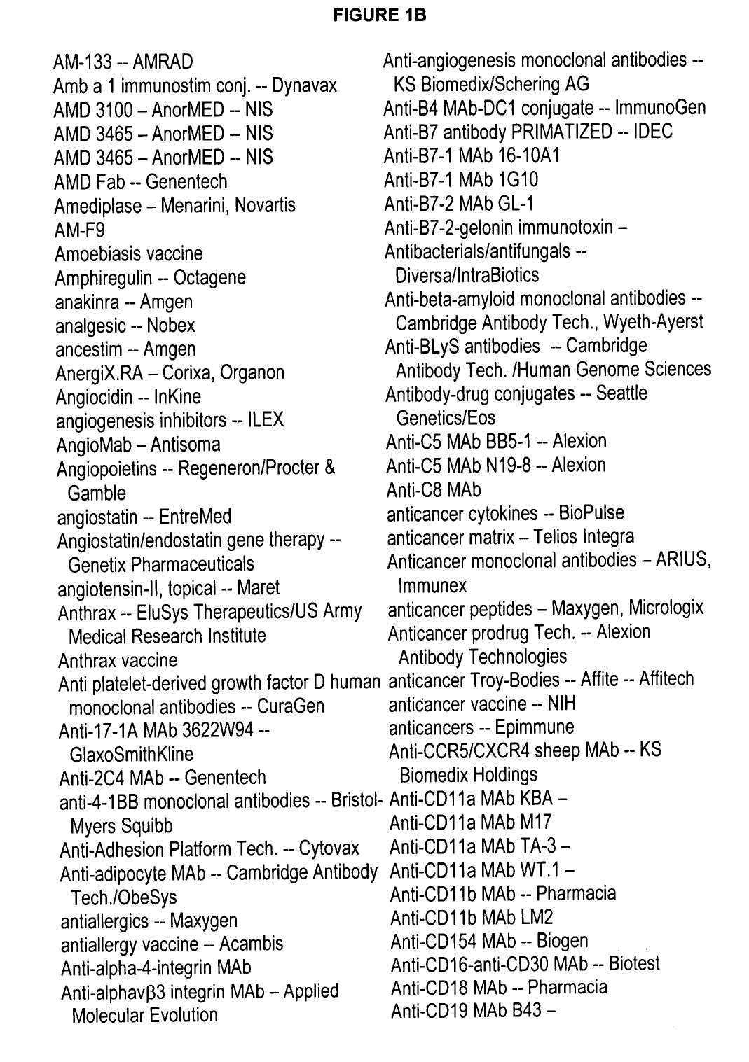

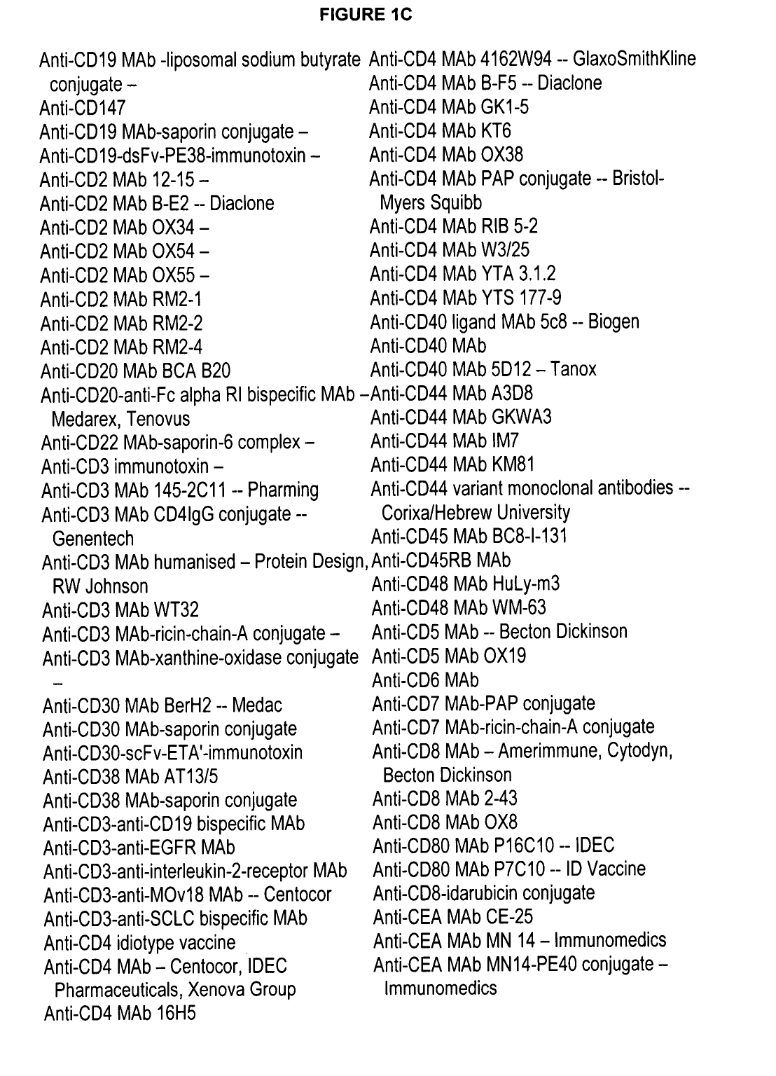

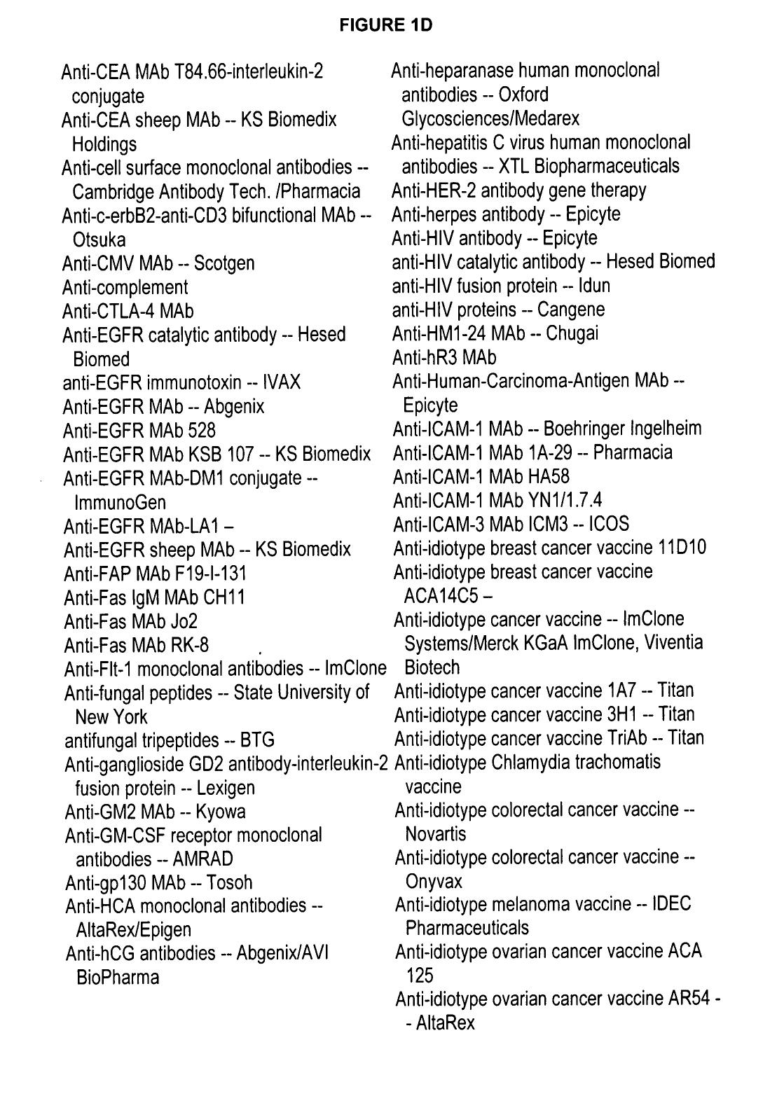

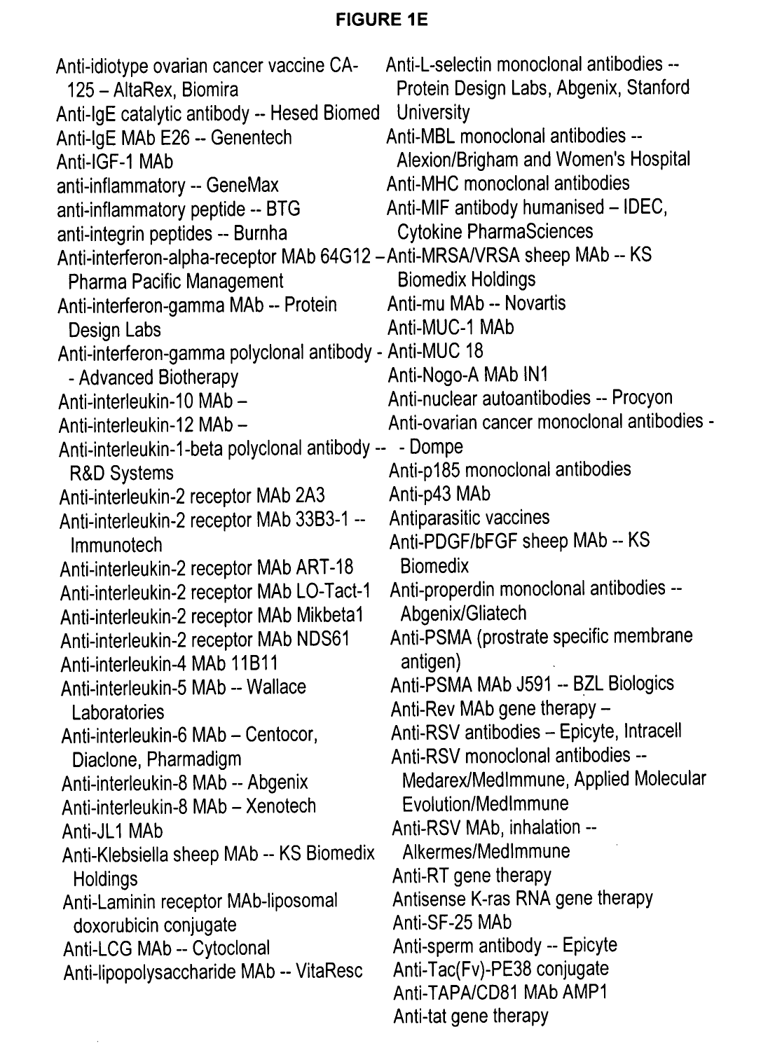

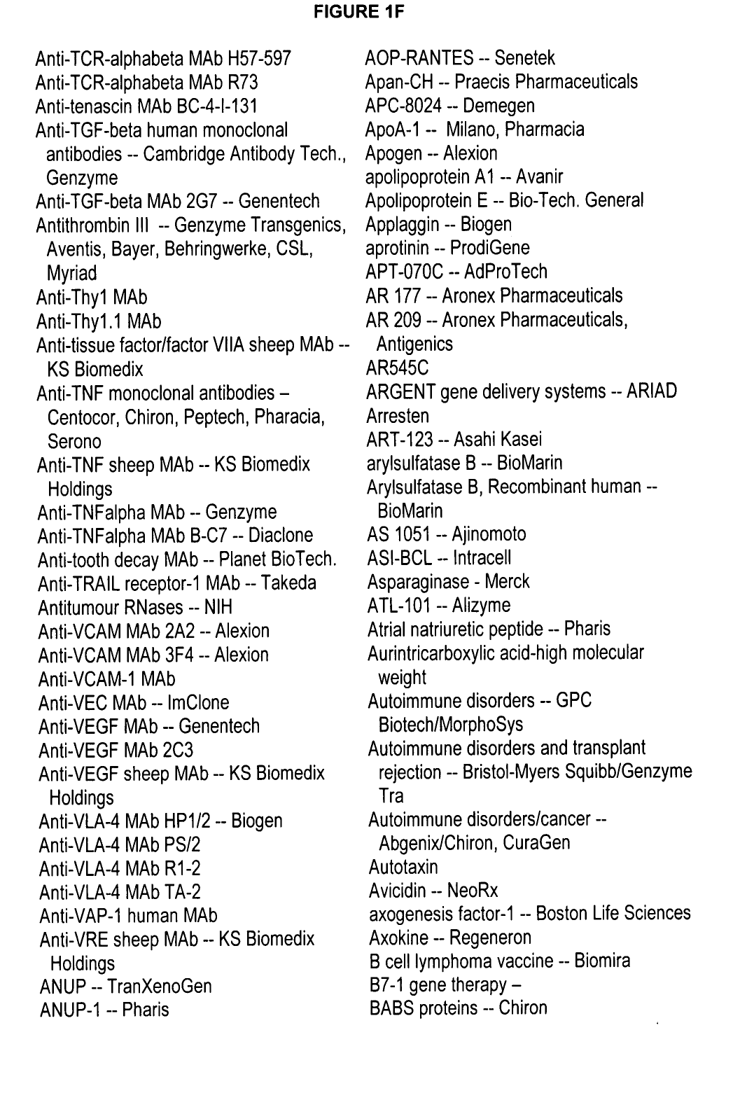

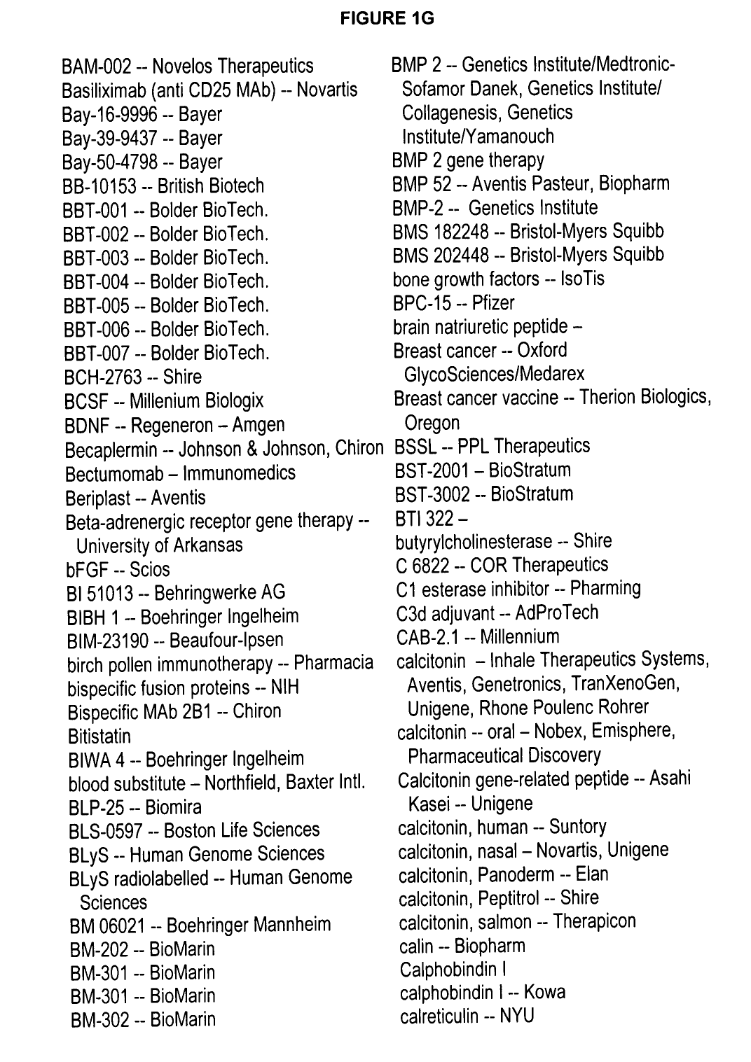

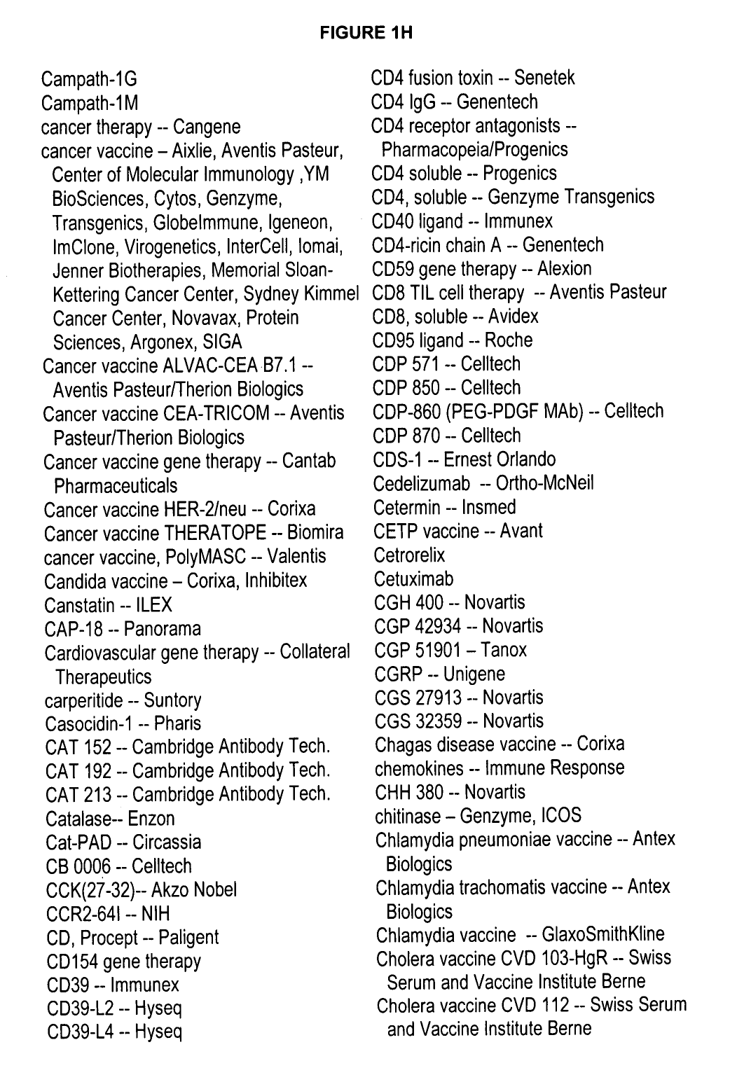

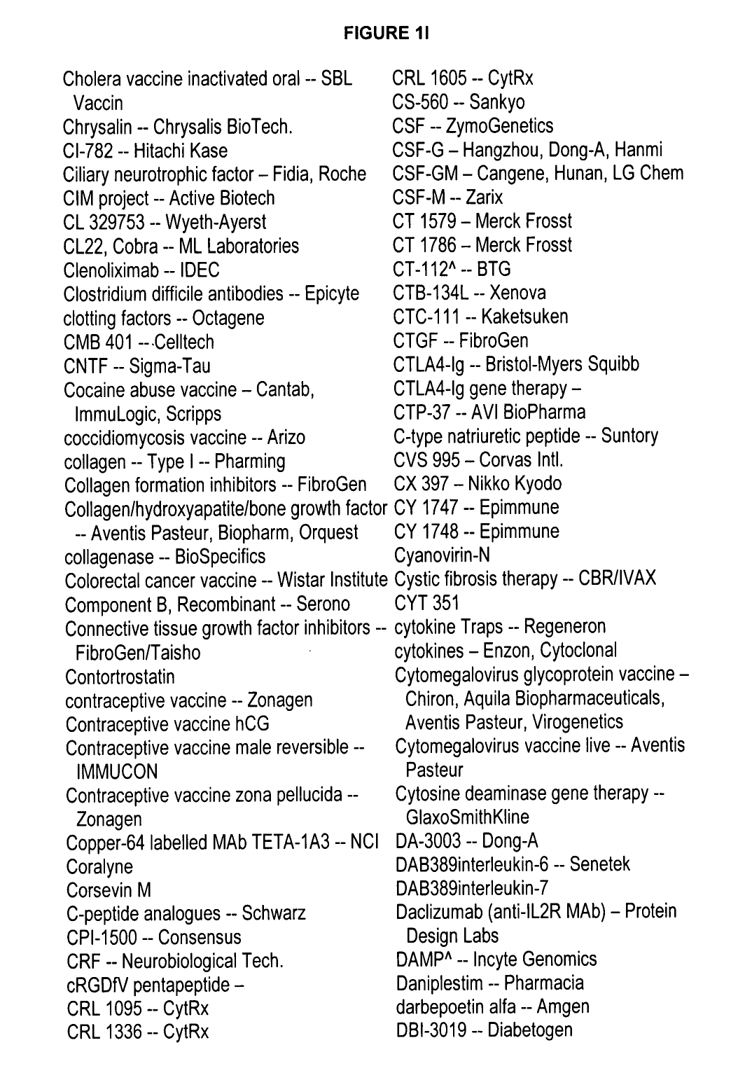

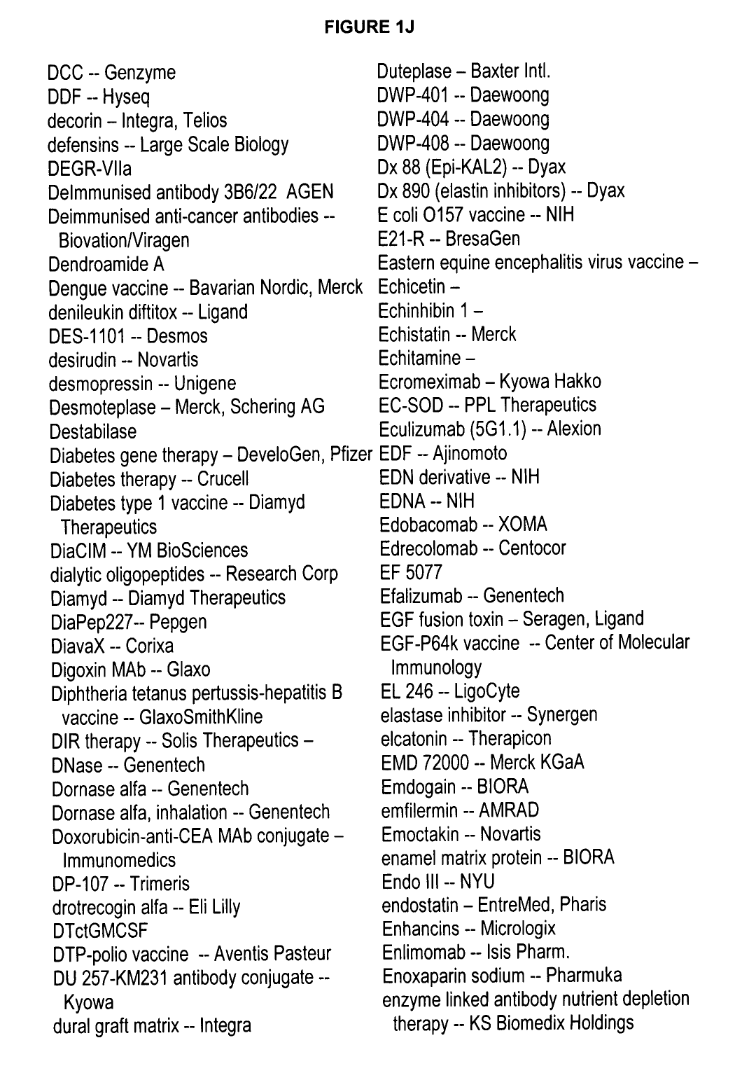

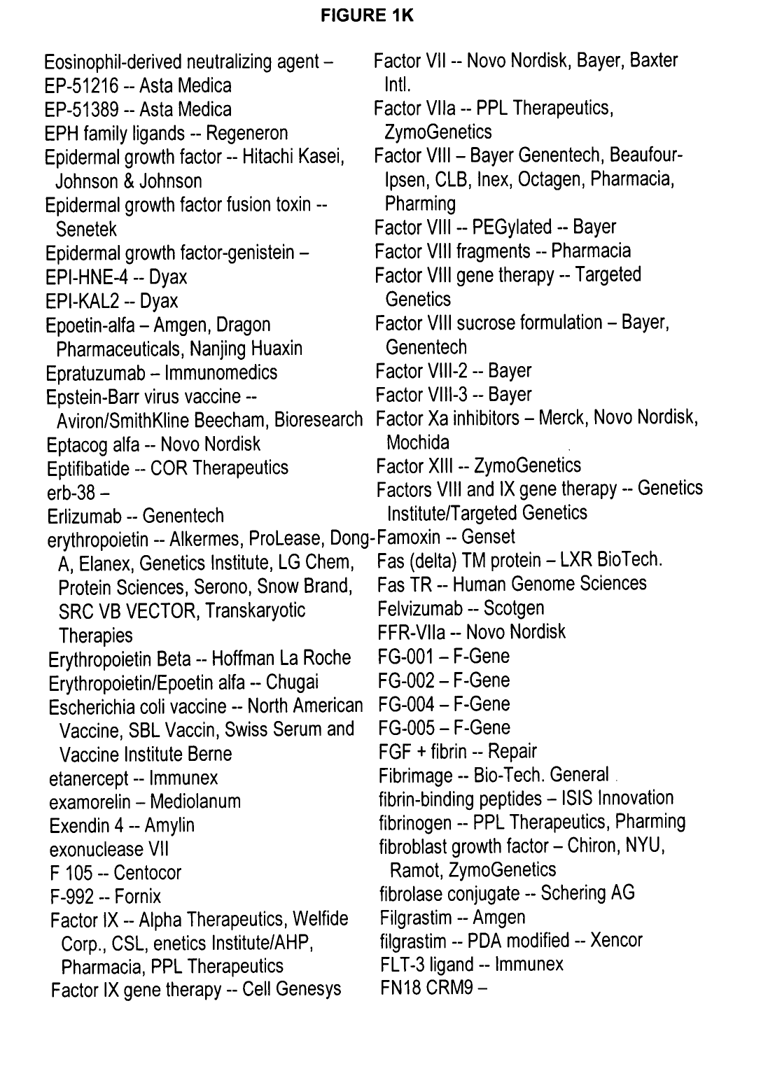

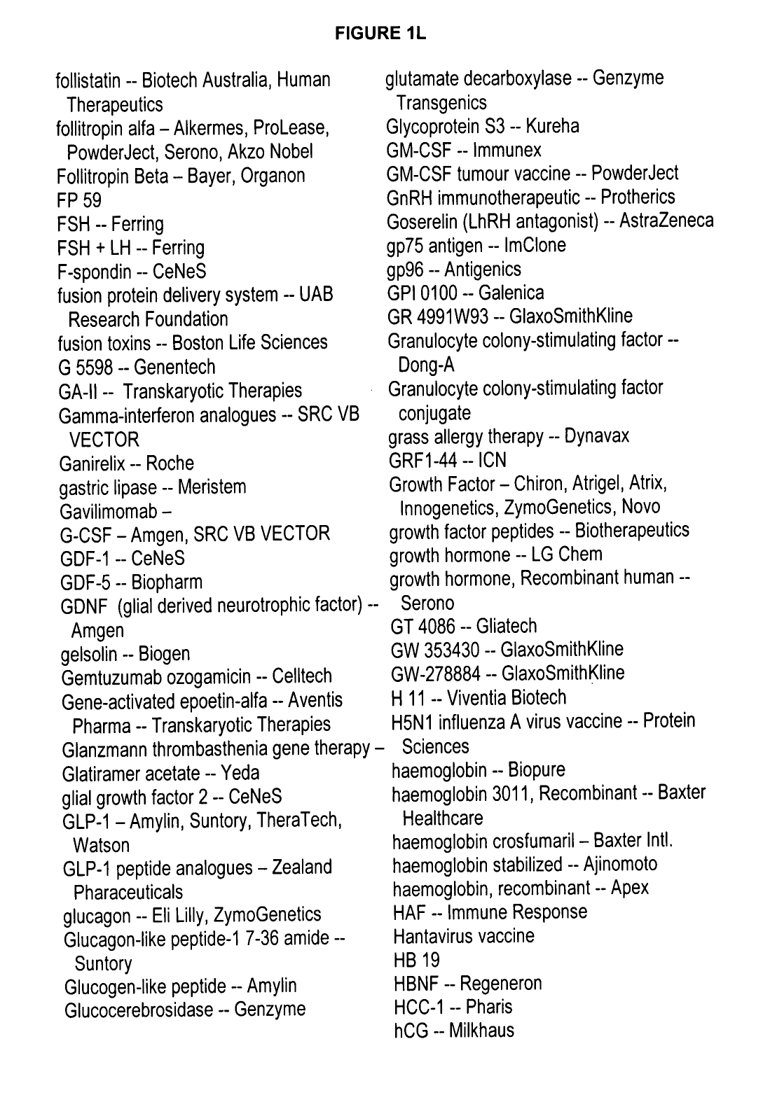

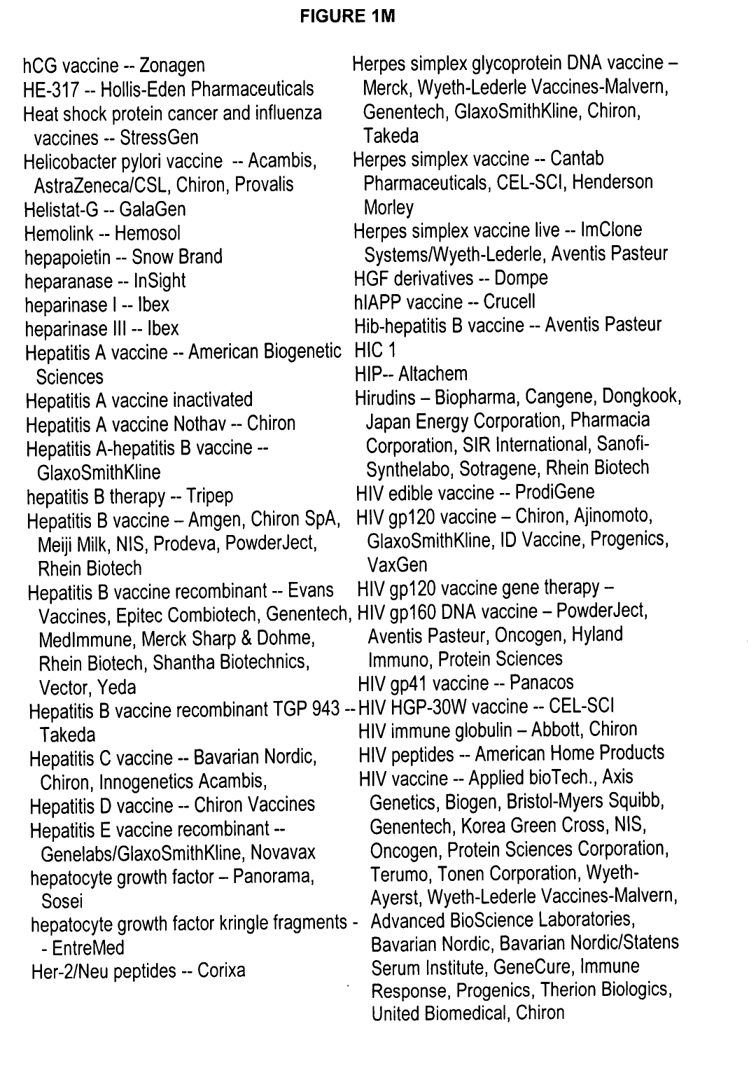

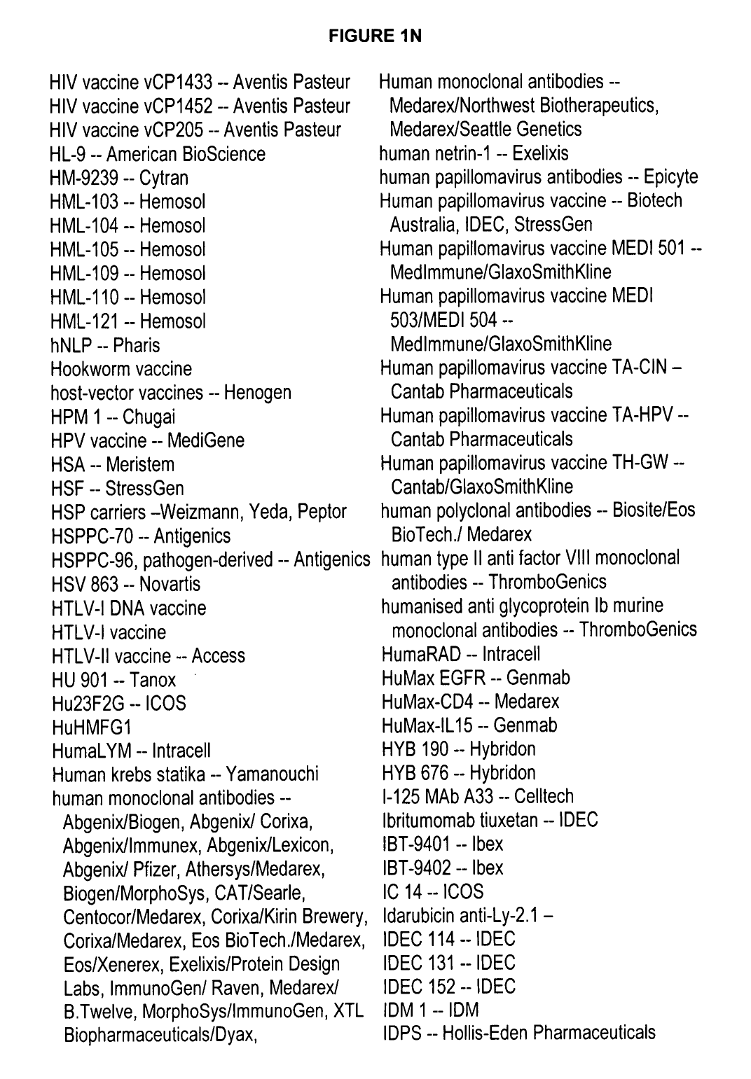

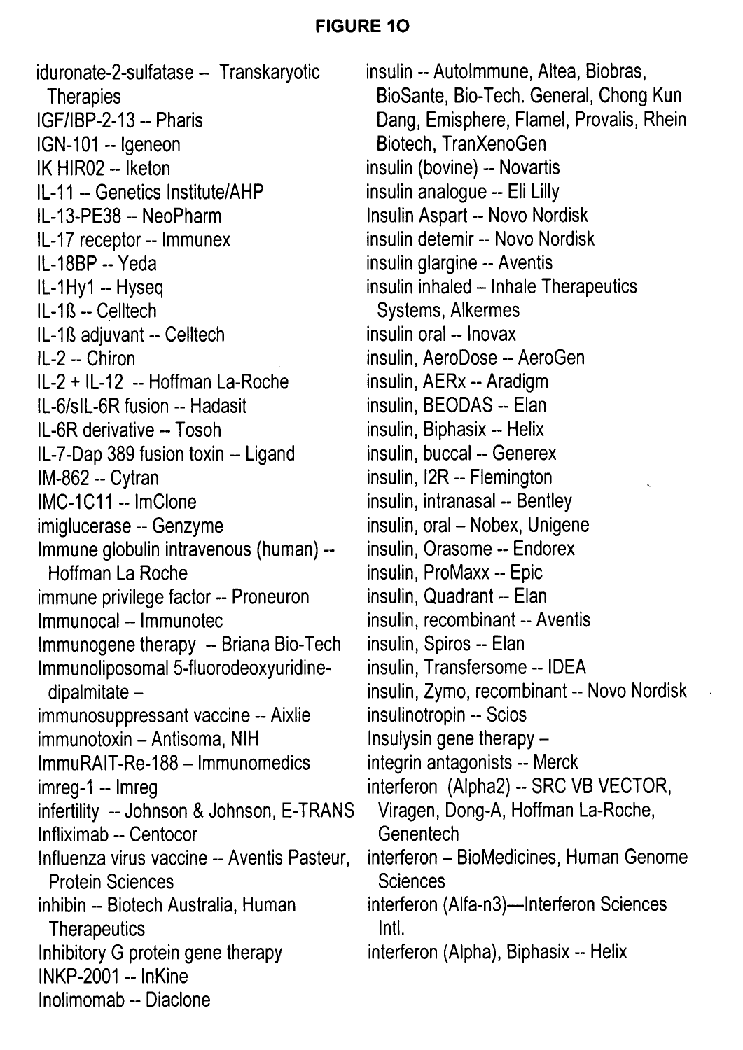

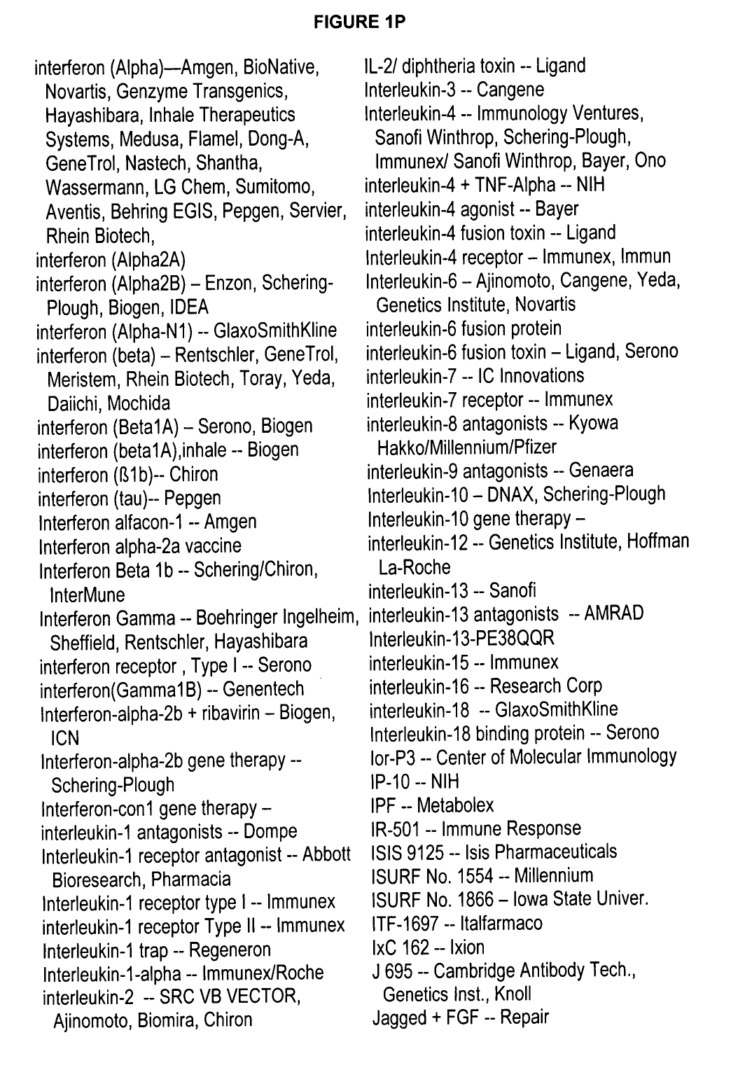

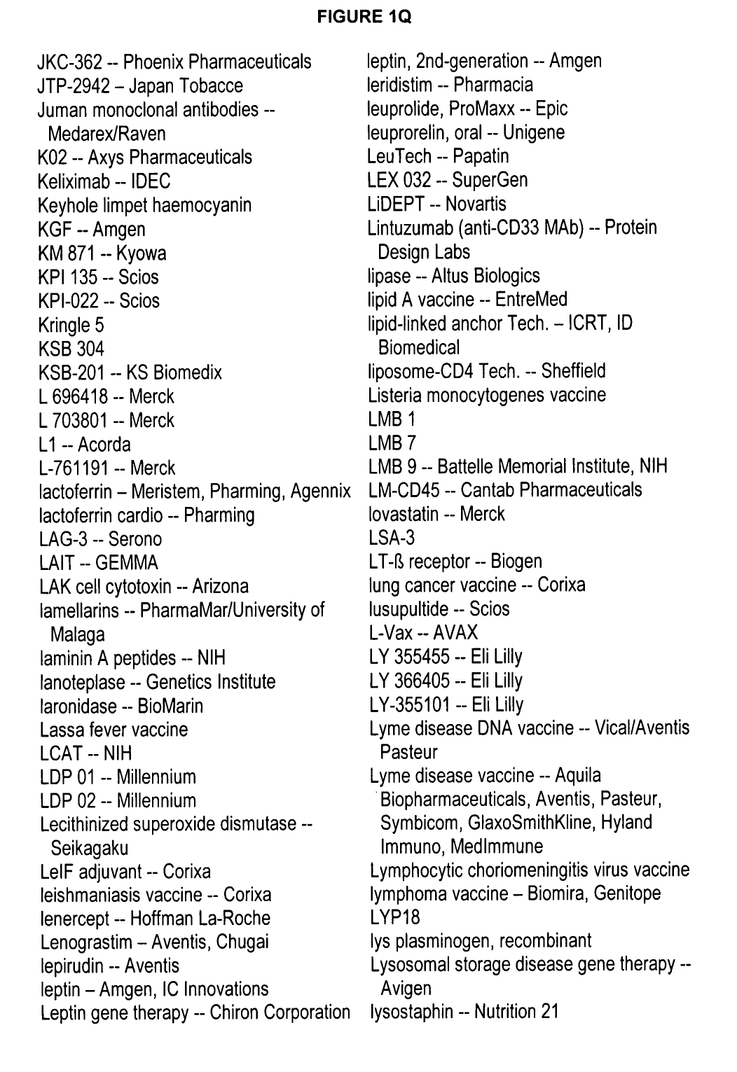

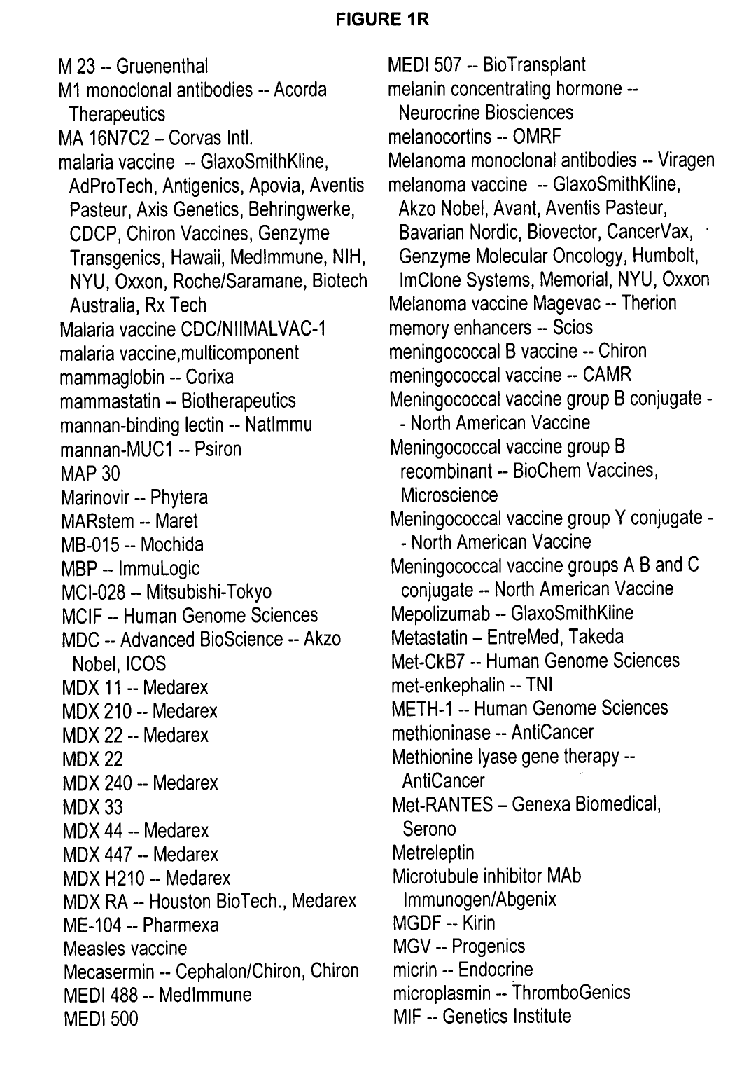

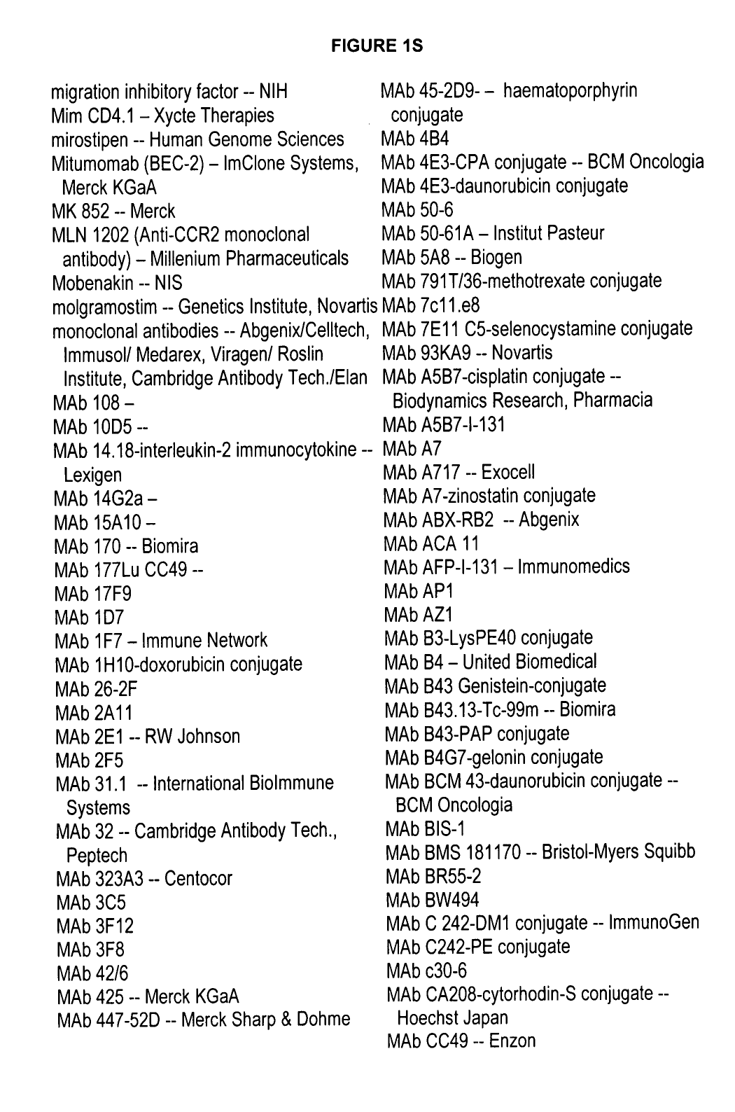

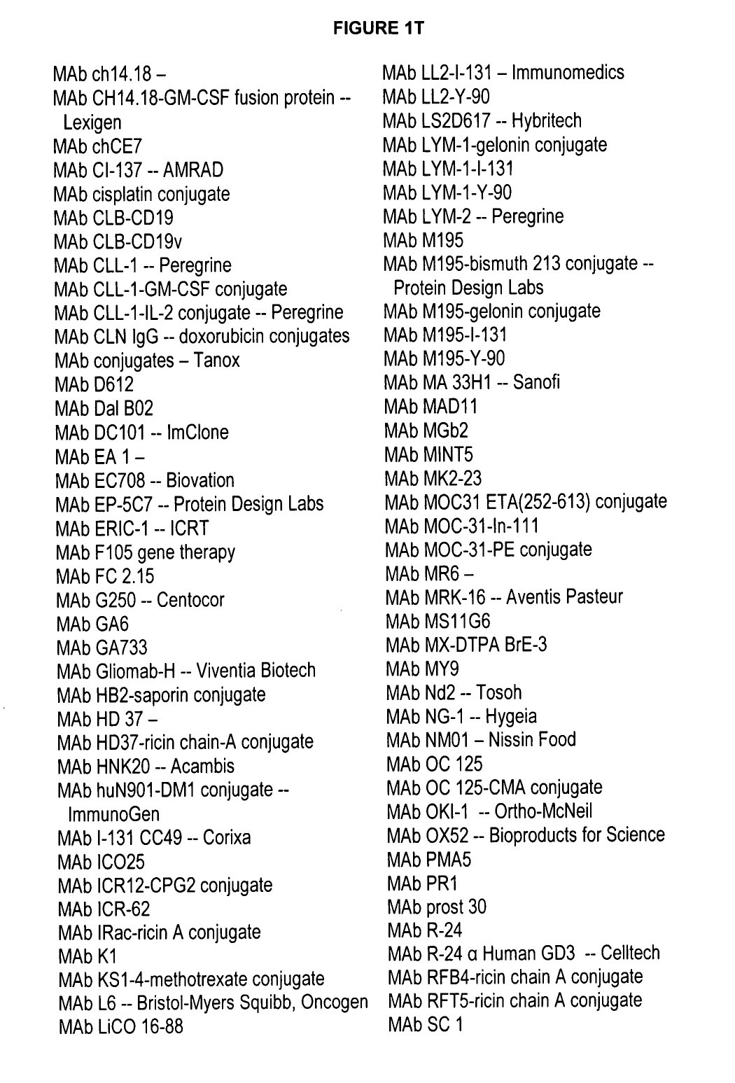

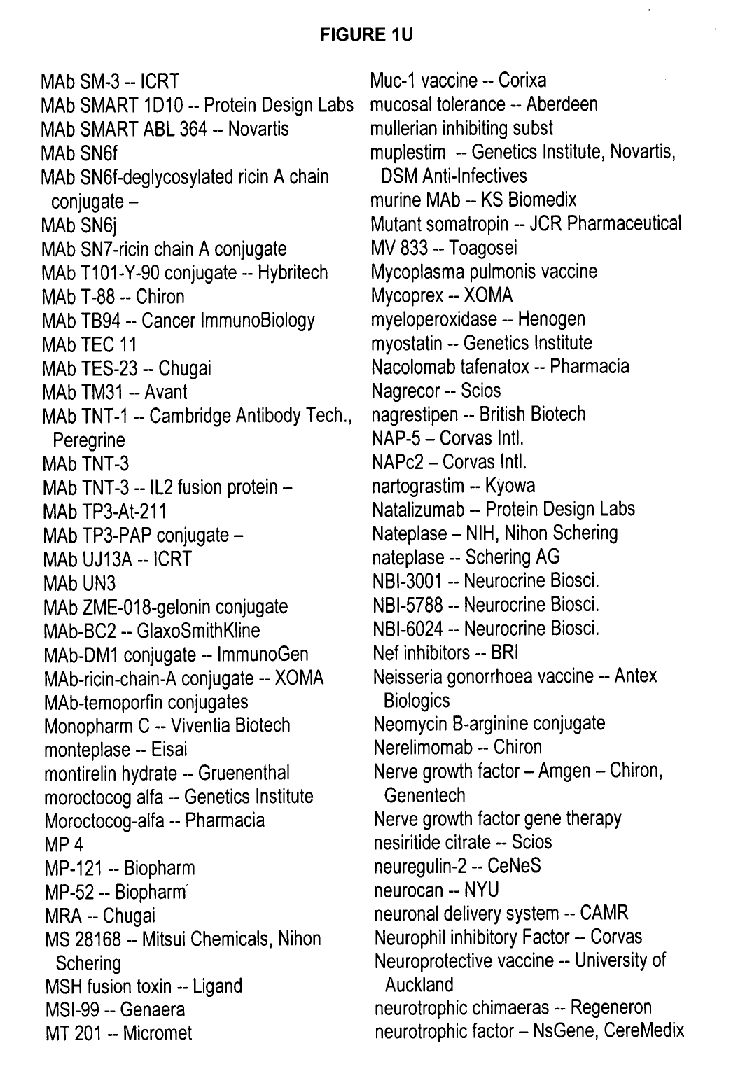

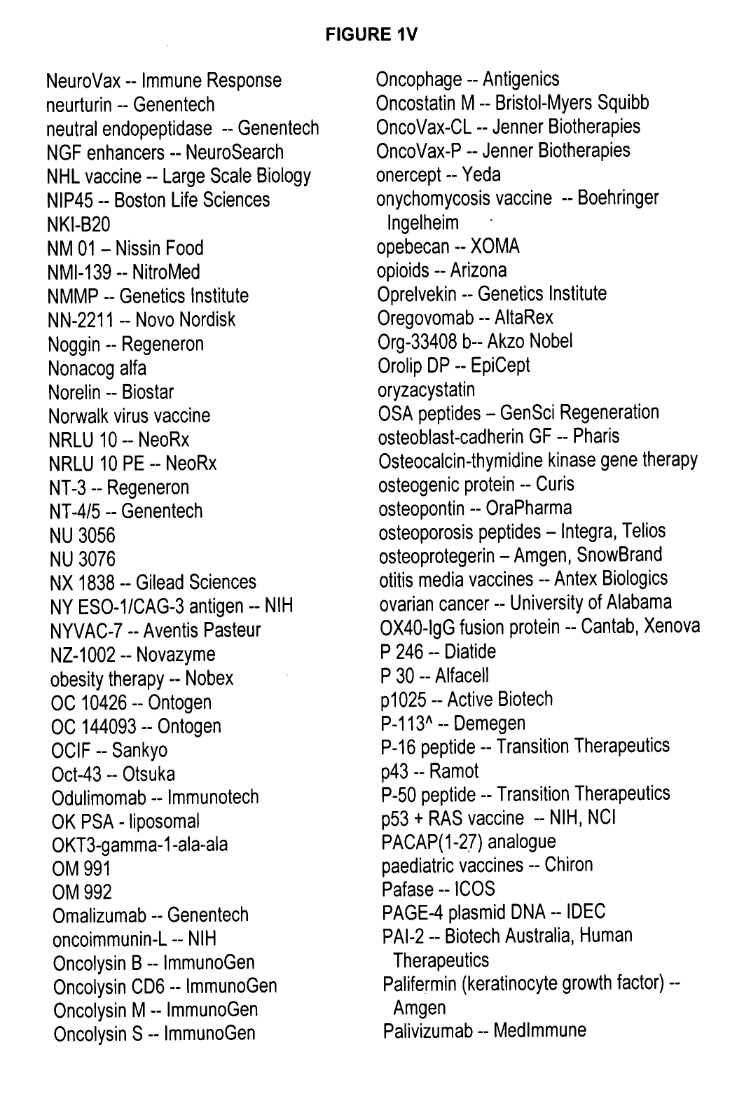

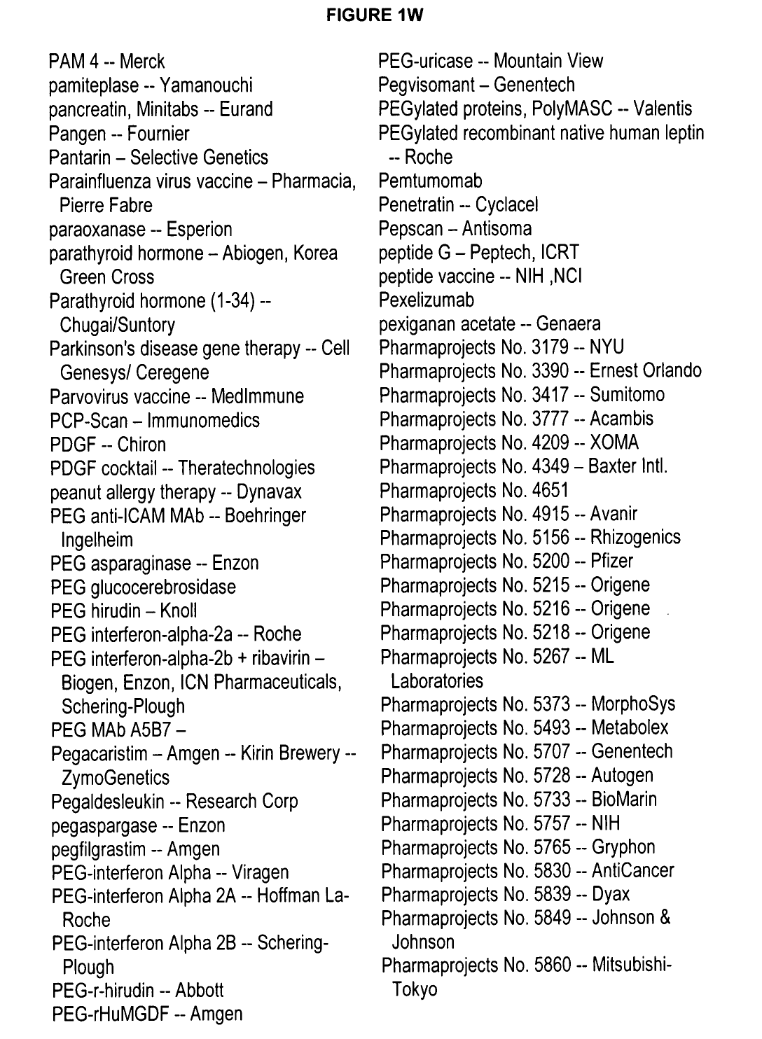

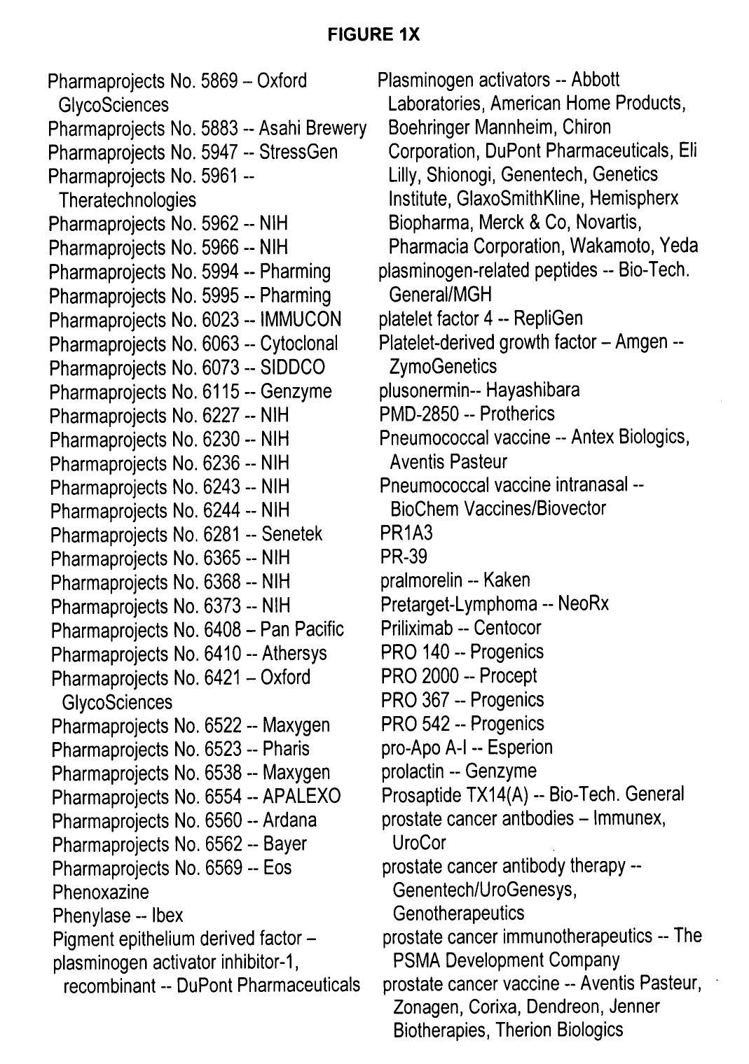

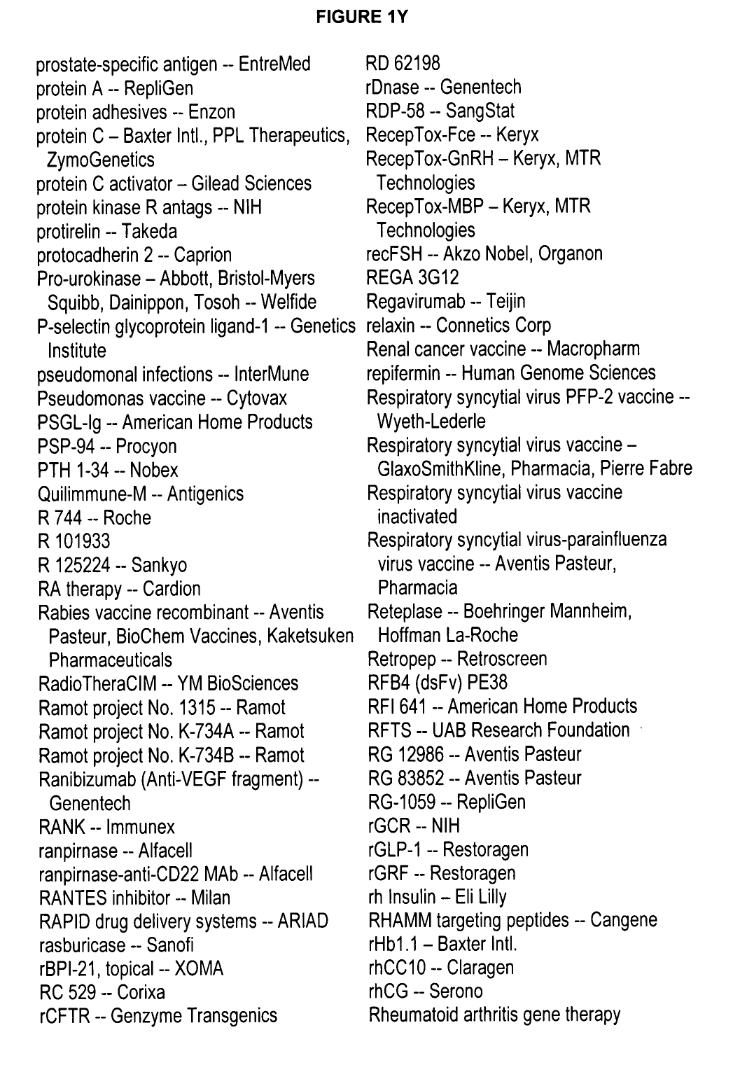

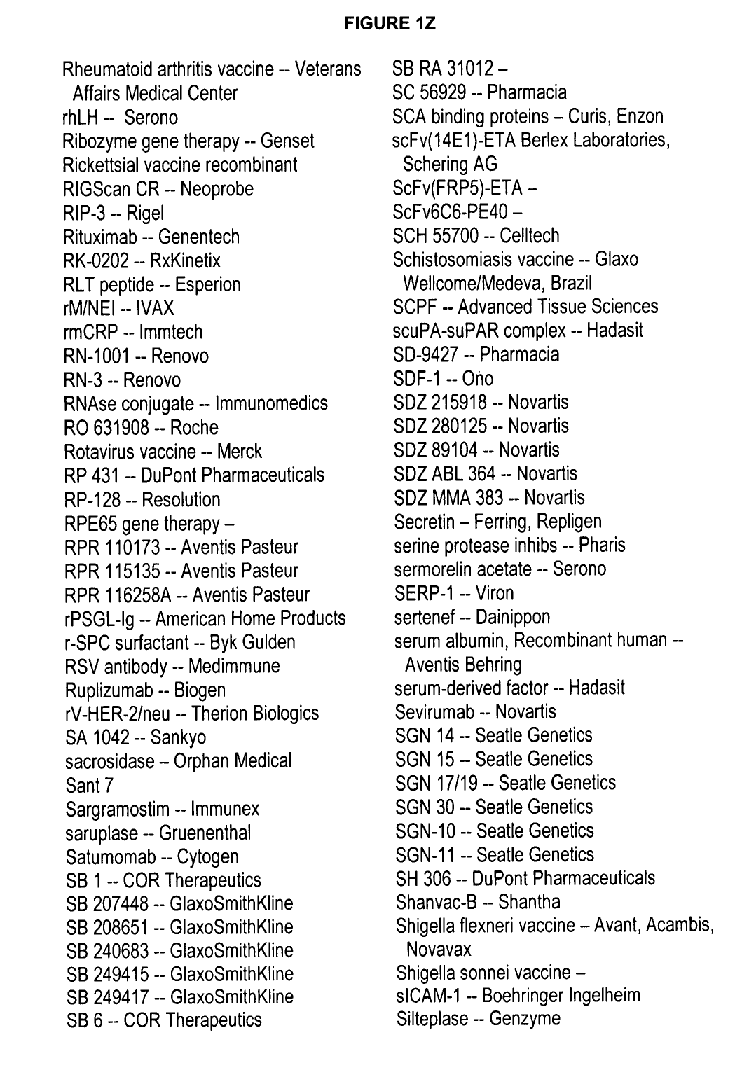

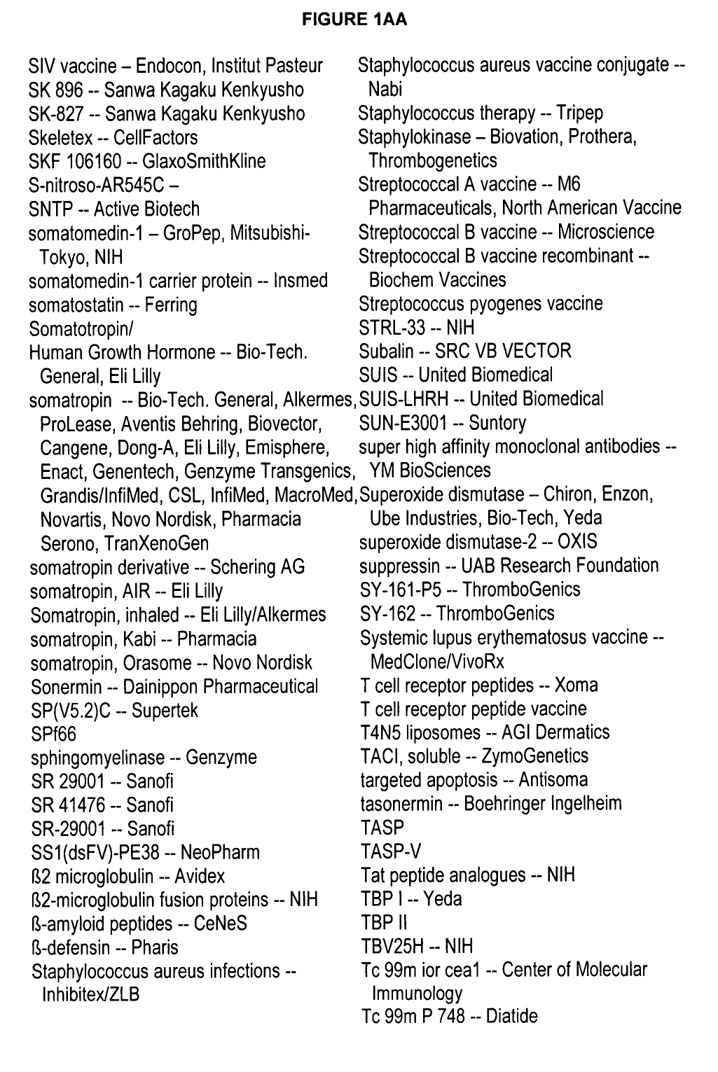

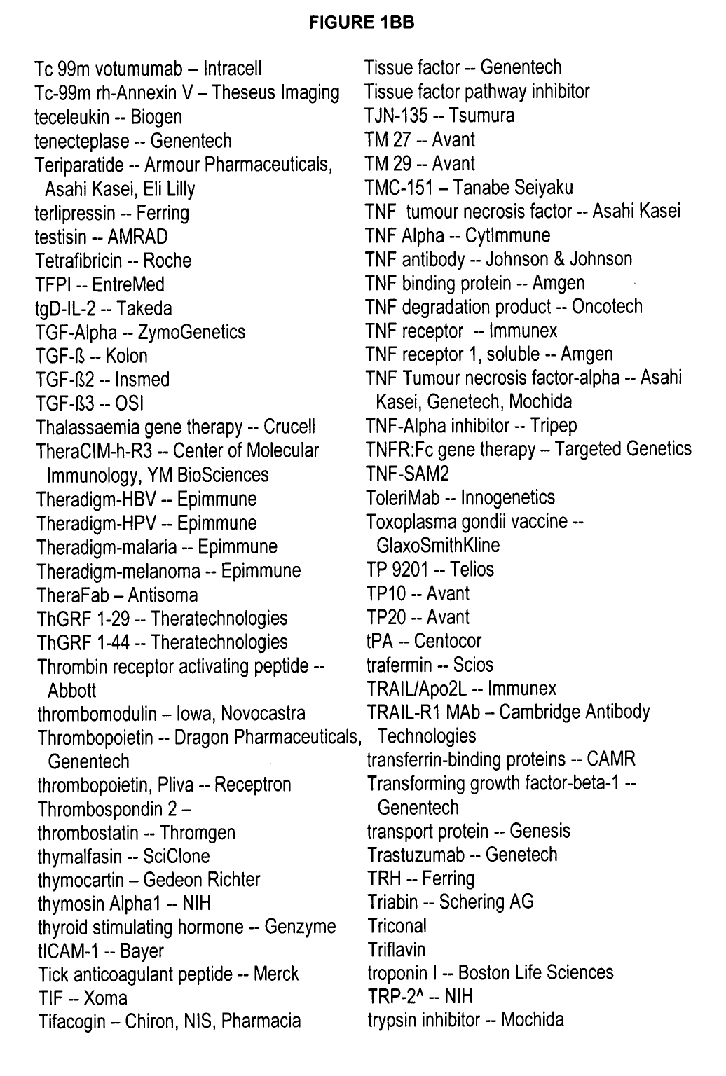

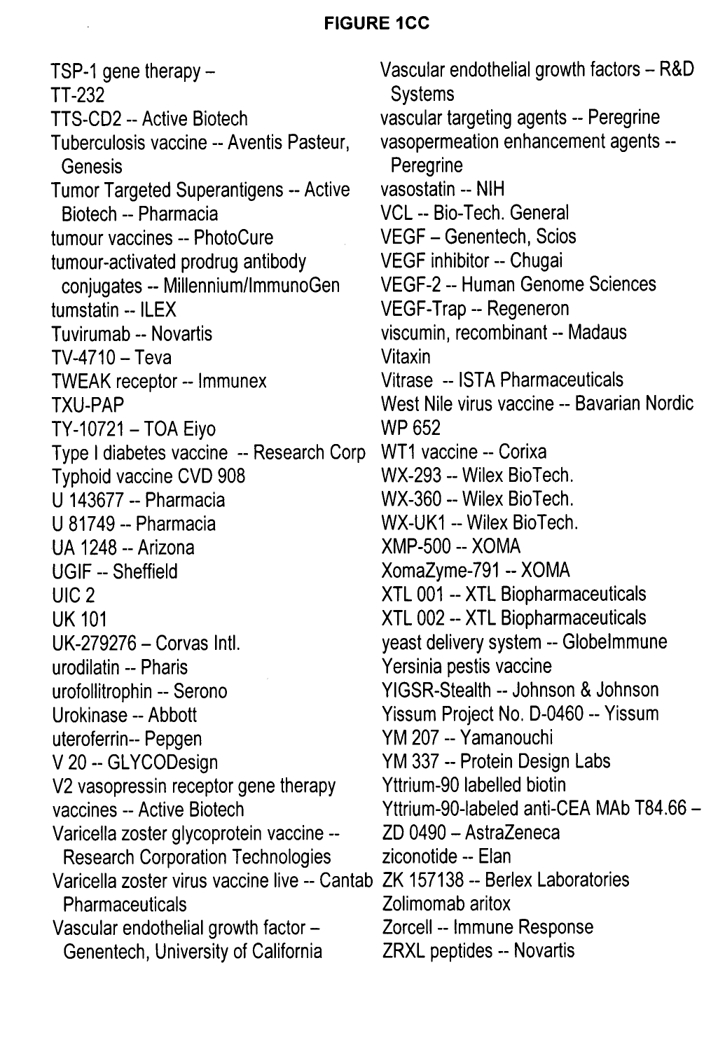

The peptide conjugates of the invention encompasses the use of almost any peptide. A description of exemplary peptides is provided in FIG. 1. In an exemplary embodiment, peptides include members of the immunoglobulin family (e.g., antibodies, MHC molecules, T cell receptors, and the like), intercellular receptors (e.g., integrins, receptors for hormones or growth factors and the like) lectins, and cytokines (e.g., interleukins). In another exemplary embodiment, the peptide is a member selected from clotting factors such as Factor V, Factor VI, Factor VII, Factor VIIa, Factor VIII, Factor IX, Factor X, Factor XI, and Factor XII, bombesin, thrombin, hematopoietic growth factor, colony stimulating factors, viral antigens, complement proteins, erythropoietin, granulocyte colony stimulating factor (G-CSF), Granulocyte-Macrophage Colony Stimulating Factor (GM-CSF), interferons, interferon alpha, interferon beta, interferon gamma, α₁-antitrypsin (ATT, or α-1 protease inhibitor, glucocerebrosidase, Tissue-Type Plasminogen Activator (TPA), renin, P-selectin glycopeptide ligand-1 (PSGL-1), interleukins, Interleukin-2 (IL-2), urokinase, human DNase, proteins A and C, fibrinogen, herceptin, leptin, glycosidases, HS-glycoprotein, serum proteins (e.g., α-acid glycoprotein, fetuin, α-fetal protein), β2-glycoprotein, insulin, Hepatitis B surface protein (HbsAg), human growth hormone, TNF Receptor-IgG Fc region fusion protein (Enbrel™), anti-HER2 monoclonal antibody (Herceptin™), monoclonal antibody to Protein F of Respiratory Syncytial Virus (Synagis™), monoclonal antibody to TNF-α (Remicade™), monoclonal antibody to glycoprotein IIb/IIIa (Reopro™), monoclonal antibody to CD20 (Rituxan™), anti-thrombin III (AT III), human Chorionic Gonadotropin (hCG), alpha-galactosidase (Fabrazyme™), alpha-iduronidase (Aldurazyme™), follicle stimulating hormone, beta-glucosidase, anti-TNF-alpha monoclonal antibody, glucagon-like peptide-1 (GLP-1), beta-glucosidase, alpha-galactosidase A and fibroblast growth factor. The exemplary peptides provided herein are intended to provide a selection of the peptides with which the present invention can be practiced; as such, they are non-limiting. Those of skill will appreciate that the invention can be practiced using substantially any peptide from any source.

Peptides modified by the methods of the invention can be synthetic or wild-type peptides or they can be mutated peptides, produced by methods known in the art, such as site-directed mutagenesis.

### IV. c) Lipid Linking Group

In an exemplary embodiment, the lipid linking group is a member selected from

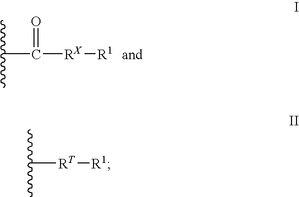

in which R^{X} is a member selected from substituted or unsubstituted, saturated or unsaturated C₁-C₄₀ alkyl, and [symbol] describes the point of attachment between the lipid linking group and the peptide. R^{T} includes at least one moiety which has a structure according to the formula:

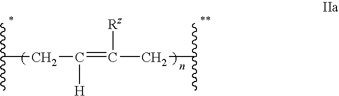

in which R^{z} is a member selected from H and substituted or unsubstituted methyl, and [symbol] and [symbol] describe the points of attachment between said moiety and the remainder of the main chain of the modified lipid. The index n is an integer selected from 1 to 20.

In an exemplary embodiment, the modified lipid has a formula according to Formula II, and said Formula II is a member selected from:

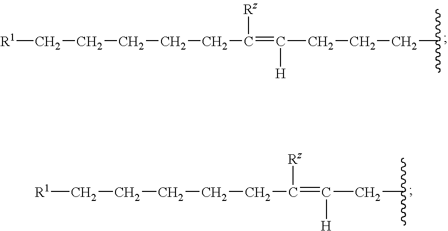

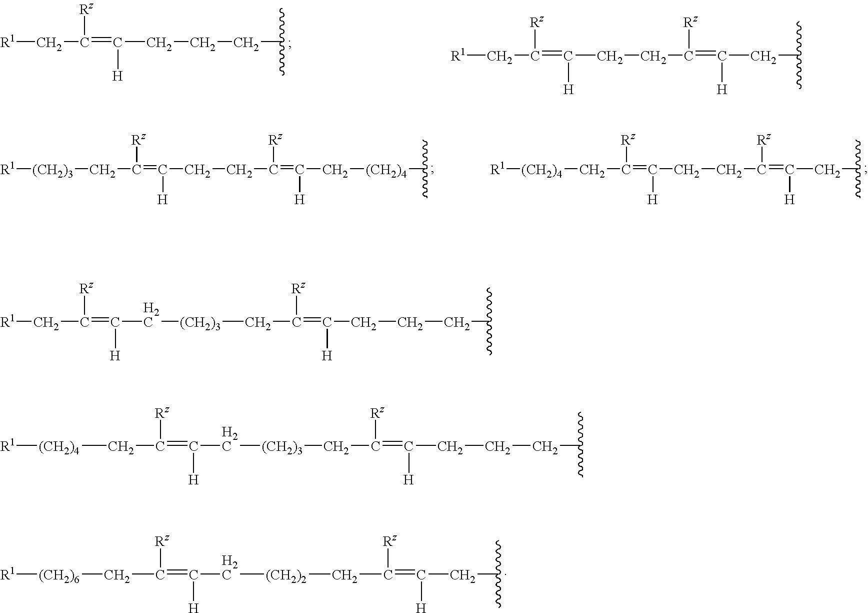

In an exemplary embodiment, the lipid linking group is

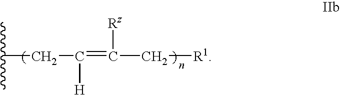

In an exemplary embodiment, the index n is an integer from 1 to 6. In yet another exemplary embodiment, n is 3. In another exemplary embodiment, n is 4. In another exemplary embodiment, at least one of the lipid linking groups is conjugated to the peptide through a sulfur atom on one or more cysteine residues near the C-terminal end of the protein. In another exemplary embodiment, at least one of the lipid linking groups is conjugated to a single C-terminal cysteine residue that is embedded within a C-terminal amino acid sequence of CAAX. In CAAX, A can be any aliphatic amino acid, and X is a member selected from methionine, glutamine, serine and leucine. In another exemplary embodiment, n is 3 and X is a member selected from methionine, glutamine and serine. In another exemplary embodiment, n is 4 and X is leucine. In yet another exemplary embodiment, at least two lipid linking groups are conjugated separately to two cysteine residues, and the cysteine residues are embedded within a C-terminal amino acid sequence which is a member selected from: cysteine-cysteine and cysteine-X²-cysteine, in which X² is any amino acid.

In an exemplary embodiment, the lipid linking group is

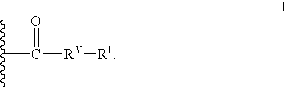

In an exemplary embodiment, R^{X} is a member selected from substituted or unsubstituted, saturated or unsaturated C₁₀-C₁₈. In another exemplary embodiment, R^{X} is a member selected from substituted or unsubstituted, saturated or unsaturated C₁₄-C₁₈ alkyl. R^{X} is a member selected from substituted or unsubstituted, saturated or unsaturated C₁₄ alkyl. In another exemplary embodiment, at least one of said lipid linking groups is conjugated to the peptide through a thioester linkage with one or more cysteine residues of the peptide. In yet another exemplary embodiment, the peptide comprises a first cysteine residue, and the first cysteine residue is near one of the peptide termini. In still another exemplary embodiment, the first cysteine residue is near the C-terminus of the peptide, and the peptide further includes a second cysteine residue, and the second cysteine residue is conjugated through a sulfur atom on one or more cysteine residues to a lipid linking group having a structure according to the following formula:

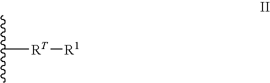

in which the first cysteine residue is internal relative to the second cysteine residue. In yet another exemplary embodiment, the first cysteine residue is near the N-terminus of the peptide, and the peptide is further conjugated through a nitrogen atom at its N-terminus to a lipid linking group having a structure according to the following formula:

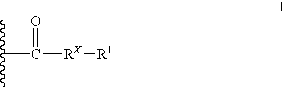

in which the first cysteine residue near the N-terminus of the peptide is internal relative to the N-terminus. In another exemplary embodiment, the lipid linking group is attached to the peptide through a nitrogen atom. R^{X} is a member selected from substituted or unsubstituted, saturated or unsaturated C₁₂-C₁₈ alkyl. In another exemplary embodiment, the lipid linking group is conjugated to the peptide through a nitrogen atom on an N-terminal glycine residue.

In another exemplary embodiment, the N-terminal glycine residue is located at position 2 of the peptide sequence.

The invention provides a peptide conjugate that includes a lipid linking group which is attached to a glycosyl residue of the peptide with a structure which is a member selected from the following formulas:

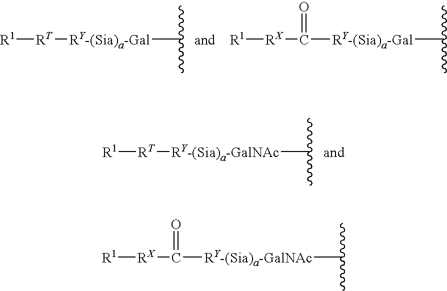

in which the index a is 0 or 1 and R^{Y} is a member selected from a bond, O, S and NH.

In other embodiments, the lipid linking group is attached to a glycosyl residue of the peptide and has a structure which is a member selected from the following formulas:

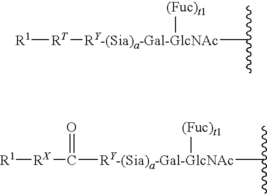

in which the index a is 0 or 1, the index t1 is 0 or 1 and R^{Y} is a member selected from a bond, O, S and NH.

In a still further exemplary embodiment, the lipid linking group is attached to a glycosyl residue of the peptide and has the formula:

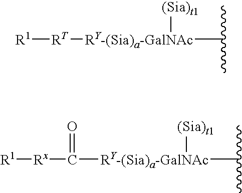

in which the index a is 0 or 1 and the index t1 is 0 or 1.

In yet another embodiment, the lipid linking group has the formula:

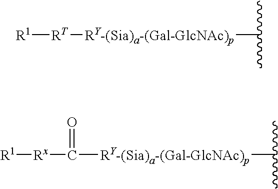

in which the index p represents an integer from 1 to 10 and R^{Y} is a member selected from a bond, O, S and NH.

In another exemplary embodiment, the peptide conjugate comprises a lipid moiety selected from the formulae:

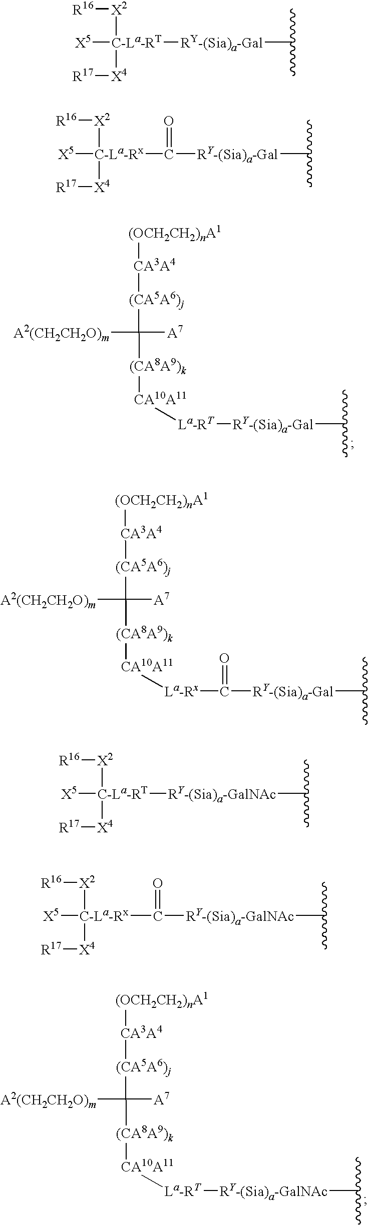

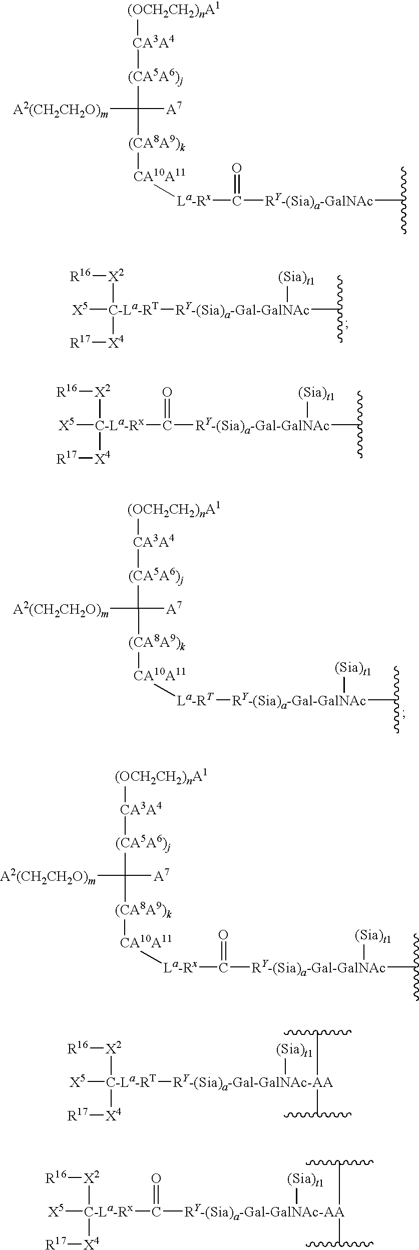

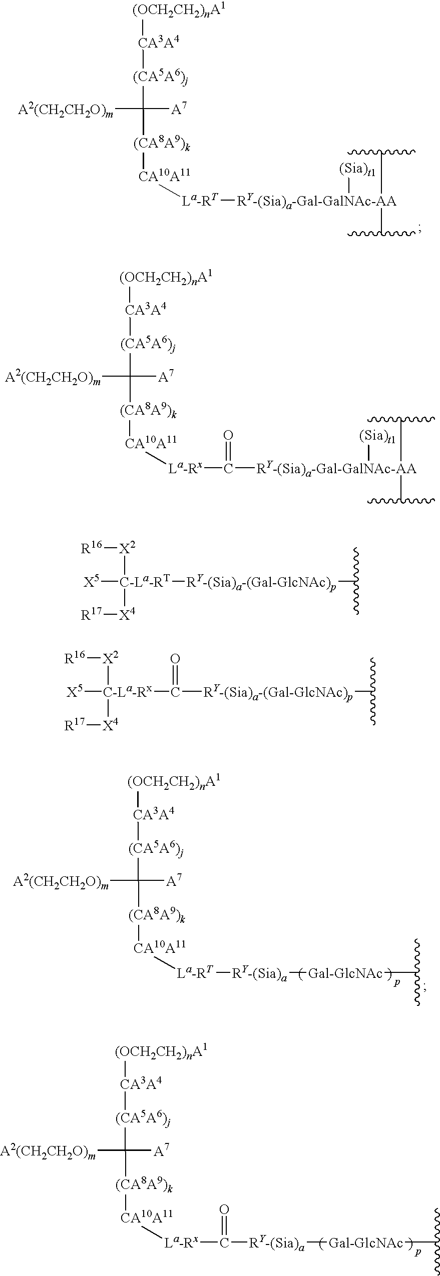

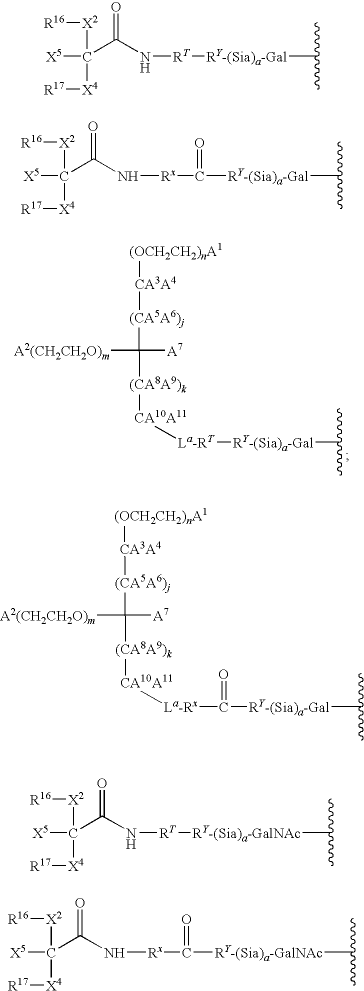

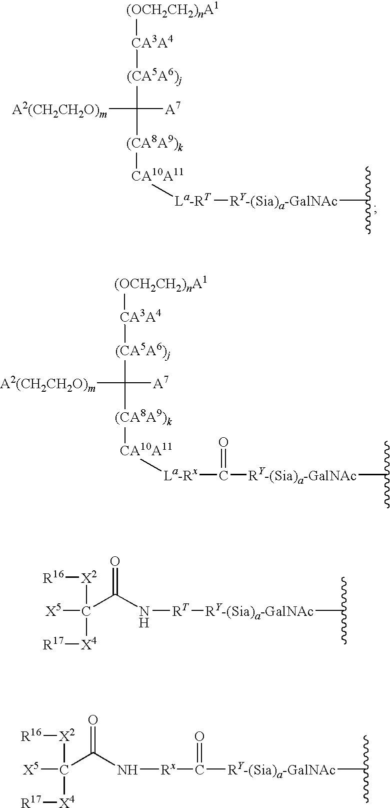

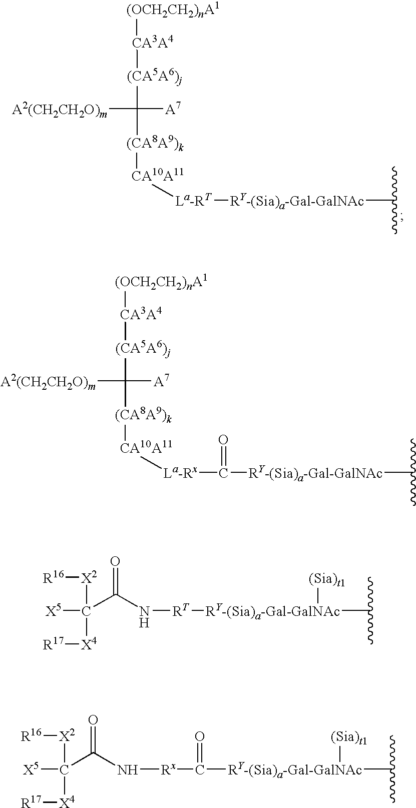

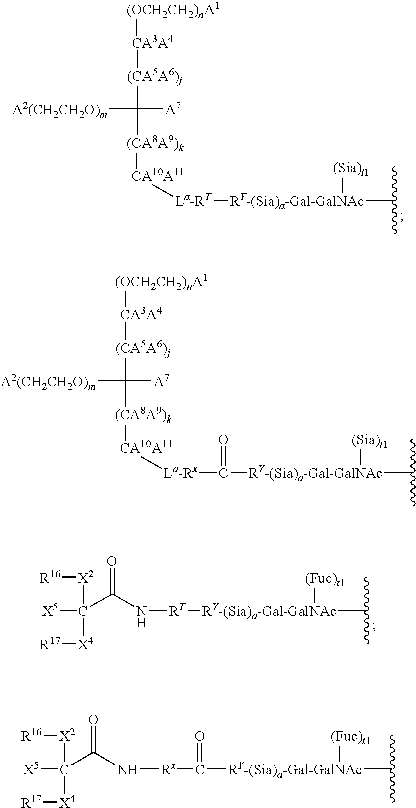

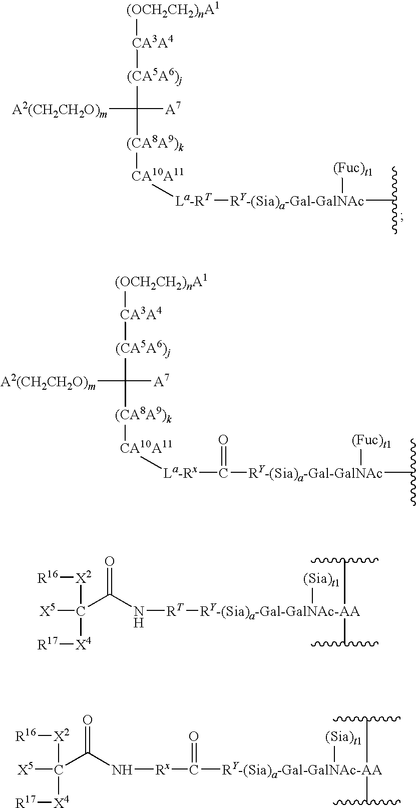

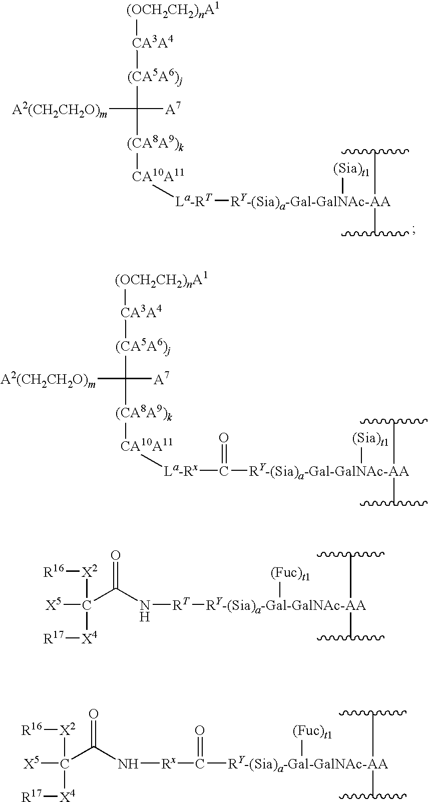

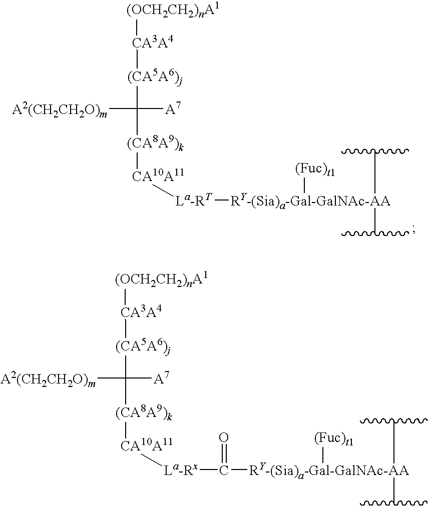

in which the index a and the linker L^{a} are as discussed above. The index p is an integer from 1 to 10. The indices t1 and a are independently selected from 0 or 1. R^{Y} is a member selected from a bond, O, S and NH. Each of these groups can be included as components of the mono-, bi-, tri- and tetra-antennary saccharide structures set forth above. AA is an amino acid residue of the peptide.

In an exemplary embodiment, a lipoPEGylated peptide conjugate of the invention is selected from the formulae set forth below:

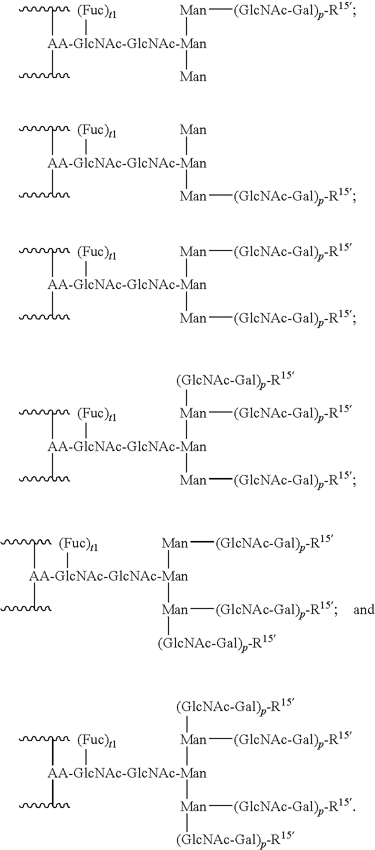

In the formulae above, the index t1 is an integer from 0 to 1 and the index p is an integer from 1 to 10. The symbol R^{15′} represents H, OH (e.g., Sia-OH, Gal-OH), a modified lipid group, a sialyl moiety, or a sialyl moiety covalently attached to a modified lipid group. An exemplary peptide conjugate of the invention will include at least one glycan having a R^{15′} that includes a structure according to Formulae I or II. In an exemplary embodiment, the modified lipid is linked to the galactose residue. In another exemplary embodiment, the modified lipid is linked to the galactose residue.

In another exemplary embodiment, the glycans on the peptide conjugates have a formula that is selected from the group:

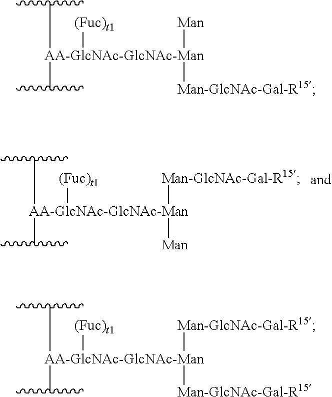

and combinations thereof.

In each of the formulae above, R^{15′} is as discussed above. Moreover, an exemplary peptide conjugate of the invention will include at least one glycan with an R^{15′} moiety that includes a structure according to Formulae I or II.

In another exemplary embodiment, the peptide conjugate comprises at least one structure having the formula:

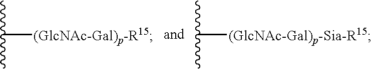

wherein R¹⁵ is a modified lipid; and the index p is an integer selected from 1 to 10.

### IV. d) Modifying Group Linker

The linkers of the modifying group serve to attach the modifying group (ie polymeric modifying groups, targeting moieties, therapeutic moieties and biomolecules) to the lipid linking group. In an exemplary embodiment, the modifying group linker is bound to a lipid linking group, generally through a heteroatom, e.g., nitrogen, as shown below:

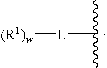

In this diagram, R¹ is the modifying group and L is a member selected from a bond and a modifying group linker. The index w represents an integer selected from 1-6, preferably 1-3 and more preferably 1-2. When w is greater than one, the modifying group—modifying group linker construct is a branched structure that includes two or more modifying groups attached to L.

In another exemplary embodiment, the structure has a formula as shown below:

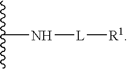

In another exemplary embodiment, the structure has a formula as shown below:

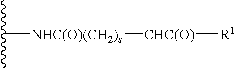

in which s is an integer from 0 to 20 and R¹ is a modifying group.

When L is a bond it is formed between a reactive functional group on a precursor of R¹ and a reactive functional group of complementary reactivity on a precursor of a lipid linking group.

In an exemplary embodiment, L is a modifying group linker that is formed from an amino acid, or small peptide (e.g., 1-4 amino acid residues) providing a modified lipid in which the polymeric modifying group is attached through a substituted alkyl linker. Exemplary modifying group linkers include glycine, lysine, serine and cysteine. The PEG moiety can be attached to the amine moiety of the modifying group linker through an amide or urethane bond. The PEG is linked to the sulfur or oxygen atoms of cysteine and serine through thioether or ether bonds, respectively.

### IV. e) Modifying Group

The modifying groups of the invention can be water-soluble polymers, water-insoluble polymers, therapeutic moieties, diagnostic moieties, targeting moieties and biomolecules.

### IV. e) i) Water-Soluble Polymers

Many water-soluble polymers are known to those of skill in the art and are useful in practicing the present invention. The term water-soluble polymer encompasses species such as saccharides (e.g., dextran, amylose, hyalouronic acid, poly(sialic acid), heparans, heparins, etc.); poly(amino acids), e.g., poly(aspartic acid) and poly(glutamic acid); nucleic acids; synthetic polymers (e.g., poly(acrylic acid), poly(ethers), e.g., poly(ethylene glycol)); peptides, proteins, and the like. The present invention may be practiced with any water-soluble polymer with the sole limitation that the polymer must include a point at which the remainder of the conjugate can be attached.

In an exemplary embodiment, the water-soluble polymer is a poly(peptide), and the poly(peptide) is an enzyme. In another exemplary embodiment, the water-soluble polymer is a poly(saccharide), and the poly(saccharide) is poly(sialic acid). In an exemplary embodiment, the water-soluble polymer is a poly(ether), and the poly(ether) is a poly(ethylene glycol) which is a member selected from linear PEG and branched PEG.

Methods for activation of polymers can also be found in WO 94/17039, U.S. Pat. No. 5,324,844, WO 94/18247, WO 94/04193, U.S. Pat. No. 5,219,564, U.S. Pat. No. 5,122,614, WO 90/13540, U.S. Pat. No. 5,281,698, and more WO 93/15189, and for conjugation between activated polymers and peptides, e.g. Coagulation Factor VIII (WO 94/15625), hemoglobin (WO 94/09027), oxygen carrying molecule (U.S. Pat. No. 4,412,989), ribonuclease and superoxide dismutase (Veronese at al., App. Biochem. Biotech. 11: 141-45 (1985)).

Exemplary water-soluble polymers are those in which a substantial proportion of the polymer molecules in a sample of the polymer are of approximately the same molecular weight; such polymers are “homodisperse.”

The present invention is further illustrated by reference to a poly(ethylene glycol) conjugate. Several reviews and monographs on the functionalization and conjugation of PEG are available. See, for example, Harris, Macronol. Chem. Phys. C25: 325-373 (1985); Scouten, Methods in Enzymology 135: 30-65 (1987); Wong et al., Enzyme Microb. Technol. 14: 866-874 (1992); Delgado et al., Critical Reviews in Therapeutic Drug Carrier Systems 9: 249-304 (1992); Zalipsky, Bioconjugate Chem. 6: 150-165 (1995); and Bhadra, et al., Pharmazie, 57:5-29 (2002). Routes for preparing reactive PEG molecules and forming conjugates using the reactive molecules are known in the art. For example, U.S. Pat. No. 5,672,662 discloses a water soluble and isolatable conjugate of an active ester of a polymer acid selected from linear or branched poly(alkylene oxides), poly(oxyethylated polyols), poly(olefinic alcohols), and poly(acrylomorpholine).

U.S. Pat. No. 6,376,604 sets forth a method for preparing a water-soluble 1-benzotriazolylcarbonate ester of a water-soluble and non-peptidic polymer by reacting a terminal hydroxyl of the polymer with di(1-benzotriazoyl)carbonate in an organic solvent. The active ester is used to form conjugates with a biologically active agent such as a protein or peptide.

WO 99/45964 describes a conjugate comprising a biologically active agent and an activated water soluble polymer comprising a polymer backbone having at least one terminus linked to the polymer backbone through a stable linkage, wherein at least one terminus comprises a branching moiety having proximal reactive groups linked to the branching moiety, in which the biologically active agent is linked to at least one of the proximal reactive groups. Other branched poly(ethylene glycols) are described in WO 96/21469, U.S. Pat. No. 5,932,462 describes a conjugate formed with a branched PEG molecule that includes a branched terminus that includes reactive functional groups. The free reactive groups are available to react with a biologically active species, such as a protein or peptide, forming conjugates between the poly(ethylene glycol) and the biologically active species. U.S. Pat. No. 5,446,090 describes a bifunctional PEG linker and its use in forming conjugates having a peptide at each of the PEG linker termini.

Conjugates that include degradable PEG linkages are described in WO 99/34833; and WO 99/14259, as well as in U.S. Pat. No. 6,348,558. Such degradable linkages are applicable in the present invention.

The art-recognized methods of polymer activation set forth above are of use in the context of the present invention in the formation of the branched polymers set forth herein and also for the conjugation of these branched polymers to other species, e.g., sugars, sugar nucleotides and the like.

An exemplary water-soluble polymer is poly(ethylene glycol), e.g., methoxy-poly(ethylene glycol). The poly(ethylene glycol) used in the present invention is not restricted to any particular form or molecular weight range. For unbranched poly(ethylene glycol) molecules the molecular weight is preferably between 500 and 100,000. A molecular weight of 2,000-60,000 is preferably used and preferably of from about 5,000 to about 40,000.

Prior to conjugation, the poly(ethylene glycol) molecules of the invention include, but are not limited to, those species set forth below.

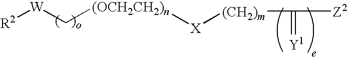

in which R² is H, substituted or unsubstituted alkyl, substituted or unsubstituted aryl, substituted or unsubstituted heteroaryl, substituted or unsubstituted heterocycloalkyl, substituted or unsubstituted heteroalkyl, e.g., acetal, OHC—, H₂N—CH₂CH₂—, HS—CH₂CH₂—, and —(CH₂)_{q}C(Y¹)Z¹; -sugar-nucleotide, or protein. The index “n” represents an integer from 1 to 2500. The indices m and o independently represent integers from 0 to 20. The index q is an integer selected from 1 to 2500. The index e represents an integer from 0 to 1. The symbol Z² represents OH, NH₂, halogen, S—R³, the alcohol portion of activated esters, —(CH₂)ₚC(Y²)V, —(CH₂)ₚU(CH₂)ₙC(Y²)ᵥ sugar-nucleotide, protein, and leaving groups, e.g., imidazole, p-nitrophenyl, HOBT, tetrazole, halide. The symbols X, Y¹, Y², W and U independently represent the moieties O, S, N—R⁴. The symbol V represents OH, NH₂, halogen, S—R⁵, the alcohol component of activated esters, the amine component of activated amides, sugar-nucleotides, and proteins. The indices p, s and v are members independently selected from the integers from 0 to 20. The symbols R³, R⁴ and R⁵ independently represent H, substituted or unsubstituted alkyl, substituted or unsubstituted heteroalkyl, substituted or unsubstituted aryl, substituted or unsubstituted heterocycloalkyl and substituted or unsubstituted heteroaryl. After conjugation to the lipid linking group, Z² is converted to Z¹, which is a member selected from —(CH₂)ₚC(Y²)V, —(CH₂)ₚU(CH₂)ₙC(Y²)ᵥ, O, S and N—R⁴.

In other exemplary embodiments, the poly(ethylene glycol) molecule is selected from the following:

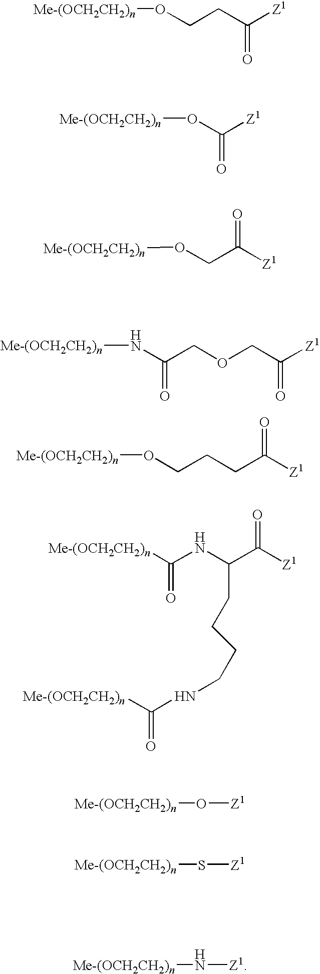

In another exemplary embodiment, the modifying groups of the invention include:

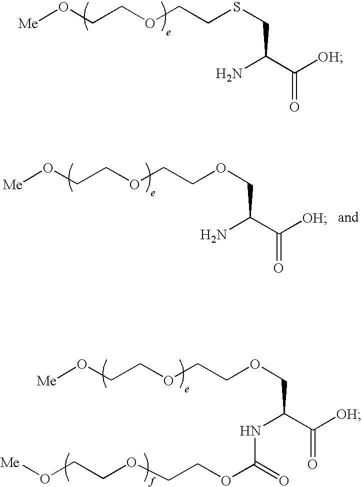

and carbonates and active esters of these species, such as:

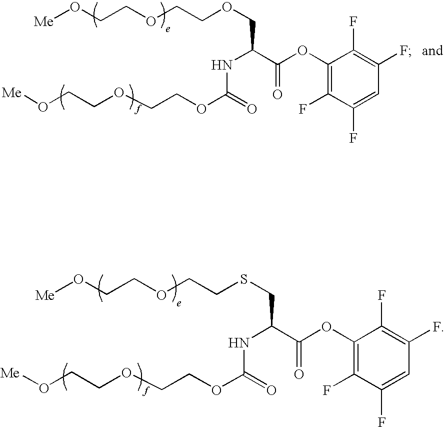

Other activating, or leaving groups, appropriate for activating PEGs of use in preparing the compounds set forth herein include, but are not limited to the species:

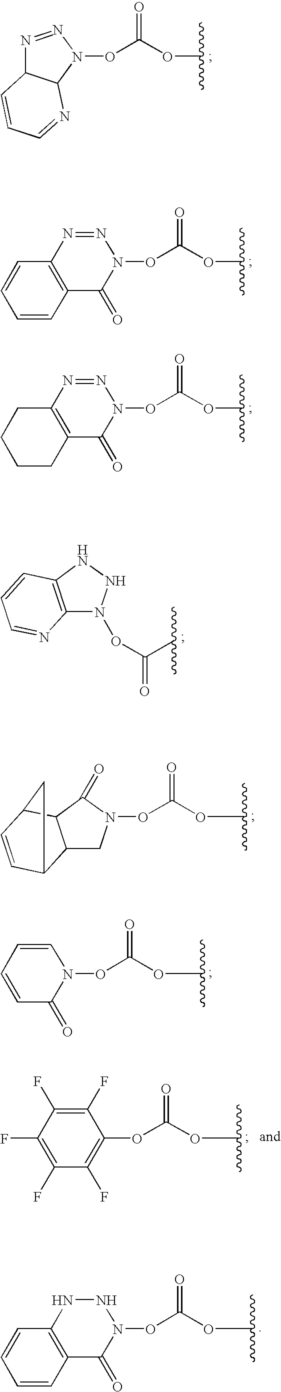

PEG molecules that are activated with these and other species and methods of making the activated PEGs are set forth in WO 04/083259.

Those of skill in the art will appreciate that one or more of the m-PEG arms of the branched polymer can be replaced by a PEG moiety with a different terminus, e.g., OH, COOH, NH₂, C₂-C₁₀-alkyl, etc. Moreover, the structures above are readily modified by inserting alkyl linkers (or removing carbon atoms) between the α-carbon atom and the functional group of the side chain. Thus, “homo” derivatives and higher homologues, as well as lower homologues are within the scope of cores for branched PEGs of use in the present invention.

In another embodiment the poly(ethylene glycol) is a branched PEG having more than one PEG moiety attached. Examples of branched PEGs are described in U.S. Pat. No. 5,932,462; U.S. Pat. No. 5,342,940; U.S. Pat. No. 5,643,575; U.S. Pat. No. 5,919,455; U.S. Pat. No. 6,113,906; U.S. Pat. No. 5,183,660; WO 02/09766; Kodera Y., Bioconjugate Chemistry 5: 283-288 (1994); and Yamasaki et al., Agric. Biol. Chem., 52: 2125-2127, 1998. In a preferred embodiment the molecular weight of each poly(ethylene glycol) of the branched PEG is less than or equal to 40,000 daltons.

Representative branched water-soluble polymers include structures that are based on side chain-containing amino acids, e.g., serine, cysteine, lysine, and small peptides, e.g., lys-lys. Exemplary structures include:

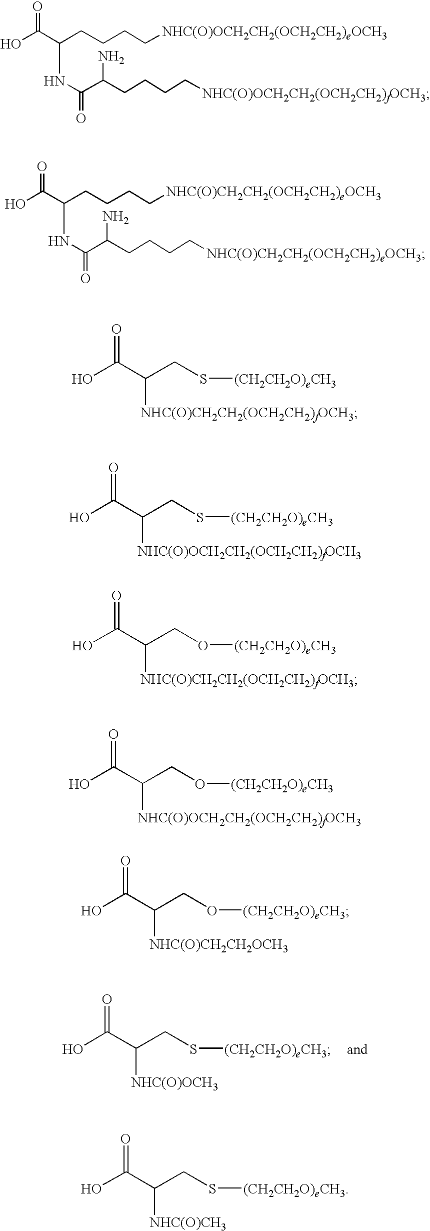

Those of skill will appreciate that the free amine in the di-lysine structures can also be pegylated through an amide or urethane bond with a PEG moiety.

In yet another embodiment, the modifying group is a branched PEG moiety that is based upon a tri-lysine peptide. The tri-lysine can be mono-, di-, tri-, or tetra-PEG-ylated. Exemplary species according to this embodiment have the formulae:

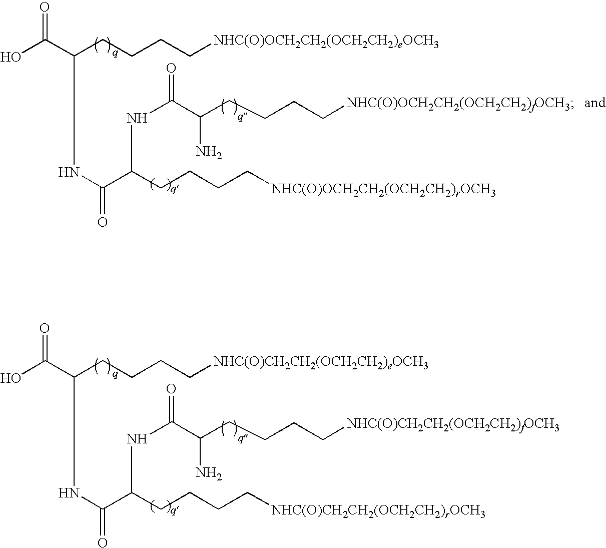

in which the indices e, f and f′ are independently selected integers from 1 to 2500; and the indices q, q′ and q″ are independently selected integers from 1 to 20.

In exemplary embodiments of the invention, the PEG is m-PEG (5 kD, 10 kD, or kD). An exemplary branched PEG species is a serine- or cysteine-(m-PEG)₂ in which the m-PEG is a 20 kD m-PEG.

As will be apparent to those of skill, the branched polymers of use in the invention include variations on the themes set forth above. For example the di-lysine-PEG conjugate shown above can include three polymeric subunits, the third bonded to the α-amine shown as unmodified in the structure above. Similarly, the use of a tri-lysine functionalized with three or four polymeric subunits labeled with the polymeric modifying moiety in a desired manner is within the scope of the invention.

As discussed herein, the PEG of use in the peptide conjugates of the invention can be linear or branched. An exemplary precursor of use to form the branched PEG containing peptide conjugates according to this embodiment of the invention has the formula:

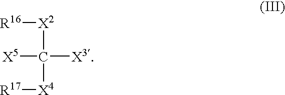

Another exemplary precursor of use to form the branched PEG containing peptide conjugates according to this embodiment of the invention has the formula:

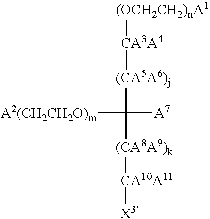

in which the indices m and n are integers independently selected from 0 to 5000. A¹, A², A³, A⁴, A⁵, A⁶, A⁷, A⁸, A⁹, A¹⁰ and A¹¹ are members independently selected from H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —NA¹²A¹³, —OA¹² and —SiA¹²A¹³. A¹² and A¹³ are members independently selected from substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

The branched polymer species according to this formula are essentially pure water-soluble polymers. X^{3′} is a moiety that includes an ionizable (e.g., OH, COOH, H₂PO₄, HSO₃, NH₂, and salts thereof, etc.) or other reactive functional group, e.g., infra. C is carbon. X⁵, R¹⁶ and R¹⁷ are independently selected from non-reactive groups (e.g., H, unsubstituted alkyl, unsubstituted heteroalkyl) and polymeric arms (e.g., PEG). X² and X⁴ are linkage fragments that are preferably essentially non-reactive under physiological conditions, which may be the same or different. An exemplary linker includes neither aromatic nor ester moieties. Alternatively, these linkages can include one or more moiety that is designed to degrade under physiologically relevant conditions, e.g., esters, disulfides, etc. X² and X⁴ join polymeric arms R¹⁶ and R¹⁷ to C. When X^{3′} is reacted with a reactive functional group of complementary reactivity on a modifying group linker or lipid linking group, X^{3′} is converted to a component of linkage fragment X³.

Exemplary linkage fragments for X², X³ and X⁴ are independently selected and include S, SC(O)NH, HNC(O)S, SC(O)O, O, NH, NHC(O), (O)CNH and NHC(O)O, and OC(O)NH, CH₂S, CH₂O, CH₂CH₂O, CH₂CH₂S, (CH₂)₀O, (CH₂)₀S or (CH₂)₀Y′-PEG wherein, Y′ is S, NH, NHC(O), C(O)NH, NHC(O)O, OC(O)NH, or O and o is an integer from 1 to 50. In an exemplary embodiment, the linkage fragments X² and X⁴ are different linkage fragments.

In an exemplary embodiment, the precursor (Formula III), or an activated derivative thereof, is reacted with, and thereby bound to a lipid, an activated lipid through a reaction between X^{3′} and a group of complementary reactivity on the lipid linking group, e.g., an amine. Alternatively, X^{3′} reacts with a reactive functional group on a precursor to modifying group linker, L.

In an exemplary embodiment, the moiety:

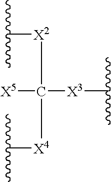

is the modifying group linker, L. In this embodiment, an exemplary linker is derived from a natural or unnatural amino acid, amino acid analogue or amino acid mimetic, or a small peptide formed from one or more such species. For example, certain branched polymers found in the compounds of the invention have the formula:

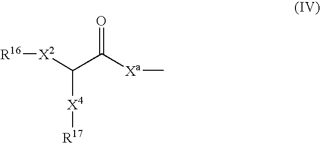

X^{a} is a linkage fragment that is formed by the reaction of a reactive functional group, e.g., X^{3′}, on a precursor of the branched polymeric modifying moiety and a reactive functional group on the sugar moiety, or a precursor to a linker. For example, when X^{3′} is a carboxylic acid, it can be activated and bound directly to an amine group pendent from an amino-saccharide (e.g., Sia, GalNH₂, GlcNH₂, ManNH₂, etc.), forming a X^{a} that is an amide. Additional exemplary reactive functional groups and activated precursors are described hereinbelow. The index c represents an integer from 1 to 10. The other symbols have the same identity as those discussed above.

In another exemplary embodiment, X^{a} is a linking moiety formed with another linker:

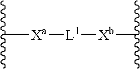

in which X^{b} is a second linkage fragment and is independently selected from those groups set forth for X^{a}, and, similar to L, L¹ is a bond, substituted or unsubstituted alkyl or substituted or unsubstituted heteroalkyl.

Exemplary species for X^{a} and X^{b} include S, SC(O)NH, HNC(O)S, SC(O)O, O, NH, NHC(O), C(O)NH and NHC(O)O, and OC(O)NH.

In another exemplary embodiment, X⁴ is a peptide bond to R¹⁷, which is an amino acid, di-peptide (e.g., Lys-Lys) or tri-peptide (e.g., Lys-Lys-Lys) in which the alpha-amine moiety(ies) and/or side chain heteroatom(s) are modified with a modifying group such as a water-soluble polymer.

In a further exemplary embodiment, the peptide conjugates of the invention include a moiety, e.g., an R¹⁵ or R^{15′} moiety that has a formula that is selected from:

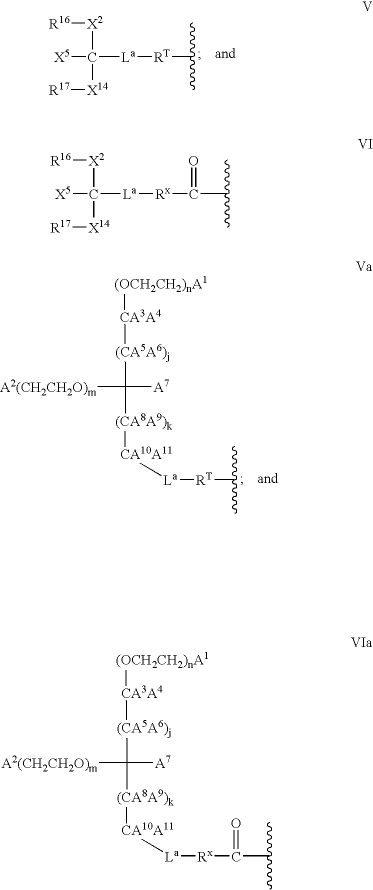

in which the identity of the radicals represented by the various symbols is the same as that discussed hereinabove. L^{a} is a bond or a linker as discussed above for L and L¹, e.g., substituted or unsubstituted alkyl or substituted or unsubstituted heteroalkyl moiety. In an exemplary embodiment, L^{a} is a moiety of the side chain of sialic acid that is functionalized with the modifying group as shown. Exemplary L^{a} moieties include substituted or unsubstituted alkyl chains that include one or more OH or NH₂.

In yet another exemplary embodiment, the invention provides peptide conjugates having a moiety, e.g., an R¹⁵ or R^{15′} moiety with formula:

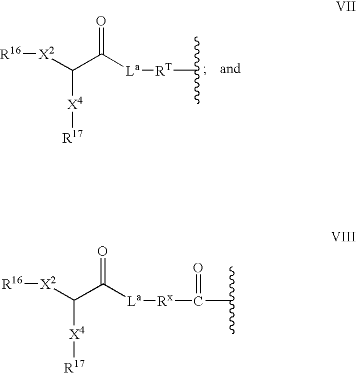

The identity of the radicals represented by the various symbols is the same as that discussed hereinabove. As those of skill will appreciate, the linker arm in Formulae VII and VIII is equally applicable to other modified lipids set forth herein. In exemplary embodiment, the species of Formulae VII and VIII are the R¹⁵ or R^{15′} moieties attached to the structures set forth herein.

In an exemplary embodiment, the lipid linking group has a structure according to the following formula:

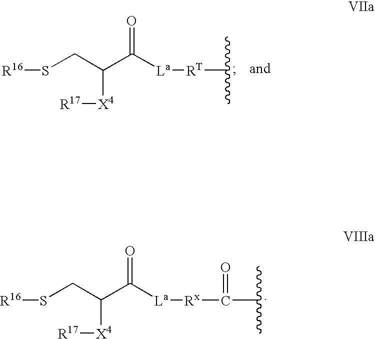

The embodiments of the invention set forth above are further exemplified by reference to species in which the polymer is a water-soluble polymer, particularly poly(ethylene glycol) (“PEG”), e.g., methoxy-poly(ethylene glycol). Those of skill will appreciate that the focus in the sections that follow is for clarity of illustration and the various motifs set forth using PEG as an exemplary polymer are equally applicable to species in which a polymer other than PEG is utilized.

PEG of any molecular weight, e.g., 1 kDa, 2 kDa, 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa and 80 kDa is of use in the present invention.

In an exemplary embodiment, R¹ or L-R¹ or R¹⁵ or R^{15′} is a branched PEG. In an exemplary embodiment, the branched PEG structure is based on a cysteine peptide. Illustrative modified lipids according to this embodiment include those shown below:

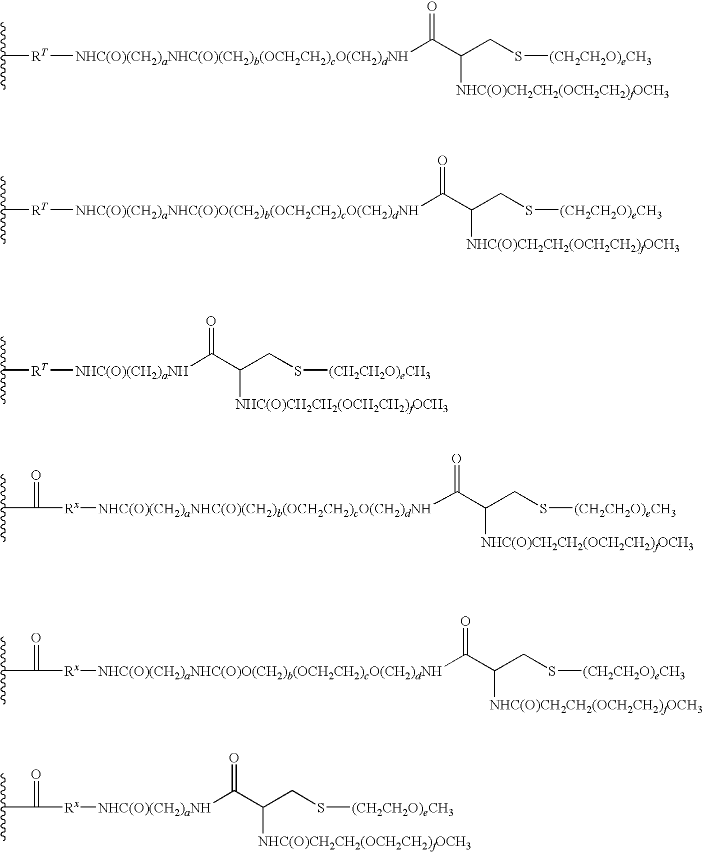

In each of the structures above, the linker fragment —NH(CH₂)ₐ— can be present or absent.

In other exemplary embodiments, the peptide conjugate includes an R¹⁵ or R^{15′} moiety selected from the group:

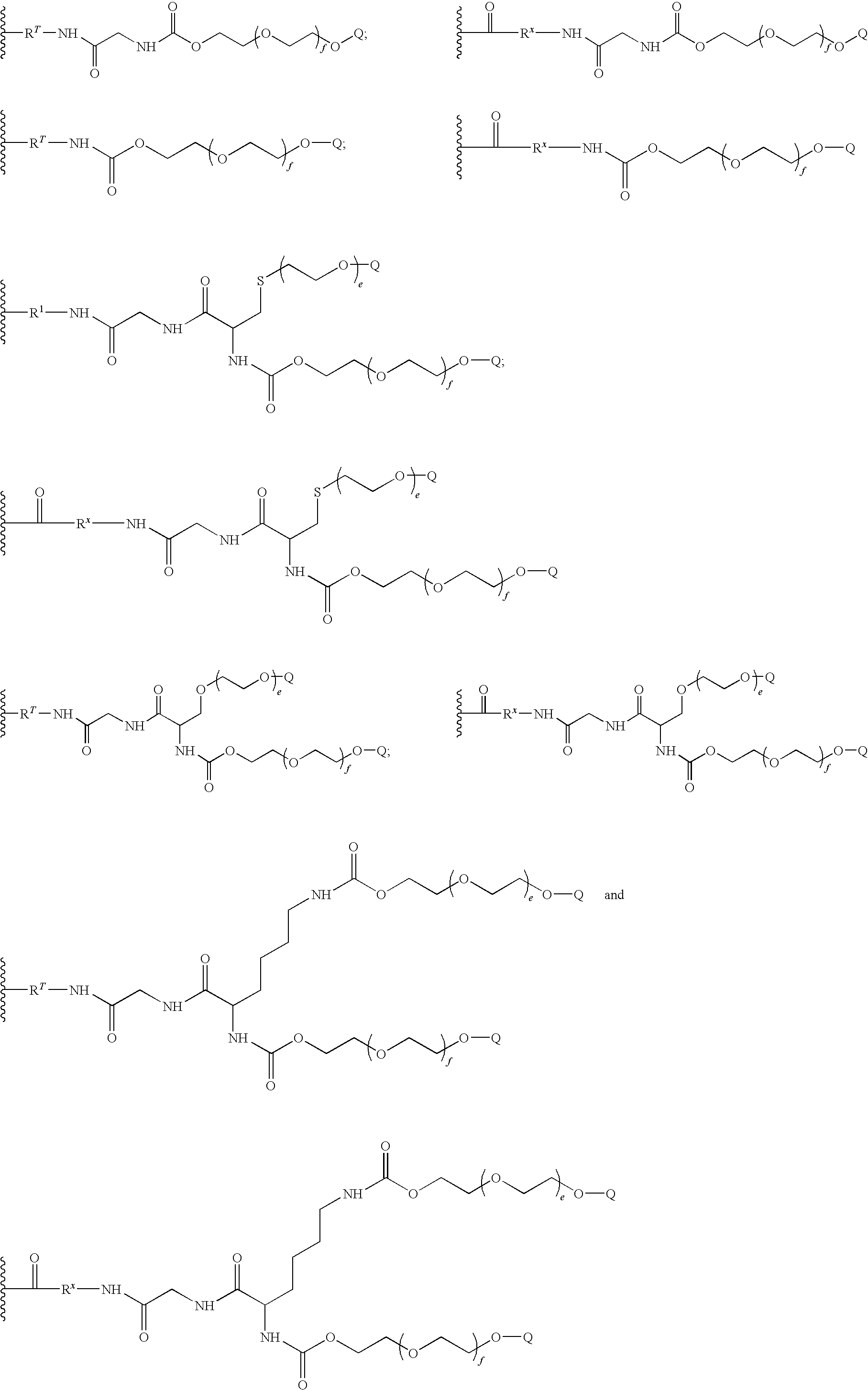

In each of the formulae above, the indices e and f are independently selected from the integers from 1 to 2500. In further exemplary embodiments, e and f are selected to provide a PEG moiety that is about 1 kDa, 2 kDa, 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa and 80 kDa. The symbol Q represents substituted or unsubstituted alkyl (e.g., C₁-C₆ alkyl, e.g., methyl), substituted or unsubstituted heteroalkyl or H.

Other branched polymers have structures based on di-lysine (Lys-Lys) peptides, e.g.:

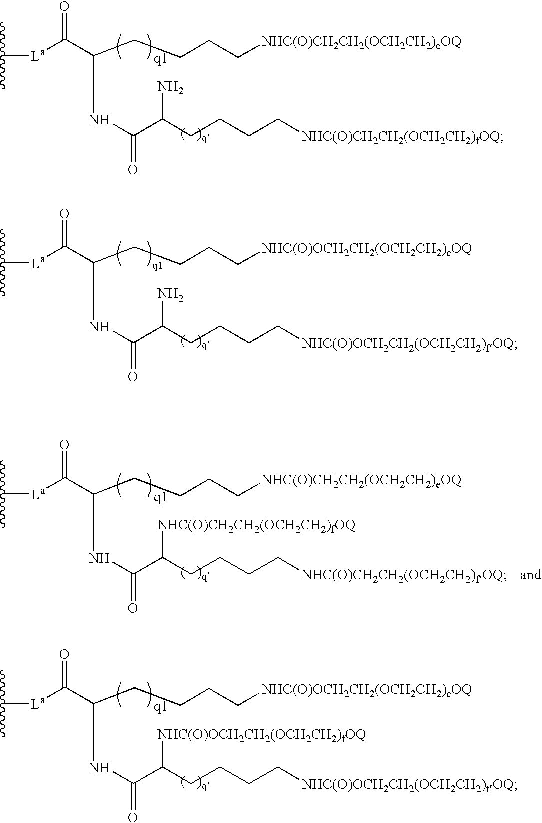

and tri-lysine peptides (Lys-Lys-Lys), e.g.:

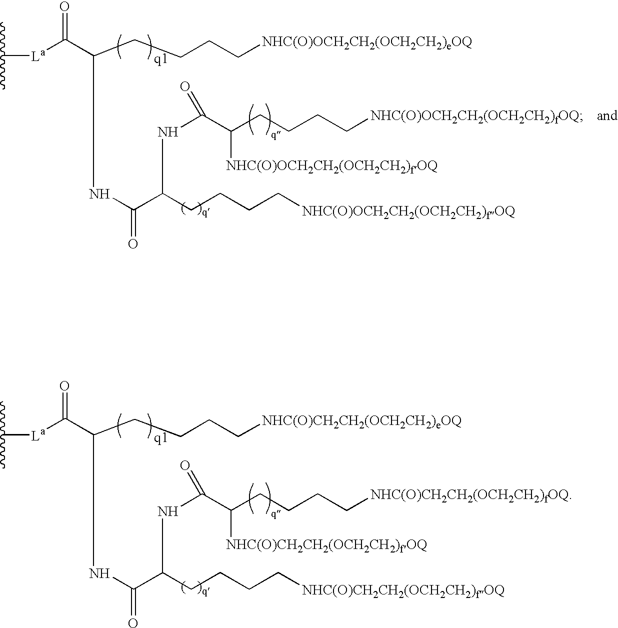

In each of the figures above, the indices e, f, f′ and f″ represent integers independently selected from 1 to 2500. The indices q1, q′ and q″ represent integers independently selected from 1 to 20.

In another exemplary embodiment, the modifying group:

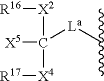

has a formula that is a member selected from:

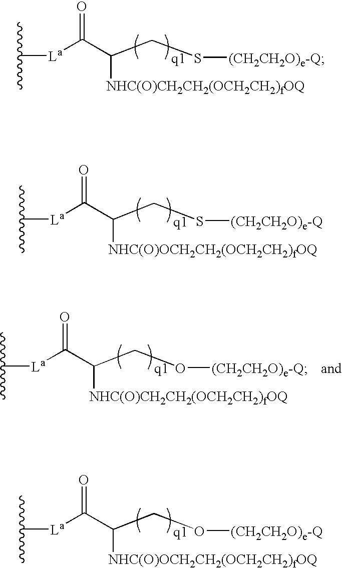

wherein Q is a member selected from H and substituted or unsubstituted C₁-C₆ alkyl. The indices e and f are integers independently selected from 1 to 2500, and the index q1 is an integer selected from 0 to 20.

In another exemplary embodiment, the modifying group:

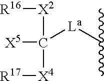

has a formula that is a member selected from:

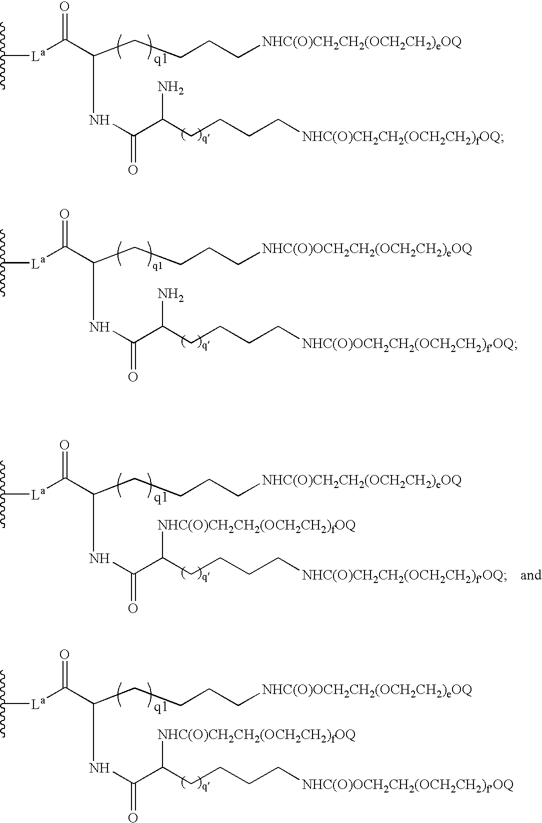

wherein Q is a member selected from H and substituted or unsubstituted C₁-C₆ alkyl. The indices e, f and f′ are integers independently selected from 1 to 2500, and q1 and q′ are integers independently selected from 1 to 20.

In another exemplary embodiment, the branched polymer has a structure according to the following formula:

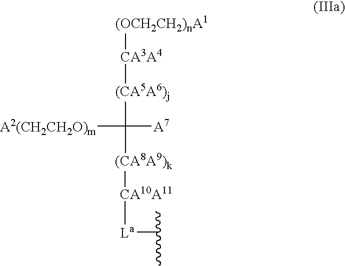

in which the indices m and n are integers independently selected from 0 to 5000. A¹, A², A³, A⁴, A⁵, A⁶, A⁷, A⁸, A⁹, A¹⁰ and A¹¹ are members independently selected from H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —NA¹²A¹³, —OA¹² and —SiA¹²A¹³. A¹² and A¹³ are members independently selected from substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

Formula IIIa is a subset of Formula III. The structures described by Formula IIIa are also encompassed by Formula III.

In another exemplary embodiment according to the formula above, the branched polymer has a structure according to the following formula:

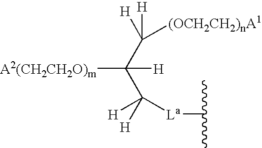

In an exemplary embodiment, A¹ and A² are each —OCH₃ or H.

In an illustrative embodiment, the modified lipids of use in the invention have the formulae:

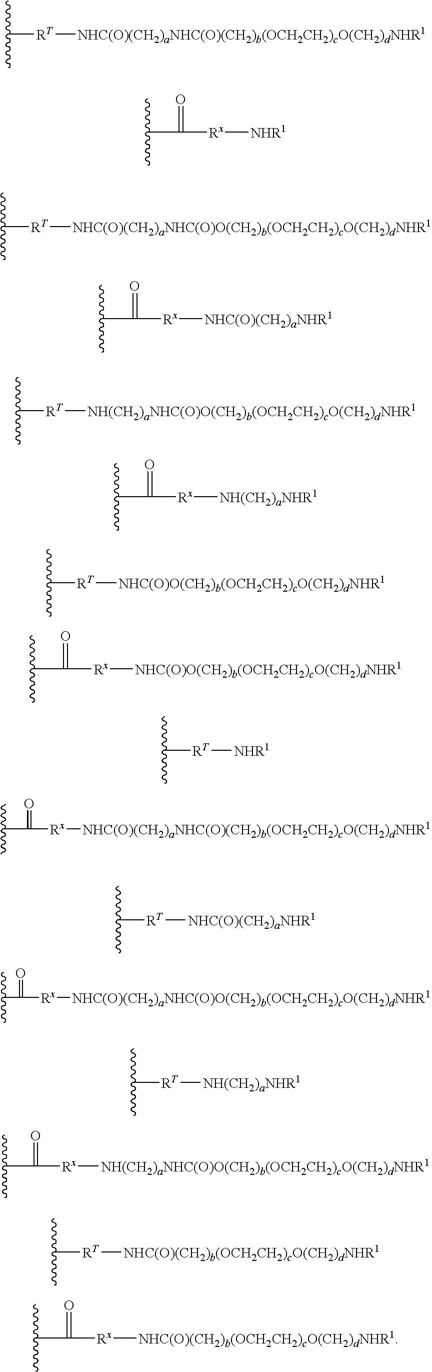

The indices a, b and d are integers from 0 to 20. The index c is an integer from 1 to 2500. The structures set forth above can be components of R¹⁵ or R^{15′}.

Although the present invention is exemplified in the preceding sections by reference to PEG, as those of skill will appreciate, an array of polymeric modifying moieties is of use in the compounds and methods set forth herein.

As discussed herein, the polymer-modified lipids of use in the invention may also be linear structures. Thus, the invention provides for conjugates that include a lipid moiety derived from a structure such as:

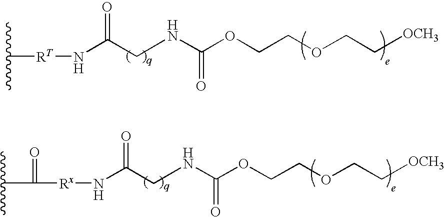

in which the indices q1 and e are as discussed above.

In another exemplary embodiment, the peptide is derived from insect cells, remodeled by adding GlcNAc and Gal to the mannose core and lipopegylated using a lipid bearing a linear PEG moiety, affording a peptide conjugate that comprises at least one moiety having the formula:

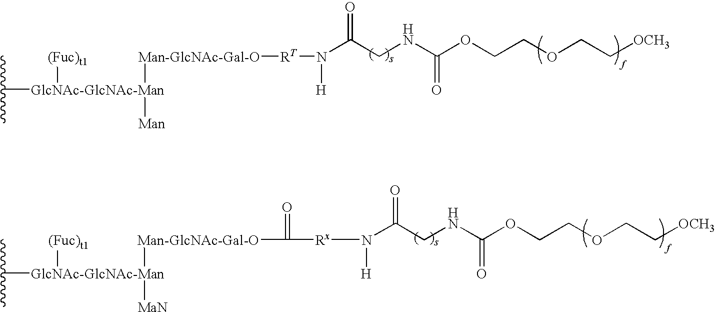

in which the index t1 is an integer from 0 to 1; the index s represents an integer from 1 to 10; and the index f represents an integer from 1 to 2500.

### IV. e) ii) Water-Insoluble Polymers

In another embodiment, analogous to those discussed above, the modified lipids include a water-insoluble polymer, rather than a water-soluble polymer. The conjugates of the invention may also include one or more water-insoluble polymers. This embodiment of the invention is illustrated by the use of the conjugate as a vehicle with which to deliver a therapeutic peptide in a controlled manner. Polymeric drug delivery systems are known in the art. See, for example, Dunn et al., Eds. POLYMERIC DRUGS AND DRUG DELIVERY SYSTEMS, ACS Symposium Series Vol. 469, American Chemical Society, Washington, D.C. 1991. Those of skill in the art will appreciate that substantially any known drug delivery system is applicable to the conjugates of the present invention.

The motifs set forth above for R¹, L-R¹, R¹⁵, R^{15′} and other radicals are equally applicable to water-insoluble polymers, which may be incorporated into the linear and branched structures without limitation utilizing chemistry readily accessible to those of skill in the art. Similarly, the incorporation of these species into any of the modified lipids discussed herein is within the scope of the present invention. Accordingly, the invention provides conjugates containing, and for the use of preparing such conjugates, farnesyl, geranylgeranyl, myristoyl, palmitoyl or other lipid moieties modified with a linear or branched water-insoluble polymers, and activated analogues of the modified lipid species (e.g., (water insoluble polymer)-farnesyl diphosphate or (water insoluble polymer)-palmitoyl-CoA).

Representative water-insoluble polymers include, but are not limited to, polyphosphazines, poly(vinyl alcohols), polyamides, polycarbonates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly (ethylene terephthalate), poly(vinyl acetate), polyvinyl chloride, polystyrene, polyvinyl pyrrolidone, pluronics and polyvinylphenol and copolymers thereof.

Synthetically modified natural polymers of use in conjugates of the invention include, but are not limited to, alkyl celluloses, hydroxyalkyl celluloses, cellulose ethers, cellulose esters, and nitrocelluloses. Particularly preferred members of the broad classes of synthetically modified natural polymers include, but are not limited to, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, cellulose sulfate sodium salt, and polymers of acrylic and methacrylic esters and alginic acid.

These and the other polymers discussed herein can be readily obtained from commercial sources such as Sigma Chemical Co. (St. Louis, Mo.), Polysciences (Warrenton, Pa.), Aldrich (Milwaukee, Wis.), Fluka (Ronkonkoma, N.Y.), and BioRad (Richmond, Calif.), or else synthesized from monomers obtained from these suppliers using standard techniques.

Representative biodegradable polymers of use in the conjugates of the invention include, but are not limited to, polylactides, polyglycolides and copolymers thereof, poly(ethylene terephthalate), poly(butyric acid), poly(valeric acid), poly(lactide-co-caprolactone), poly(lactide-co-glycolide), polyanhydrides, polyorthoesters, blends and copolymers thereof. Of particular use are compositions that form gels, such as those including collagen, pluronics and the like.

The polymers of use in the invention include “hybrid” polymers that include water-insoluble materials having within at least a portion of their structure, a bioresorbable molecule. An example of such a polymer is one that includes a water-insoluble copolymer, which has a bioresorbable region, a hydrophilic region and a plurality of crosslinkable functional groups per polymer chain.

For purposes of the present invention, “water-insoluble materials” includes materials that are substantially insoluble in water or water-containing environments. Thus, although certain regions or segments of the copolymer may be hydrophilic or even water-soluble, the polymer molecule, as a whole, does not to any substantial measure dissolve in water.

For purposes of the present invention, the term “bioresorbable molecule” includes a region that is capable of being metabolized or broken down and resorbed and/or eliminated through normal excretory routes by the body. Such metabolites or break down products are preferably substantially non-toxic to the body.

The bioresorbable region may be either hydrophobic or hydrophilic, so long as the copolymer composition as a whole is not rendered water-soluble. Thus, the bioresorbable region is selected based on the preference that the polymer, as a whole, remains water-insoluble. Accordingly, the relative properties, i.e., the kinds of functional groups contained by, and the relative proportions of the bioresorbable region, and the hydrophilic region are selected to ensure that useful bioresorbable compositions remain water-insoluble.

Exemplary resorbable polymers include, for example, synthetically produced resorbable block copolymers of poly(α-hydroxy-carboxylic acid)/poly(oxyalkylene, (see, Cohn et al., U.S. Pat. No. 4,826,945). These copolymers are not crosslinked and are water-soluble so that the body can excrete the degraded block copolymer compositions. See, Younes et al., J Biomed. Mater. Res. 21: 1301-1316 (1987); and Cohn et al., J Biomed. Mater. Res. 22: 993-1009 (1988).

Presently preferred bioresorbable polymers include one or more components selected from poly(esters), poly(hydroxy acids), poly(lactones), poly(amides), poly(ester-amides), poly(amino acids), poly(anhydrides), poly(orthoesters), poly(carbonates), poly(phosphazines), poly(phosphoesters), poly(thioesters), polysaccharides and mixtures thereof. More preferably still, the bioresorbable polymer includes a poly(hydroxy) acid component. Of the poly(hydroxy) acids, polylactic acid, polyglycolic acid, polycaproic acid, polybutyric acid, polyvaleric acid and copolymers and mixtures thereof are preferred.

In addition to forming fragments that are absorbed in vivo (“bioresorbed”), preferred polymeric coatings for use in the methods of the invention can also form an excretable and/or metabolizable fragment.

Higher order copolymers can also be used in the present invention. For example, Casey et al., U.S. Pat. No. 4,438,253, which issued on Mar. 20, 1984, discloses tri-block copolymers produced from the transesterification of poly(glycolic acid) and an hydroxyl-ended poly(alkylene glycol). Such compositions are disclosed for use as resorbable monofilament sutures. The flexibility of such compositions is controlled by the incorporation of an aromatic orthocarbonate, such as tetra-p-tolyl orthocarbonate into the copolymer structure.

Other polymers based on lactic and/or glycolic acids can also be utilized. For example, Spinu, U.S. Pat. No. 5,202,413, which issued on Apr. 13, 1993, discloses biodegradable multi-block copolymers having sequentially ordered blocks of polylactide and/or polyglycolide produced by ring-opening polymerization of lactide and/or glycolide onto either an oligomeric diol or a diamine residue followed by chain extension with a di-functional compound, such as, a diisocyanate, diacylchloride or dichlorosilane.

Bioresorbable regions of coatings useful in the present invention can be designed to be hydrolytically and/or enzymatically cleavable. For purposes of the present invention, “hydrolytically cleavable” refers to the susceptibility of the copolymer, especially the bioresorbable region, to hydrolysis in water or a water-containing environment. Similarly, “enzymatically cleavable” as used herein refers to the susceptibility of the copolymer, especially the bioresorbable region, to cleavage by endogenous or exogenous enzymes.

When placed within the body, the hydrophilic region can be processed into excretable and/or metabolizable fragments. Thus, the hydrophilic region can include, for example, polyethers, polyalkylene oxides, polyols, poly(vinyl pyrrolidine), poly(vinyl alcohol), poly(alkyl oxazolines), polysaccharides, carbohydrates, peptides, proteins and copolymers and mixtures thereof. Furthermore, the hydrophilic region can also be, for example, a poly(alkylene) oxide. Such poly(alkylene) oxides can include, for example, poly(ethylene) oxide, poly(propylene) oxide and mixtures and copolymers thereof.

Polymers that are components of hydrogels are also useful in the present invention. Hydrogels are polymeric materials that are capable of absorbing relatively large quantities of water. Examples of hydrogel forming compounds include, but are not limited to, polyacrylic acids, sodium carboxymethylcellulose, polyvinyl alcohol, polyvinyl pyrrolidine, gelatin, carrageenan and other polysaccharides, hydroxyethylenemethacrylic acid (HEMA), as well as derivatives thereof, and the like. Hydrogels can be produced that are stable, biodegradable and bioresorbable. Moreover, hydrogel compositions can include subunits that exhibit one or more of these properties.

Bio-compatible hydrogel compositions whose integrity can be controlled through crosslinking are known and are presently preferred for use in the methods of the invention. For example, Hubbell et al., U.S. Pat. Nos. 5,410,016, which issued on Apr. 25, 1995 and 5,529,914, which issued on Jun. 25, 1996, disclose water-soluble systems, which are crosslinked block copolymers having a water-soluble central block segment sandwiched between two hydrolytically labile extensions. Such copolymers are further end-capped with photopolymerizable acrylate functionalities. When crosslinked, these systems become hydrogels. The water soluble central block of such copolymers can include poly(ethylene glycol); whereas, the hydrolytically labile extensions can be a poly(α-hydroxy acid), such as polyglycolic acid or polylactic acid. See, Sawhney et al., Macromolecules 26: 581-587 (1993).

In another preferred embodiment, the gel is a thermoreversible gel. Thermoreversible gels including components, such as pluronics, collagen, gelatin, hyalouronic acid, polysaccharides, polyurethane hydrogel, polyurethane-urea hydrogel and combinations thereof are presently preferred.

In yet another exemplary embodiment, the conjugate of the invention includes a component of a liposome. Liposomes can be prepared according to methods known to those skilled in the art, for example, as described in Eppstein et al., U.S. Pat. No. 4,522,811. For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound or its pharmaceutically acceptable salt is then introduced into the container. The container is then swirled by hand to free lipid material from the sides of the container and to disperse lipid aggregates, thereby forming the liposomal suspension.

The above-recited microparticles and methods of preparing the microparticles are offered by way of example and they are not intended to define the scope of microparticles of use in the present invention. It will be apparent to those of skill in the art that an array of microparticles, fabricated by different methods, is of use in the present invention.

The structural formats discussed above in the context of the water-soluble polymers, both straight-chain and branched are generally applicable with respect to the water-insoluble polymers as well. Thus, for example, the cysteine, serine, dilysine, and trilysine branching cores can be functionalized with two water-insoluble polymer moieties. The methods used to produce these species are generally closely analogous to those used to produce the water-soluble polymers.

IVe) iii) Biomolecules

In another preferred embodiment, the modifying group can be a biomolecule. In still further preferred embodiments, the biomolecule is a functional protein, enzyme, antigen, antibody, peptide, nucleic acid (e.g., single nucleotides or nucleosides, oligonucleotides, polynucleotides and single- and higher-stranded nucleic acids), lectin, receptor or a combination thereof.

Biomolecules useful in practicing the present invention can be derived from any source. The biomolecules can be isolated from natural sources or they can be produced by synthetic methods. Peptides can be natural peptides or mutated peptides. Mutations can be effected by chemical mutagenesis, site-directed mutagenesis or other means of inducing mutations known to those of skill in the art. Peptides useful in practicing the instant invention include, for example, enzymes, antigens, antibodies and receptors. Antibodies can be either polyclonal or monoclonal; either intact or fragments. The peptides are optionally the products of a program of directed evolution.

Both naturally derived and synthetic peptides and nucleic acids are of use in conjunction with the present invention; these molecules can be attached to a lipid linking group or a crosslinking agent by any available reactive group. For example, peptides can be attached through a reactive amine, carboxyl, sulfhydryl, or hydroxyl group. The reactive group can reside at a peptide terminus or at a site internal to the peptide chain. Nucleic acids can be attached through a reactive group on a base (e.g., exocyclic amine) or an available hydroxyl group on a sugar moiety (e.g., 3′- or 5′-hydroxyl). The peptide and nucleic acid chains can be further derivatized at one or more sites to allow for the attachment of appropriate reactive groups onto the chain. See, Chrisey et al. Nucleic Acids Res. 24: 3031-3039 (1996).

In a further preferred embodiment, the biomolecule is selected to direct the peptide modified by the methods of the invention to a specific tissue, thereby enhancing the delivery of the peptide to that tissue relative to the amount of underivatized peptide that is delivered to the tissue. In a still further preferred embodiment, the amount of derivatized peptide delivered to a specific tissue within a selected time period is enhanced by derivatization by at least about 20%, more preferably, at least about 40%, and more preferably still, at least about 100%. Presently, preferred biomolecules for targeting applications include antibodies, hormones and ligands for cell-surface receptors.

In still a further exemplary embodiment, there is provided as conjugate with biotin. Thus, for example, a selectively biotinylated peptide is elaborated by the attachment of an avidin or streptavidin moiety bearing one or more modifying groups.

### V. Methods of Making the Peptide Conjugates

In addition to the compositions discussed above, the present invention provides methods for preparing peptide conjugates including a lipid-based linker and a modifying group. Moreover, the invention provides methods of preventing, curing or ameliorating a disease state by administering a peptide conjugate of the invention to a subject at risk of developing the disease or to a subject who has the disease.

Thus, the invention provides a method of forming a peptide conjugate between a modified lipid and a peptide. For clarity of illustration, the invention is illustrated with reference to a conjugate formed between a peptide and an activated modified lipid group including a lipid and a modifying group, such as a water-soluble polymer. Those of skill will appreciate that the invention equally encompasses methods of forming peptide conjugates with modifying groups other than water-soluble polymers.

In exemplary embodiments, the invention provides a method of producing a peptide conjugate by contacting the peptide with an activated modified lipid comprising a lipid linking group and a water-soluble polymer, and an enzyme for which the activated modified lipid is a substrate. The components of the reaction mixture are combined under conditions appropriate to link the activated modified lipid to a glycosyl residue or an amino acid residue on the peptide, thereby preparing the conjugate.

### V. a) Methods of Making the Lipid Linking Group

In an exemplary embodiment, the peptide conjugates of the invention are produced by contacting a peptide with: (i) a modified lipid precursor having a formula selected from

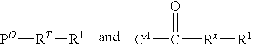

in which P^{O} is a monophosphate or diphosphate, C^{A} is a carboxylic acid activating moiety including, but not limited to, N-hydroxysuccinimidyl, N-hydroxybenztriazolyl, halogen, substituted or unsubstituted imidazolyl, thioethers, p-nitrophenyl ethers, alkyl, alkenyl, alkynyl and aromatic ethers, and derivatives thereof, R^{X} is a member selected from substituted or unsubstituted, saturated or unsaturated C₁-C₄₀ alkyl, R^{T} includes at least one moiety which has a structure according to the formula:

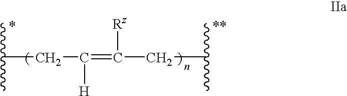

in which R^{z} is a member selected from H and substituted or unsubstituted methyl, and [symbol] and [symbol] describe the points of attachment between said moiety and the remainder of the main chain of the modified lipid. The index n is an integer selected from 1 to 20. R¹ is a modifying group which is a member selected from a water soluble polymer, a water insoluble polymer, a therapeutic moiety and a diagnostic moiety; and (ii) an enzyme for which said modified lipid precursor is a substrate, under conditions appropriate to link the modified lipid precursor to said peptide, thereby preparing the peptide conjugate.

In an exemplary embodiment, the modified lipid precursor is:

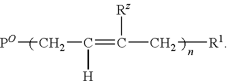

In one embodiment, the lipid linking group is a fatty acid derivative including isoprene moieties. In this embodiment, the peptide conjugate may comprise one or more modified lipids linked through one or more thioester or thioether linkages with cysteine residues of the peptide. In one aspect, modified lipids for use in the invention may be prepared according to one or more of the methods outlined in Scheme 1-3 below.

Scheme 1 sets forth an exemplary route to PEGylated isoprenyl compounds of use in the present invention. Starting compound 1 is produced by protecting a commercially available alcohol (e.g., farnesol, geraniol). The selection of an appropriate protecting agent is within the ability of those of skill in the art.

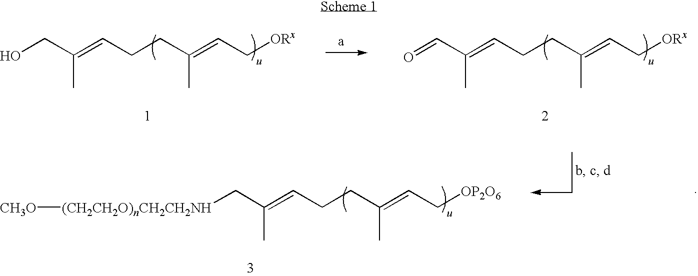

The protected alcohol is then selectively oxidized to compound 1 using an art-recognized method. See, e.g., Bukhtiyarov et al., J. Biol. Chem., 270: 19035-19040 (1995). For example, the alcohol can be formed by the action of t-butyl hydroperoxide and H₂SeO₃.

In step a, the unprotected hydroxyl moiety is selectively oxidized to the corresponding aldehyde. Exemplary oxidation conditions include catalytic oxidation using a supported platinum group metal ion, e.g., Ru—Al—Mg hydrotalcite, Ru—Al—Co hydrotalcite, Pd(II) hydrotalcite, Pd Cluster Complex/TiO₂ and the like. The resulting carbonyl compound, e.g, aldehyde, is reductively aminated with m-PEG-amine (b), and the protecting group is removed (c). The exposed hydroxyl moiety is converted to the corresponding diphosphate (d). See, Holloway et al., Biochem. J., 104: 57-70 (1967). Exemplary phosphorylation conditions for converting the hydroxyl to the diphosphate are bis-(triethylammonium)hydrogen phosphate in the presence of a large excess of CCl₃CN in acetonitrile (Bukhtiyarov et al., supra).

Scheme 2 sets forth a route to compounds of use in a method of the invention in which the m-PEG moiety is tethered to the isoprenyl moiety through an ether linkage. Thus, compound 1 is reacted with an activated m-PEG species, e.g., a halo or sulfonate derivative under conditions appropriate to form the ether (e). The protecting group is removed (c) and the resulting alcohol is phosphorylated as discussed above.

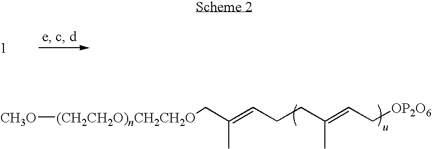

Alternatively, a reactive starting material can be assembled using other recognized methods. See, for example, Mehta et al., The Chemistry of Dienes and Polyenes, Wiley Interscience, NY, 1997.

In another embodiment, a linker is interposed between the m-PEG moiety and the isoprenyl moiety. An exemplary linker is based upon an amino carboxylic acid. Thus, according to Scheme 3, aldehyde 2 is reductively aminated with an amino carboxylic acid (f). The acid is activated, e.g., active ester, acid halide, and coupled with m-PEG amine, forming the corresponding amide (g). The protecting group on the hydroxyl of the amide is removed (c) and the hydroxyl moiety is phosphorylated.

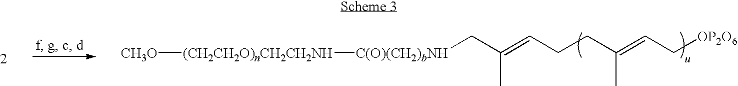

In another embodiment, the lipid linking group is a farnesyl group, and the farnesyl group is enzymatically synthesized by farensyl diphosphate synthetase as disclosed in Szkopinska et al., Acta Biochimica Polonica, 52(1):45-44 (2005).

In another embodiment, the lipid linking group is a fatty acid derivative including a saturated or unsaturated hydrocarbon chain and an acyl moiety. In this embodiment, the fatty acid derivative may be linked to the peptide by a thioester bond with cysteine (i.e. thio-palmitoylation) or in amide linkage to an N-terminal glycine (N-acylation; Knoll et al. Methods in Enzymol. 250:405 (1995)) or an ε-amine of an internal lysine (Hackett M. et al. Science 266:433-435 (1994)). In a further related embodiment, the peptide conjugate may comprise one or more modified lipids including saturated or unsaturated hydrocarbon chains and acyl moieties, and the modified lipids may be independently linked through amide, ester and/or thioester linkages on the same peptide. In one aspect, modified lipids for use in these embodiments of the invention may be prepared according to one or more of the methods outlined in Scheme 4.

Derivatives of palmitic acid can be activated for use with a transferase by converting the carboxylic group to a thioester. In an exemplary embodiment set forth in scheme 4, the thioester is a CoA thioester. In Scheme 4, 16-OH palmitic acid is reacted with an activated poly(ethylene glycol) species under conditions appropriate for the formation of the corresponding ether. The carboxylic acid of the resulting PEG-palmitic acid ether is activated by conversion to an activated ester (e.g., NHS), an anhydride or the like. The activated species is converted to the corresponding Coenzyme A thioester by combining the activated species and Coenzyme A under conditions appropriate for the coupling to occur. The formation of CoA thioesters by this route and other analogous routes is known in the art. See, for example, Kutner et al., Proc. Natl. Acad. Sci. USA. 83: 6781-4 (1986).

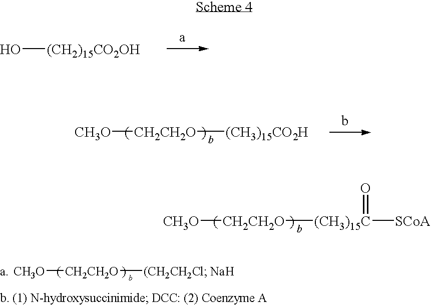

### V. b) Methods of Making the Peptide

The acceptor peptide is typically synthesized de novo, or recombinantly expressed in a prokaryotic cell (e.g., bacterial cell, such as E. coli) or in a eukaryotic cell such as a mammalian, yeast, insect, fungal or plant cell. The peptide can be either a full-length protein or a fragment. Moreover, the peptide can be a wild type or mutated peptide.

### V. c) Methods of Making the Modifying Groups

The branched PEG species set forth herein are readily prepared by methods such as that set forth in the scheme below:

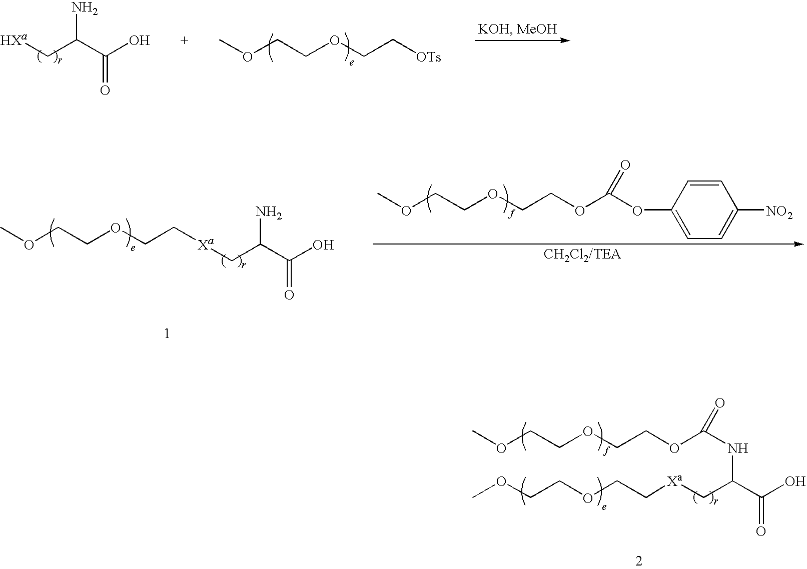

in which X^{a} is O or S and r is an integer from 1 to 5. The indices e and f are independently selected integers from 1 to 2500.

Thus, according to this scheme, a natural or unnatural amino acid is contacted with an activated m-PEG derivative, in this case the tosylate, forming 1 by alkylating the side-chain heteroatom X^{a}. The mono-functionalized m-PEG amino acid is submitted to N-acylation conditions with a reactive m-PEG derivative, thereby assembling branched m-PEG 2. As one of skill will appreciate, the tosylate leaving group can be replaced with any suitable leaving group, e.g., halogen, mesylate, triflate, etc. Similarly, the reactive carbonate utilized to acylate the amine can be replaced with an active ester, e.g., N-hydroxysuccinimide, etc., or the acid can be activated in situ using a dehydrating agent such as dicyclohexylcarbodiimide, carbonyldiimidazole, etc.

### V d) Enzyme Classes

Aspects of the present invention make use of enzymes. Enzymes useful in the practice of the invention include but are not limited to, wild-type and mutant proteases, lipases, esterases, acylases, acyltransferases, glycosyltransferases, sulfotransferases, glycosidases, and the like. In some exemplary embodiments, the enzymes may be wild-type or mutant prenyltransferases (e.g., farnesyltransferases, and geranylgeranyl transferases); N-myristoyltransferases, or palmitoyltransferases.

### V. d) i) Lipid Transfer

In some embodiments, a modifying group is linked to a peptide via a lipid linking arm. In some of these embodiments the lipid linking group is a long chain fatty acid derivative such as palmitate or myristate. In these embodiments, the lipid may be thioesterified to a cysteine residue (i.e. thio-palmitoylation) in varying positions along the polypeptide. Alternatively, the lipid may form an amide linkage to a lysine residue. In those embodiments wherein the lipid linker is a shorter chain fatty acid (e.g. myristate) the lipid is typically in amide linkage to N-terminal glycine (N-acylation).

In some embodiments, the lipid linker is a fatty acid derivative including repeating isoprene units. In these embodiments, an exemplary linkage is the attachment of the modified lipid to the side chain of a cysteine residue through a thioester or a thioether bond. One or more of these thioester or thioether bonds may occur within any given peptide conjugate. In one embodiment, the modified lipid comprises three repeating isoprene units, and the cysteine residue on the peptide is part of an amino acid sequence which comprises CAAX wherein C is cysteine, A is any aliphatic amino acid and X is methionine, glutamine, serine or lysine. In another related embodiment, the modified lipid comprises between 1 to 6 repeating isoprene units, and the cysteine participating in the thioester bond is embedded within the sequence cysteine-cysteine, or cysteine-X-cysteine. In this embodiment, X is any amino acid and the thioester or thioether bond occurs together with another thioester or thioether bond.

In another exemplary linkage the modified lipid is attached to the peptide via an amide bond. In this embodiment, the modified lipid may be attached to the peptide through an amide bond formed with the α-amino group of an N-terminal glycine residue. In other embodiments, the amide linkage may occur with the ε-amino group of an internal lysine residue.

In other embodiments the modified lipid is attached to the peptide through an amide bond formed with the terminal amino group of phosphoethanolamine that comprises a glycophosphatidylinositol anchor. Glycophosphatidylinositol anchors are known in the art. Glycophosphatidylinositol anchors are linked to an amino acid bearing a small side chain (e.g., glycine) at the carboxy-terminal end of membrane proteins which is embedded within a sequence context comprising another, independently selected, small side chain amino acid located two positions further toward the carboxy-terminal end of the protein. The small side chain amino acid two positions carboxy-terminal to the linked amino acid is followed in sequence by 5-10 hydrophilic amino acids, and then by 5-10 hydrophobic amino acids at or near the carboxy terminus (see e.g., Essentials of Glycobiology, Varki, A. et al. eds. CSHL Press (1999)).

Thus, exemplary attachment points for selected modified lipids include, but are not limited to: (a) consensus sites for prenylation, palmitoylation and myristoylation; (b) terminal glycine residues that are acceptors for a myristoyltransferase; (c) acceptor sites for GPI modification; and (d) glycosyl residues which are substrates for the action of lipases/esterases/acyltransferases.

### V. d) ii) Lipases/Esterases/Acyltransferases

Lipases (triacylglycerol lipases EC 3.1.1.3) are enzymes which have been classically employed to carry on hydrolysis of triglycerides with concommitant production of free fatty acids. However these enzymes also display catalytic activity towards a large variety of alcohols and acids in ester synthesis reactions. Exemplary lipases for use in this invention can be found in on-line databases such as the Lipase Engineering Database (www.led.uni-stuttgart.de) and the Lipase Database (www.au-kbc.org/beta/bioproj2/). In an exemplary embodiment, lipases are used to catalyze the attachment of a modified lipid onto a glycosyl residue. These enzymatic attachments can be achieved by lipases from Thermomyces lanuginosus, Candida antarctica, Pseudomonas sp. and Penicillium chrysogenum. Plou et al., J. Biotech., 96:55-66 (2002). In another exemplary embodiment, the enzyme is an acyltransferase involved in the biosynthesis of lipooligosaccharide (LOS) as described in Gilbert and Wakarchuk, U.S. Pat. Pub. No. 20040229313. In another exemplary embodiment, the enzyme is an acetyltransferase such as those described in Satake and Varki, J. Bio. Chem., 278(10):7942-7948 (2003).

V. d) iii) Prenyltransferases

In an exemplary embodiment, the enzyme that transfers a modified lipid group is a prenyltransferase. Protein geranylgeranyltransferase type I (EC 2.5.1.59), along with protein farnesyltransferase (EC 2.5.1.58) and protein geranylgeranyltransferase type II (EC 2.5.1.60), comprise the protein prenyltransferase family of enzymes. Protein geranylgeranyltransferase type I catalyses the formation of a thioether linkage between the C-1 atom of the geranylgeranyl group and a cysteine residue fourth from the C-terminus of the protein. The protein acceptors bearing a C-terminal sequence CA¹A²X, where the terminal residue, X, is preferably leucine; serine, methionine, alanine or glutamine make the protein a substrate for farnesyltransferase (EC 2.5.1.58). The enzymes are relaxed in specificity for A¹, but cannot act if A² is aromatic. Known targets of this enzyme include most g-subunits of heterotrimeric G proteins and Ras-related GTPases such as members of the Ras and Rac/Rho families. Protein geranylgeranyltransferase I is a zinc metalloenzyme. Although the Zn²⁺ is required for peptide binding by the wild-type enzyme, it not required for isoprenoid binding.

As is known in the art, all protein prenyltransferases share a common reaction mechanism. Indeed, J S. Taylor et al. (EMBO J. 2003 November; 22 (22): 5963-5974) converted farnesyltransferase (15-C prenyl substrate) into geranylgeranyltransferase I (20-C prenyl substrate) with a single point mutation.

Geranylgeranyltransferase I typically catalyzes C-terminal lipidation of >100 proteins, including many GTP-binding regulatory proteins. Structural determinants for the posttranslational modification of peptides with isoprenoids are located in the C-terminus of the protein. Indeed, among prenyl acceptors, peptides and proteins with leucine or phenylalanine at their C termini are preferred as geranylgeranyl acceptors, whereas those with C-terminal serine were preferentially farnesylated. Thus, the C-terminal amino acid is an important structural determinant in controlling the specificity of protein prenylation.

### V. d) iv) Myristoyltransferases

In other exemplary embodiments, the modified lipid is transferred by a myristoyltransferase. N-myristoyltransferase (Nmt) is a member of the GCN5-related N-acetyltransferases (GNAT) superfamily of proteins (Dyda, F., et al. (2000) Annu. Rev. Biophys. Biomol. Struct. 29, 81-103). The enzyme catalyses N-myristoylation through an ordered Bi—Bi reaction mechanism, binding first to myristoyl-CoA, with the resulting conformational changes generating a peptide-binding site (Rudnick, et al. (1991) J. Biol. Chem. 266, 9732-9739). Subsequent formation of a ternary myristoyl-CoA:NMT-peptide complex leads to catalysis and product release. Catalysis occurs through a direct nucleophilic addition-elimination reaction.

Nmt can be distinguished from other GNAT family members by the remarkable diversity of its peptide substrates. Known myristoylated proteins include, but are not limited to cAMP-dependent serine/threonine kinases, members of the p60 Src family of tyrosine kinases, retroviral gag polyprotein precursors such as HIV-1pr55, viral capsid components, and the β-subunit of many signal-transducing, heteromeric G proteins. Although some myristoylated proteins are cytosolic, many are associated with cellular membranes where myristoylation facilitates membrane attachment. The addition of myristate is also known in the art to stabilize protein-protein interactions, and many acylated proteins require this modification for full expression of their biological function (see, e.g., McIlhinney, R. A. (1998) Methods Mol. Biol. 88, 211-225).

Typically, though not always, N-myristoylation is an irreversible protein modification that occurs co-translationally following removal of the initiator methionine residue by cellular methionylaminopeptidases (see, e.g., da Silva, A. M., and Klein, C. (1990) J. Cell Biol. 111, 401-407; Wolven, A., et al. (1997) Mol. Biol. Cell 8, 1159-1173 and Towler, D. A., et al. (1987) Proc. Natl. Acad. Sci. U.S.A. 84, 2708-2712). However, N-myristoylation may also occur post-translationally, as in the case of the pro-apoptotic protein BID where proteolytic cleavage by caspase 8 reveals a “hidden” myristoylation motif (Zha, J., et al. (2000) Science 290, 1761-1765). Myristoylation most commonly occurs on an N-terminal glycine, though internal myristoylation of internal glycines, lysines and cysteines is also known (Maurer-Stroh et al., J. Mol. Biol., 317, 523-540 (2002)). Three motif regions over a space of approximately seventeen amino acid residues have been identified by substrate protein sequence analysis as necessary for N-terminal (glycine) myristoylation. Region 1 (residues 1-6) fits the binding pocket of the Nmt; region 2 (residues 7-10), interact with the Nmt's surface at the mouth of the catalytic cavity and region 3 (positions 11-17) includes a hydrophilic, unstructured linker. Further information on the specific amino acid requirements are included in Maurer-Stroh et al., J. Mol. Biol., 317, 523-540 (2002).

### V. d) v) Palmitoyltransferases

In still other exemplary embodiments, the enzyme transferring the modified lipid is a palmitoyltransferase. Palmitoylation involves the addition of palmitate (C16:0) to a peptide. S-palmitoylation refers to the addition of palmitate to cysteine residues through thioester linkages. S-acylation and thioacylation are more general terms used to describe the addition of saturated, monounsaturated and polyunsaturated species of various chain lengths to peptides. Palmitoylation typically occurs post-translationally and is readily reversible. S-palmitoylation may effectively increase the hydrophobicity of proteins or protein domains and thus may contribute to membrane association, subcellular trafficking of proteins between membrane organelles, and trafficking within membrane microdomains. In some cases,

palmitoylation contributes directly in protein-protein interactions. Other palmitoylation motifs are possible, such as oxyester attachement of palmitate or other fatty acids to serine or threonine. Smotrys et al., Annu. Rev. Biochem., 73:559-587 (2004). Amide-linked palmitoylation also occurs. N-palmitoylated proteins include Hedgehog (Hh) proteins which are palmitoylated at the N-terminal cysteine residue and the bacterial Bordatella pertussis adenylate cyclase which is modified with amide-linked palmitate at an internal lysine residue.

A number of proteins are known to be palmitoylated. These proteins include, but are not limited to viral glycoproteins (Schmidt, M. F. G., and Burns, G. R. (1989) Biochem. Soc. Trans. 17, 625-626), p 21 (Guitierrez, L., and Magee, A. I. (1991) Biochim. Biophys. Acta 1078, 147-154), and p 59 (Berthiaume, L., and Resh, M. (1995) J. Biol. Chem. 270, 22399-22405) which is also N-myristoylated.

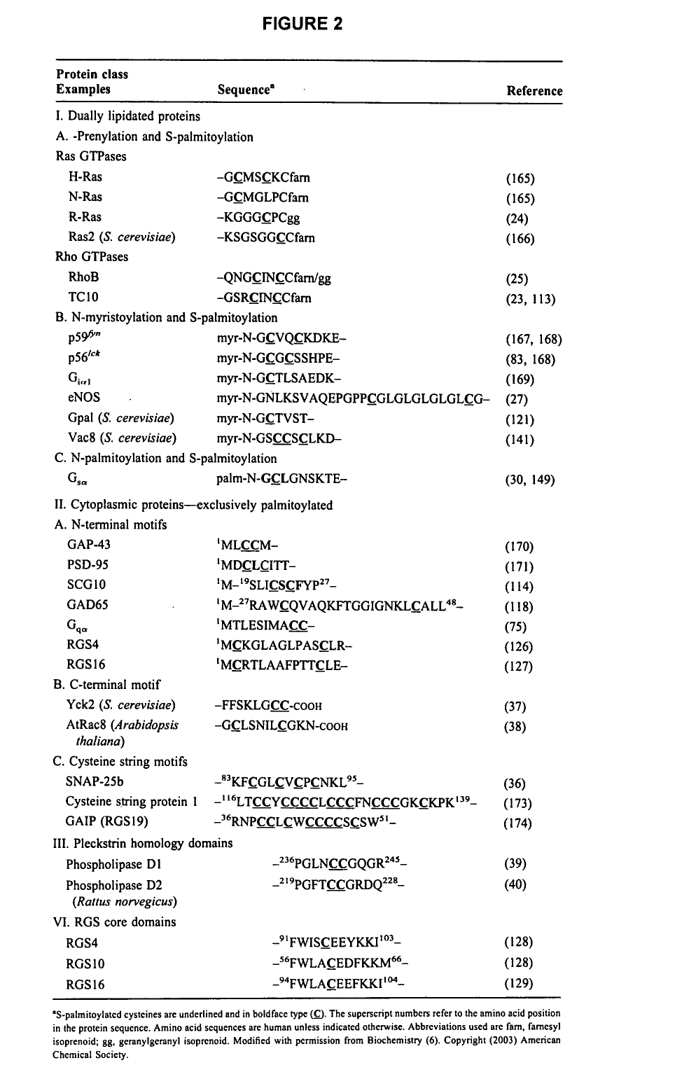

There is no well-defined consensus sequence for palmitoylation; however, a listing of these various sequence motifs is provided in FIG. 2.

### V. d) vi) Sugar Transfer

In addition to the enzymes discussed above in the context of forming the acyl-linked conjugate, the glycosylation pattern of the conjugate and the starting substrates (e.g., peptides, lipids) can be elaborated, trimmed back or otherwise modified by methods utilizing other enzymes. The methods of remodeling peptides and lipids using enzymes that transfer a sugar donor to an acceptor are discussed in great detail in DeFrees, WO 03/031464 A2, published Apr. 17, 2003. A brief summary of selected enzymes of use in the present method is set forth below.

V. d) vii) Glycosyltransferases

Glycosyltransferases catalyze the addition of activated sugars (donor NDP-sugars), in a step-wise fashion, to a protein, glycopeptide, lipid or glycolipid or to the non-reducing end of a growing oligosaccharide. N-linked glycopeptides are synthesized via a transferase and a lipid-linked oligosaccharide donor Dol-PP-NAG₂Glc₃Man₉ in an en block transfer followed by trimming of the core. In this case the nature of the “core” saccharide is somewhat different from subsequent attachments. A very large number of glycosyltransferases are known in the art.

The glycosyltransferase to be used in the present invention may be any as long as it can utilize the modified sugar as a sugar donor. Examples of such enzymes include Leloir pathway glycosyltransferase, such as galactosyltransferase, N-acetylglucosaminyltransferase, N-acetylgalactosaminyltransferase, fucosyltransferase, sialyltransferase, mannosyltransferase, xylosyltransferase, glucurononyltransferase and the like.

For enzymatic saccharide syntheses that involve glycosyltransferase reactions, glycosyltransferase can be cloned, or isolated from any source. Many cloned glycosyltransferases are known, as are their polynucleotide sequences. See, e.g., “The WWW Guide To Cloned Glycosyltransferases,” (http://www.vei.co.uk/TGN/gt_guide.htm). Glycosyltransferase amino acid sequences and nucleotide sequences encoding glycosyltransferases from which the amino acid sequences can be deduced are also found in various publicly available databases, including GenBank, Swiss-Prot, EMBL, and others.

Glycosyltransferases that can be employed in the methods of the invention include, but are not limited to, galactosyltransferases, fucosyltransferases, glucosyltransferases, N-acetylgalactosaminyltransferases, N-acetylglucosaminyltransferases, glucuronyltransferases, sialyltransferases, mannosyltransferases, glucuronic acid transferases, galacturonic acid transferases, and oligosaccharyltransferases. Suitable glycosyltransferases include those obtained from eukaryotes, as well as from prokaryotes.

V. d) viii) Sulfotransferases

The invention also provides methods for producing peptides that include sulfated molecules, including, for example sulfated polysaccharides such as heparin, heparan sulfate, carragenen, and related compounds. Suitable sulfotransferases include, for example, chondroitin-6-sulphotransferase (chicken cDNA described by Fukuta et al., J. Biol. Chem. 270: 18575-18580 (1995); GenBank Accession No. D49915), glycosaminoglycan N-acetylglucosamine N-deacetylase/N-sulphotransferase 1 (Dixon et al., Genomics 26: 239-241 (1995); UL18918), and glycosaminoglycan N-acetylglucosamine N-deacetylase/N-sulphotransferase 2 (murine cDNA described in Orellana et al., J. Biol. Chem. 269: 2270-2276 (1994) and Eriksson et al., J. Biol. Chem. 269: 10438-10443 (1994); human cDNA described in GenBank Accession No. U2304).

### V. d) ix) Glycosidases

This invention also encompasses the use of wild-type and mutant glycosidases. Mutant β-galactosidase enzymes have been demonstrated to catalyze the formation of disaccharides through the coupling of an α-glycosyl fluoride to a galactosyl acceptor molecule. (Withers, U.S. Pat. No. 6,284,494; issued Sep. 4, 2001). Other glycosidases of use in this invention include, for example, β-glucosidases, β-galactosidases, β-mannosidases, β-acetyl glucosaminidases, β-N-acetyl galactosaminidases, β-xylosidases, β-fucosidases, cellulases, xylanases, galactanases, mannanases, hemicellulases, amylases, glucoamylases, α-glucosidases, α-galactosidases, α-mannosidases, α-N-acetyl glucosaminidases, α-N-acetyl galactose-aminidases, α-xylosidases, α-fucosidases, and neuraminidases/sialidases.

### V. d) x) Immobilized Enzymes

The present invention also provides for the use of enzymes that are immobilized on a solid and/or soluble support. In an exemplary embodiment, there is provided an enzyme that is conjugated to a PEG via a lipid linker according to the methods of the invention. The PEG-linker-enzyme conjugate is optionally attached to solid support. The use of solid supported enzymes in the methods of the invention simplifies the work up of the reaction mixture and purification of the reaction product, and also enables the facile recovery of the enzyme. The conjugate is utilized in the methods of the invention. Other combinations of enzymes and supports will be apparent to those of skill in the art.

### V. d) xi) Enzyme Production

### Acquisition of Enzyme Coding Sequences

### General Recombinant Technology

This invention relies on routine techniques in the field of recombinant genetics. Basic texts disclosing the general methods of use in this invention include Sambrook and Russell, Molecular Cloning, A Laboratory Manual (3rd ed. 2001); Kriegler, Gene Transfer and Expression: A Laboratory Manual (1990); and Ausubel et al., eds., Current Protocols in Molecular Biology (1994).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage & Caruthers, Tetrahedron Lett. 22: 1859-1862 (1981), using an automated synthesizer, as described in Van Devanter et. al., Nucleic Acids Res. 12: 6159-6168 (1984). Purification of oligonucleotides is performed using any art-recognized strategy, e.g., native acrylamide gel electrophoresis or anion-exchange HPLC as described in Pearson & Reanier,

### J. Chrom. 255: 137-149 (1983).

The sequence of the cloned wild-type enzyme genes, synthetic oligonucleotides, and polynucleotides can be verified after cloning using, e.g., the chain termination method for sequencing double-stranded templates of Wallace et al., Gene 16: 21-26 (1981).

### Purification of Peptide- and Other-Conjugates

The products produced by the for use in the methods of the invention can be used without purification. However, it is usually preferred to recover the product. Standard, well-known techniques for recovery of peptides such as thin or thick layer chromatography, column chromatography, ion exchange chromatography, or membrane filtration can be used. It is preferred to use membrane filtration, more preferably utilizing a reverse osmotic membrane, or one or more column chromatographic techniques for the recovery as is discussed hereinafter and in the literature cited herein. For instance, membrane filtration wherein the membranes have molecular weight cutoff of about 3000 to about 10,000 can be used to remove proteins such as prenyltransferases. Nanofiltration or reverse osmosis can then be used to remove salts and/or purify the product. Nanofilter membranes are a class of reverse osmosis membranes that pass monovalent salts but retain polyvalent salts and uncharged solutes larger than about 100 to about 2,000 Daltons, depending upon the membrane used.

### VI. Pharmaceutical Compositions

In another aspect, the invention provides a pharmaceutical composition. The pharmaceutical composition includes a pharmaceutically acceptable diluent and a covalent conjugate between a substrate (peptide, glycolipid, aglycone, etc.) and a peptide-conjugate of the invention.

An exemplary conjugate is formed between a non-naturally-occurring, water-soluble polymer, therapeutic moiety or biomolecule and a glycosylated or non-glycosylated peptide. The polymer, therapeutic moiety or biomolecule is conjugated to the peptide via a lipid linking group interposed between and covalently linked to both the peptide and the polymer, therapeutic moiety or biomolecule.

Pharmaceutical compositions of the invention are suitable for use in a variety of drug delivery systems. Suitable formulations for use in the present invention are found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions may be formulated for any appropriate manner of administration, including for example, topical, oral, nasal, intravenous, intracranial, intraperitoneal, subcutaneous or intramuscular administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable matrices, such as microspheres (e.g., polylactate polyglycolate), may also be employed as carriers for the pharmaceutical compositions of this invention. Suitable biodegradable microspheres are disclosed, for example, in U.S. Pat. Nos. 4,897,268 and 5,075,109.

Commonly, the pharmaceutical compositions are administered subcutaneously or parenterally, e.g., intravenously. Thus, the invention provides compositions for parenteral administration which comprise the compound dissolved or suspended in an acceptable carrier, preferably an aqueous carrier, e.g., water, buffered water, saline, PBS and the like. The compositions may also contain detergents such as Tween 20 and Tween 80; stabilizers such as mannitol, sorbitol, sucrose, and trehalose; and preservatives such as EDTA and m-cresol. The compositions may contain pharmaceutically acceptable auxiliary substances as required to approximate physiological conditions, such as pH adjusting and buffering agents, tonicity adjusting agents, wetting agents, detergents and the like.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably from 5 to 9 and most preferably from 7 and 8.

In some embodiments the peptide-conjugates of the invention can be incorporated into liposomes formed from standard vesicle-forming lipids. A variety of methods are available for preparing liposomes, as described in, e.g., Szoka et al., Ann. Rev. Biophys. Bioeng. 9: 467 (1980), U.S. Pat. Nos. 4,235,871, 4,501,728 and 4,837,028. The targeting of liposomes using a variety of targeting agents (e.g., the sialyl galactosides of the invention) is well known in the art (see, e.g., U.S. Pat. Nos. 4,957,773 and 4,603,044).

Standard methods for coupling targeting agents to liposomes can be used. These methods generally involve incorporation into liposomes of lipid components, such as phosphatidylethanolamine, which can be activated for attachment of targeting agents, or derivatized lipophilic compounds, such as lipid-derivatized glycopeptides of the invention.

Targeting mechanisms generally require that the targeting agents be positioned on the surface of the liposome in such a manner that the target moieties are available for interaction with the target, for example, a cell surface receptor. The carbohydrates of the invention may be attached to a lipid molecule before the liposome is formed using methods known to those of skill in the art (e.g., alkylation or acylation of a hydroxyl group present on the carbohydrate with a long chain alkyl halide or with a fatty acid, respectively). Alternatively, the liposome may be fashioned in such a way that a connector portion is first incorporated into the membrane at the time of forming the membrane. The connector portion must have a lipophilic portion, which is firmly embedded and anchored in the membrane. It must also have a reactive portion, which is chemically available on the aqueous surface of the liposome. The reactive portion is selected so that it will be chemically suitable to form a stable chemical bond with the targeting agent or carbohydrate, which is added later. In some cases it is possible to attach the target agent to the connector molecule directly, but in most instances it is more suitable to use a third molecule to act as a chemical bridge, thus linking the connector molecule which is in the membrane with the target agent or carbohydrate which is extended, three dimensionally, off of the vesicle surface.

The compounds prepared by the methods of the invention may also find use as diagnostic reagents. For example, labeled compounds can be used to locate areas of inflammation or tumor metastasis in a patient suspected of having an inflammation. For this use, the compounds can be labeled with ¹²⁵I, ¹⁴C, or tritium.

Preparative methods for species of use in preparing the compositions of the invention are generally set forth in various patent publications, e.g., US 20040137557; WO 04/083258; and WO 04/033651. The following examples are provided to illustrate the conjugates, and methods and of the present invention, but not to limit the claimed invention.

### EXAMPLES

### Example 1

### Production of PEG-Myristoylated GCSF

Production of GCSF has been described previously (see U.S. Pat. Pub. No. 20040077836 and U.S. patent application Ser. No. 11/166,404 (filed Jun. 23, 2005)). To facilitate the addition of the myristoyl group, a mutant GCSF can be synthesized which includes an N-terminal amino acid sequence that satisfies the N-terminal consensus sequence requirements described in Maurer-Stroh et al., J. Mol. Biol., 317:523-540 (2002). Production of mutant peptides are routine and well-known in the art and are further described in Sambrook and Russell, Molecular Cloning, A Laboratory Manual (3rd ed. 2001).

Preparation of G-CSF-Myristoyl-20 kDa-PEG

G-CSF produced in E. coli will be dissolved at 2.5 mg/mL in 50 mM Tris-HCl, 0.15 M NaCl, 0.05% NaN₃, pH 7.2. The solution will be incubated with 1 mM 20 kDaPEG-myristoyl-CoA and 0.1 U/mL of Nmt at 32° C. for 2 days. After 2 days, the reaction mixture will be purified using a Toso Haas G3000SW preparative column using PBS buffer (pH 7.1). The product of the reaction can be analyzed using SDS-PAGE according to the procedures and reagents supplied by Invitrogen. Samples of native and lipoPEGylated G-CSF can also be analyzed by MALDI-TOF MS.

Additional Preparation of G-CSF-Myristoyl-40 kDa-PEG

G-CSF (960 mcg) in 3.2 mL of packaged buffer can be concentrated by ultrafiltration using an UF filter (MWCO 5K) and then reconstituted with 1 mL of 25 mM MES buffer (pH 6.2, 0.005% NaN₃). 40 kDa PEG-Myristoyl-CoA (6 mg, 9.24 mM) and Nmt (40 μL, 0.04 U) can be then added and the resulting solution incubated at room temperature.

### Example 2

### Production of PEG-Palmitoylated GCSF

Production of GCSF has been described previously (see U.S. Pat. Pub. No. 20040077836 and U.S. patent application Ser. No. 11/166,404 (filed Jun. 23, 2005)). To facilitate the addition of the palmitoyl group, a mutant GCSF can be synthesized which includes a consensus sequence for the palmitoyltransferase as described in Smotrys et al., Annu. Rev. Biochem., 73:559-587 (2004). Production of mutant peptides are routine and well-known in the art and are further described in Sambrook and Russell, Molecular Cloning, A Laboratory Manual (3rd ed. 2001).

Preparation of G-CSF-Palmitoyl-20 kDa-PEG

G-CSF produced in E. coli will be dissolved at 2.5 mg/mL in 50 mM Tris-HCl, 0.15 M NaCl, 0.05% NaN₃, pH 7.2. The solution will be incubated with 1 mM 20 kDa-palmitoyl-CoA and 0.1 U/mL of S-palmitoyltransferase at 32° C. for 2 days. After 2 days, the reaction mixture will be purified using a Toso Haas G3000SW preparative column using PBS buffer (pH 7.1). The product of the reaction can be analyzed using SDS-PAGE according to the procedures and reagents supplied by Invitrogen. Samples of native and lipoPEGylated G-CSF can also be analyzed by MALDI-TOF MS.

Additional Preparation of G-CSF-Palmitoyl-30 kDa-PEG

G-CSF (960 mcg) in 3.2 mL of packaged buffer can be concentrated by utrafiltration using an UF filter (MWCO 5K) and then reconstituted with 1 mL of 25 mM MES buffer (pH 6.2, 0.005% NaN₃). 30 kDa PEG-Palmitoyl-CoA (6 mg, 9.24 mM) and S-palmitoyltransferase (40 μL, 0.04 U) can be then added and the resulting solution incubated at room temperature.

### Example 3

### Production of PEG-Farnesylated IFN-α

Preparation of Interferon-α-2b-Farnesyl-PEG-20 KDa

Production of IFN-α 2b has been described previously (see PCT App. No. PCT/US05/______ (filed Sep. 12, 2005, Attorney Docket No. 040853-01-5161)). To facilitate the addition of the farnesyl group, a mutant IFN-α can be synthesized which includes a consensus sequence for the farnesyltransferase as described in Bukhtiyarov et al., J. Bio. Chem., 270(32):19035-19040 (1995). Production of mutant peptides are routine and well-known in the art and are further described in Sambrook and Russell, Molecular Cloning, A Laboratory Manual (3rd ed. 2001).

IFN-α-2b (2 mL, 4.0 mg, 0.2 micromoles) can be buffer exchanged twice with 10 mL of washing buffer and then concentrated to a volume of 0.3 mL using a Centricon centrifugal filter, 5 KDa MWCO. The IFN-α-2b can be reconstituted from the spin cartridge using 2.88 mL of Reaction Buffer and then 20 kDa PEG-farnesyl diphosphate (12 micromoles, 0.15 mL of an 80 mM solution in Reaction Buffer) and farnesyltransferase (0.06 mL, 58 mU), can be added to the reaction mixture. The reaction can be incubated at 32° C. for 40 hours under a slow rotary movement and monitored by SDS PAGE. The product, interferon-alpha-2b-farnesyl-PEG-20 KDa, can be analyzed by MALDI and SDS-PAGE.

Preparation of Interferon-Alpha-2b-Farnesyl-PEG-30 KDa

The IFN-alpha-2b (2 mL, 4.0 mg, 0.2 micromoles) can be buffer exchanged twice with 10 mL of washing buffer and then concentrated to 0.3 mL using a Centricon centrifugal filter, 5 KDa MWCO. The IFN-alpha-2b can be reconstituted from the spin cartridge using 2.98 mL of reaction buffer and then 30 kDa PEG-farnesyl diphosphate (26.3 mg, 0.875 micromoles in 0.75 mL of Reaction Buffer), and farnesyltransferase (0.06 mL, 258 mU) can be added to the reaction mixture to bring the total volume to 4.0 mL. The reaction can be incubated at 32° C. for 40 hours under a slow rotary movement and the reaction monitored by SDS PAGE at 0 h and 40 h.

Preparation of Interferon-alpha-2b-farnesyl-PEG-60 KDa

The IFN-alpha-2b (3.2 mg, 0.17 micromoles) can be reconstituted with 0.64 mL of Reaction Buffer and 60 kDa PEG-farnesyl diphosphate (32 mg, 0.53 micromoles dissolved in 1.6 mL of Reaction Buffer, 0.17 mM final reaction concentration), and farnesyltransferase (0.24 mL, 220 mU) can be added to the reaction mixture to bring the total volume to 3.2 mL. The reaction mixture can be incubated at 32° C. for 40 hours under a slow rotary movement and was monitored by SDS PAGE gel electrophoresis at time points of 0 h and 40 h.

### Example 4

### Production of LipoPEGylated EPO

The following example details methods of modifying an EPO peptide that is expressed in Chinese Hamster Ovary cells (CHO cells). Production of EPO has been described previously, see U.S. patent application Ser. No. 11/144,223. If necessary to facilitate the addition of a PEG-farnesyl-sialyl group, a mutant EPO can be synthesized which includes a consensus sequence for the sialic acid-specific 9(7)-O-acetyltransferase as described in Satake and Varki, J. Bio. Chem., 278(10):7942-7948 (2003). Production of mutant peptides are routine and well-known in the art and are further described in Sambrook and Russell, Molecular Cloning, A Laboratory Manual (3rd ed. 2001).

### Purification of EPO on Superdex75

A Superdex 75 column can be equilibrated in 100 mM MES buffer pH 6.5 containing 150 mM NaCl at a flow rate of 5 mL/min. The EPO product can be loaded on to the column and eluted with the equilibration buffer. The eluate can be monitored for absorbance at 280 nm and conductivity. SDS-PAGE can be used to determine which pooled peak fractions contains the EPO and can then be used in further experiments.

### Preparation of EPO-SA-Farnesyl-PEG-60 KDa

The reaction can be carried out by incubating 1 mg/mL EPO in 100 mM Tris HCl pH 7.5 or MES pH 6.5 containing 150 mM NaCl, 0.5 mM CMP-N-acetyl-neuraminic acid-farnesyl-60 kDa PEG, 0.02% sodium azide, and 200 mU/mL of purified sialic acid-specific 9(7)-O-acetyltransferase at 32° C. for 16 hours.

### Preparation of EPO-SA-Farnesyl-PEG-60 KDa

The reaction can be carried out by incubating 1 mg/mL EPO in 100 mM Tris HCl pH 7.5 or MES pH 6.5 containing 150 mM NaCl, 0.5 mM CMP-N-acetyl-neuraminic acid-farnesyl-60 kDa PEG, 0.02% sodium azide, and 200 mU/mL of Bacillus lipase at 32° C. for 16 hours.

### Example 5

### Production of LipoPEGylated hGH

The following Example illustrates the preparation of a myristoylated hGH protein. Production of hGH has been described previously, see U.S. patent application Ser. No. 11/033,365. To facilitate the addition of the myristoyl group, a mutant GCSF can be synthesized which includes an N-terminal amino acid sequence that satisfies the N-terminal consensus sequence requirements described in Maurer-Stroh et al., J. Mol. Biol., 317:523-540 (2002). Production of mutant peptides are routine and well-known in the art and are further described in Sambrook and Russell, Molecular Cloning, A Laboratory Manual (3rd ed. 2001).

hGH (4.0 mL, 6.0 mg, 0.27 micromoles) can be buffer exchanged twice with 15 mL of Washing Buffer (20 mM HEPES, 150 mM NaCl, 0.02% NaN₃, pH 7.4) and once with Reaction Buffer (20 mM HEPES, 150 mM NaCl, 5 mM MnCl₂, 5 mM MgCl₂, 0.02% NaN₃, pH 7.4) then concentrated to 2.0 mL using a Centricon centrifugal filter, 5 KDa MWCO.

hGH can then be combined with 30 KDa-PEG-myristoyl-CoA (16 mg, 0.533 micromoles) and Nmt (0.375 mL, 375 mU). The reaction can be incubated at 32° C. with gentle shaking for 22 h. The reaction can be monitored by SDS PAGE at 0 h and 22 h. The extent of reaction can be determined by SDS-PAGE gel.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A peptide conjugate comprising:
i) a peptide; and
ii) a modified lipid,
wherein
said modified lipid comprises at least one lipid linking group and at least one modifying group; and
said modifying group is covalently attached to said peptide at a preselected glycosyl and/or amino acid residue of said peptide via the lipid linking group.

2. The peptide conjugate of claim 1, wherein the modified lipid is a member selected from: 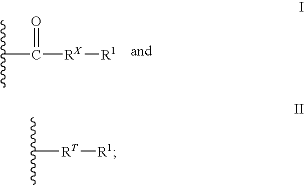
wherein
n is an integer selected from 1 to 20;
R¹ is the modifying group; and
R^{X} is a member selected from substituted or unsubstituted, saturated or unsaturated C₁-C₄₀ alkyl;
R^{T} comprises at least one moiety which has a structure according to the formula: 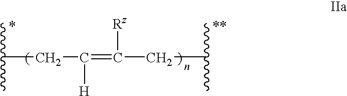
wherein
R^{z} is a member selected from H and substituted or unsubstituted methyl; [symbol]
and [symbol] describe the points of attachment between said moiety and the remainder of the main chain of the modified lipid; [symbol]
describes the point of attachment between the modified lipid and either the glycosyl residue or the amino acid residue of the peptide.

3. The peptide conjugate of claim 2, wherein the modified lipid is Formula II, and Formula II has a structure according to: 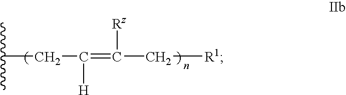
wherein
n is an integer from 1 to 6.

4. The peptide conjugate of claim 3, wherein at least one of said modified lipids is conjugated to said peptide through a sulfur atom on one or more cysteine residues near the C-terminal end of the protein.

5. The peptide conjugate of claim 4, wherein at least one of said modified lipids is conjugated to a single C-terminal cysteine residue that is embedded within a C-terminal amino acid sequence of CAAX,
wherein
A is any aliphatic amino acid, and
X is a member selected from methionine, glutamine, serine and leucine.

6. The peptide conjugate of claim 5, wherein n is 3.

7. The peptide conjugate of claim 6, wherein X is a member selected from methionine, glutamine and serine.

8. The peptide conjugate of claim 5, wherein n is 4.

9. The peptide conjugate of claim 6, wherein X is leucine.

10. The peptide conjugate of claim 4, wherein at least two modified lipids are conjugated separately to two cysteine residues, and said cysteine residues are embedded within a C-terminal amino acid sequence which is a member selected from: cysteine-cysteine and cysteine-X²-cysteine,
wherein
X² is any amino acid.

11. The peptide conjugate of claim 2, wherein said modified lipid is 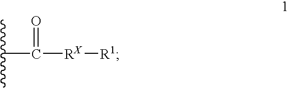
wherein
R^{X} is a member selected from substituted or unsubstituted, saturated or unsaturated C₁-C₄₀ alkyl.

12. The peptide conjugate of claim 11, wherein said R^{X} is a member selected from C₁₄ to C₁₈ alkyl.

13. The peptide conjugate of claim 11, wherein said R^{X} is C₁₄.

14. The peptide conjugate of claim 13, wherein at least one of said modified lipids is conjugated to the peptide through a thioester linkage with one or more cysteine residues of the peptide.

15. The peptide conjugate of claim 14, wherein said peptide comprises a first cysteine residue, and said first cysteine residue is near one of the peptide termini.

16. The peptide conjugate of claim 15, wherein said first cysteine residue is near the C-terminus of the peptide, and said peptide further comprises a second cysteine residue, and said second cysteine residue is conjugated through a sulfur atom on one or more cysteine residues to a modified lipid having a structure according to the following formula: 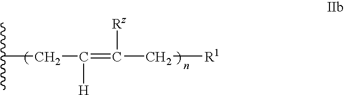
wherein
said first cysteine residue is internal relative to said second cysteine residue.

17. The peptide conjugate of claim 15, wherein said first cysteine residue is near the N-terminus of the peptide, and the peptide is further conjugated through a nitrogen atom at its N-terminus to a modified lipid having a structure according to the following formula: 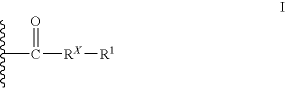
wherein
said first cysteine residue near the N-terminus of the peptide is internal relative to the N-terminus.

18. The peptide conjugate of claim 16, wherein said modified lipid is attached to said peptide through a nitrogen atom.

19. The peptide conjugate of claim 18, wherein R^{X} is a member selected from C₁₄ to C₁₈ alkyl.

20. The peptide conjugate of claim 19, wherein said modified lipid is conjugated to the peptide through a nitrogen atom on an N-terminal glycine residue.

21. The peptide conjugate of claim 20, wherein the N-terminal glycine residue is located at position in the peptide sequence which is a member selected from one and two.

22. The peptide conjugate of claim 1, wherein said modifying group is a water soluble polymer which is a member selected from poly(ether), poly(saccharide) and poly(peptide).

23. The peptide conjugate of claim 22, wherein said poly(peptide) is an enzyme.

24. The peptide conjugate of claim 22, wherein said poly(saccharide) is poly(sialic acid).

25. The peptide conjugate of claim 22, wherein said poly(ether) is a poly(ethylene glycol) which is a member selected from linear PEG and branched PEG.

26. The peptide conjugate of claim 2, wherein said modified lipid has a structure which is a member selected from the following formulas: 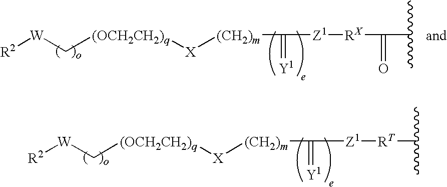
in which
R² is a member selected from H, substituted or unsubstituted alkyl, substituted or unsubstituted aryl, substituted or unsubstituted heteroaryl, substituted or unsubstituted heterocycloalkyl, substituted or unsubstituted heteroalkyl, e.g., acetal, OHC—, H₂N—CH₂CH₂—, HS—CH₂CH₂—, and —(CH₂)_{q}C(Y¹)Z²; -sugar-nucleotide, or protein;
R^{X} is a member selected from substituted or unsubstituted, saturated or unsaturated C₁-C₄₀ alkyl;
q is an integer selected from 1 to 2500;
e is an integer selected from 0 and 1;
m and o are integers independently selected from 0 to 20;
Z¹ is a member selected from O, S, N—R⁴, —(CH₂)ₚC(Y²)V, —(CH₂)ₚU(CH₂)ₙC(Y²)ᵥ;
X, Y¹, Y², W and U are independently selected from O, S, N—R⁴;
V is a member selected from OH, NH₂, halogen, S—R⁵, the alcohol component of activated esters, the amine component of activated amides, sugar-nucleotides, and proteins;
p, s and v are integers independently selected from 0 to 20; and
R³, R⁴ and R⁵ are independently selected from H, substituted or unsubstituted alkyl, substituted or unsubstituted heteroalkyl, substituted or unsubstituted aryl, substituted or unsubstituted heterocycloalkyl and substituted or unsubstituted heteroaryl.

27. The peptide conjugate of claim 2, wherein said PEG moiety is branched PEG and said modified lipid has a structure which is a member selected from the following formulas: 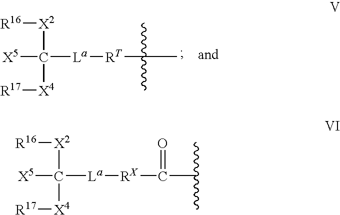
wherein
L^{a} is a linker selected from a bond, substituted or unsubstituted alkyl and substituted or unsubstituted heteroalkyl;
n is an integer selected from 1 to 20;
R^{X} is a member selected from substituted or unsubstituted, saturated or unsaturated C₁-C₄₀ alkyl;
R^{z} is a member selected from H and substituted or unsubstituted methyl;
R¹⁶ and R¹⁷ are independently selected polymeric arms;
X² and X⁴ are independently selected linkage fragments joining polymeric moieties R¹⁶ and R¹⁷ to C; and
X⁵ is a non-reactive group.

28. The peptide conjugate of claim 3, wherein said modified lipid has a structure according to the following formula: 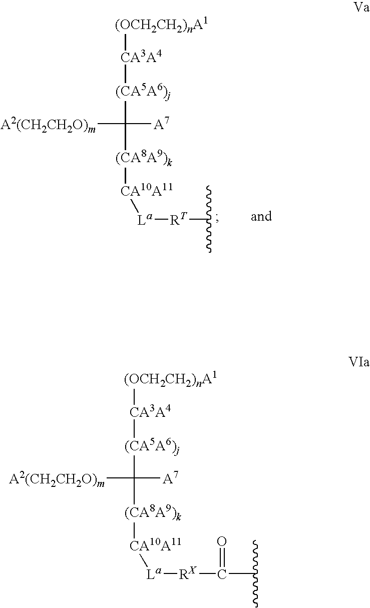
wherein
A¹, A², A³, A⁴, A⁵, A⁶, A⁷, A⁸, A⁹, A¹⁰ and A¹¹ are members independently selected from H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —NA¹²A¹³, —OA¹² and —SiA¹²A¹³
wherein
A¹² and A¹³ are members independently selected from substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

29. The peptide conjugate of claim 1, wherein said modifying group is a water insoluble polymer which is a member selected from poly(vinyl alcohols), polyamides, polyalkylenes, polyacrylamides, polyalkylene glycols and polyalkylene oxides.

30. A method of preparing the peptide conjugate of claim 1, said method comprising:
(a) contacting a peptide with
(i) a modified lipid precursor having a formula selected from 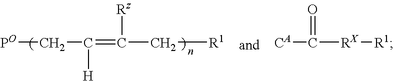
wherein
P^{O} is a member selected from monophosphate and diphosphate;
C^{A} is a member selected from N-hydroxysuccinimidyl, N-hydroxybenztriazolyl, halogen, substituted or unsubstituted imidazolyl, thioethers, p-nitrophenyl ethers, alkyl, alkenyl, alkynyl and aromatic ethers, Coenzyme A and derivatives thereof;
R^{X} is a member selected from substituted or unsubstituted, saturated or unsaturated C₁-C₄₀ alkyl;
R^{z} is a member selected from H and substituted or unsubstituted methyl;
R¹ is a modifying group which is a member selected from a water soluble polymer, a water insoluble polymer, a therapeutic moiety, and a diagnostic moiety; and
(ii) an enzyme for which said modified lipid precursor is a substrate, under conditions appropriate to link said modified lipid precursor to said peptide, thereby preparing said conjugate.

31. A pharmaceutical formulation comprising an effective amount of a peptide conjugate according to claim 1, and a pharmaceutically acceptable excipient.

32. The peptide conjugate of claim 2, wherein said modified lipid is Formula II, and wherein said Formula II is a member selected from: [chemical structure]
